(19) United States
(12) Patent Application Publication
(10) Pub. No.: US 2026/0297031 A1
(43) Pub. Date: Oct. 1, 2026

(54) HYDROXYLAMINE-BASED BCR-ABL1 INHIBITORS FOR TREATMENT OF CANCER

(71) Applicant: University of Georgia Research Foundation, Inc., Athens, GA (US)

(72) Inventors: David CRICH, Athens, GA (US); Jarvis Dawson HILL, Athens, GA (US)

(21) Appl. No.: 19/476,206

(22) PCT Filed: Apr. 3, 2024
(86) PCT No.: PCT/US2024/022712

§ 371 Date: Oct. 17, 2025

Related U.S. Application Data
(60) Provisional application No. 63/497,469, filed on Apr. 21, 2023.

(51) Int. Cl. C07D 215/54; A61K 31/496; C07D 401/04

(52) U.S. Cl. CPC C07D 215/54; A61K 31/496; C07D 401/04

(57) ABSTRACT

In accordance with the purpose(s) of the present disclosure, as embodied and broadly described herein, the disclosure, in one aspect, relates to scaffold molecules that inhibit breakpoint cluster region, chromosome 22 (BCR) and Abelson proto-oncogene 1, chromosome 9 (ABL1) in a subject, methods of making same, pharmaceutical compositions comprising same, and methods of treating cancers involving aberrant BCR and ABL1 activity. In another aspect, the compounds described herein can evade P-gp-mediated efflux in a subject, which makes the compounds effective in treating a Philadelphia chromosome positive (Ph+) leukemia.

### CROSS-REFERENCE TO RELATED APPLICATIONS

This application claims the benefit of and priority to co-pending U.S. Provisional Patent Application No. 63/497,469, filed on Apr. 21, 2023, the contents of which are incorporated by reference herein in their entireties.

### STATEMENT REGARDING FEDERALLY SPONSORED RESEARCH OR DEVELOPMENT

This invention was made with government support under R21 GM144753 awarded by the NIH. The government has certain rights in the invention. (37 CFR 401.14 f (4)).

### BACKGROUND

Chronic myeloid leukemia (CML) comprises 15-20% of all adult leukemia cases worldwide, and is characterized by a reciprocal translocation (t(9;22)(q34;q11) of DNA between chromosomes 9 and 22 in hematologic progenitor cells, that results in fusion of the ABL1 (Abelson proto-oncogene 1, chromosome 9) and BCR (breakpoint cluster region, chromosome 22) genes on the Philadelphia chromosome (Ph).¹⁻⁵ Upon approval in 2001, imatinib (Gleevec®) (1), a BCR-ABL1 targeted tyrosine kinase inhibitor (TKI) ushered in a new era of targeted drug discovery and turned a once fatal leukemia into one in which the lifespan of patients now approaches that of the general population (FIG. 1A).⁶⁻¹¹ Unfortunately, acquired clinical resistance to imatinib, which appears in roughly 40% of all CML, has become a significant challenge in CML management.¹²⁻¹⁶

The search for drugs to treat CML in all of its phases with acquired resistance to imatinib has led to the discovery of second generation BCR-ABL1 inhibitors such as dasatinib¹⁷ (2), nilotinib¹⁸ (3) and bosutinib¹⁹ (4) and third-generation inhibitors ponatinib²⁰ (5) and asciminib²¹ (6) (FIG. 1A). Unfortunately, second- and third-generation BCR-ABL1 inhibitors nilotinib (3), and ponatinib (5) have serious potential side-effects due to inhibition of the human Ether-b-go-go-Related Gene (hERG) potassium ion channel which can lead to QT-interval prolongation, significantly limiting their use in the treatment of CML.²²⁻²⁶ Indeed, nilotinib (3) carries a black box warning for QT prolongation and sudden death while ponatinib (5) carries a black box warning for vascular occlusion, heart failure and hepatotoxicity.

While dasatinib (2), bosutinib (4) and asciminib (6) have reduced affinity for the hERG ion channel, these second- and third-generation BCR-ABL1 inhibitors are potent substrates for efflux transporters P-glycoprotein (P-gp) and/or breast cancer resistance protein (BCRP), which confer indirect inhibitor resistance through active drug efflux.²⁷⁻³⁰ Thus, developing selective, potent inhibitors of BCR-ABL1 with minimal affinity for the hERG ion channel and reduced drug efflux is of significant interest for the clinical management of Ph+ CML and represents an unmet medical need.

### SUMMARY

In accordance with the purpose(s) of the present disclosure, as embodied and broadly described herein, the disclosure, in one aspect, relates to scaffold molecules that inhibit breakpoint cluster region, chromosome 22 (BCR) and Abelson proto-oncogene 1, chromosome 9 (ABL1) in a subject, methods of making same, pharmaceutical compositions comprising same, and methods of treating cancers involving aberrant BCR and ABL1 activity. In another aspect, the compounds described herein can reduce or prevent P-gp-mediated efflux in a subject, which makes the compounds effective in treating a Philadelphia chromosome positive (Ph+) leukemia.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1C show (A) the chemical structures of approved BCR-ABL1 inhibitors and their reported in vitro liabilities; (B) a proposed hydroxylamine analog (hydroxalog) (7) of bosutinib (4); (C) Chemical synthesis of 7; and (D) the chemical structures of exemplary hydroxylamine-based BCR-ABL1 inhibitors in this study: 7, 15.

FIG. 2 shows compound 7 displays potent antiproliferative activity in patient-derived Ph+ CML cell lines MEG-01 and KU812 (p210 BCR-ABL1+), reduced activity in SUP-B15 (p190 BCR-ABL1+) and minimal activity in Molt-4 (BCR-ABL1−) (all 72 h dosing period). For all antiproliferative assays, points indicate the mean, and error bars indicate SD; n=3 biological replicates.

FIGS. 3A-3B show the chemical structures of inhibitors and early absorption, distribution, metabolism, excretion and toxicity (ADMET) profile of the inhibitors. (A) Chemical structures of the hydroxylamine-bearing BCR-ABL1 inhibitors used in this study. (B) In vitro absorption, distribution, metabolism, excretion and toxicity (ADMET) parameters of the inhibitors. Values represent the mean of n=2 independent replicates unless otherwise stated. ADMET values for bosutinib and 7 were previously reported.³¹ HEPClᵢₙₜ, intrinsic clearance in hepatocytes; t_{1/2}, half-life; MDCKII, Madine-Darby canine-kidney; MDR1, multidrug resistance 1 (or P-glycoprotein); Pₐₚₚ, apparent permeability; hERG, human Ether-n-go-go-Related Gene. Abbreviations: H, human.

FIGS. 4A-4B show kinase activity and in vitro antiproliferative activity of 19 and 21. (A) Kinase activity (IC₅₀) against mutant BCR-ABL1 and cSRC. Values represent the mean of n=2 independent replicates. Full kinase activity on inhibitors (bosutinib, 7, 15, 17-21) is provided in the Experimental Section. Values for bosutinib and 7 were previously reported.³¹ (B) 21 displays potent antiproliferative activity in the patient-derived Philadelphia chromosome positive (Ph+) leukemia cell line K562 and exhibits only moderate resistance in doxorubicin pretreated K562 cells (K562/Dox) which overexpress P-glycoprotein. For all antiproliferative assays, points indicate mean, and error bards indicate SD; n=3 independent replicates; IC₅₀ values (nM) are reported beside the dose-response curve and represent the mean (95% confidence interval). IC₅₀ values are unadjusted for FBS.

FIG. 5 shows the extended in vitro absorption, distribution, metabolism, excretion and toxicity (ADMET) profile of 21. All values represent the mean of n=2 independent replicates unless otherwise stated. Low drug-drug interactions (DDI) are predicted for 21 with only moderate inhibition of CYP3A4 observed, which was negative in a follow-up time-dependent inhibition (TDI) study in human liver microsomes. Aq. Sol., aqueous solubility; f_{u,plasma}%, percent fraction unbound in plasma; HEPClᵢₙₜ, intrinsic clearance in hepatocytes; t_{1/2}, half-life; CYP (IC₅₀), cytochrome-P450 inhibition. Abbreviations; H, human; M, mouse.

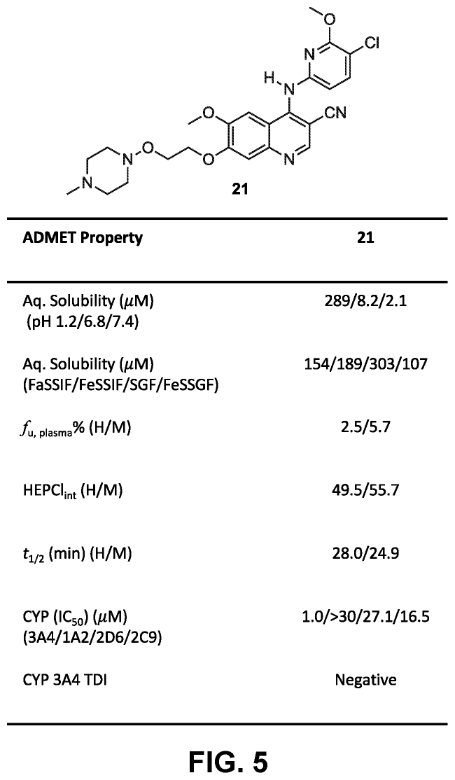

FIG. 6 shows the chemical synthesis of the hydroxylamine-bearing BCR-ABL1 inhibitors and anilines (22, 24). (A) Chemical synthesis of inhibitors (15-21) following a literature procedure³¹(i) Pyridine-hydrochloride (pyr·HCl), aniline, 2-ethoxyethanol, 135° C., 6 h-14 h. (B) Chemical synthesis of aniline (22). (i) H₂ (1 atm), Pd/C, dioxane, r.t., 20 h. (C) Chemical synthesis of aniline (24) by modification of a literature procedure.³² (i) N-Chlorosuccinimide (NCS), DCM, 0° C. to r.t., 2 h. (ii) Benzoyl chloride (BzCl), 4-(dimethylamino)pyridine (DMAP), pyridine, DCM, 0° C. to r.t., 12 h.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an excipient” include, but are not limited to, mixtures or combinations of two or more such excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range. Thus, for example, if a component is in an amount of about 1%, 2%, 3%, 4%, or 5%, where any value can be a lower and upper endpoint of a range, then any range is contemplated between 1% and 5% (e.g., 1% to 3%, 2% to 4%, etc.).

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “IC₅₀,” is intended to refer to the concentration of a substance (e.g., a compound or a drug) that is required for 50% inhibition of a biological process, or component of a process. For example, IC₅₀ refers to the half maximal (50%) inhibitory concentration (IC) of a substance as determined in a suitable assay.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The term “substituted” also includes replacement of a hydrogen atom with deuterium. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The position of a substituent can be defined relative to the positions of other substituents in an aromatic ring. For example, as shown below in relationship to the “R” group, a second substituent can be “ortho,” “para,” or “meta” to the R group, meaning that the second substituent is bonded to a carbon labeled ortho, para, or meta as indicated below. Combinations of ortho, para, and meta substituents relative to a given group or substituent are also envisioned and should be considered to be disclosed.

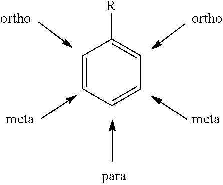

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl. The term “alkyl” also includes saturated hydrocarbon groups where one or more hydrogen atoms are replaced with deuterium (i.e., a deuteriated alkyl group).

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above.

The term “alkenyl” as used herein is a hydrocarbon group of from 2 to 24 carbon atoms with a structural formula containing at least one carbon-carbon double bond. Asymmetric structures such as (A¹A²)C═C(A³A₄) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cyclooctynyl, cyclononynyl, and the like. The term “heterocycloalkynyl” is a type of cycloalkenyl group as defined above and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl. Fused aryl groups including, but not limited to, indene and naphthalene groups are also contemplated.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A'S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{⊚} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{⊚} include=O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R†, —NR†₂, —C(O)R†, —C(O)OR†, —C(O)C(O)R†, —C(O)CH₂C(O)R†, —S(O)₂R†, —S(O)₂NR†₂, —C(S)NR†₂, —C(NH)NR†₂, or —N(Rt)S(O)₂R†; wherein each R† is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R†, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

It is also appreciated that certain compounds described herein can be present as an equilibrium of tautomers. For example, ketones with an α-hydrogen can exist in an equilibrium of the keto form and the enol form.

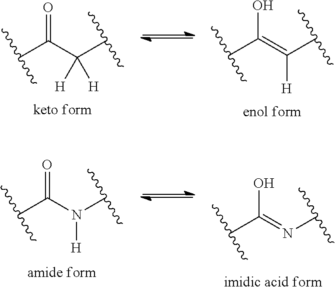 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

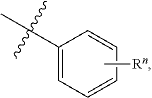

which is understood to be equivalent to a formula:

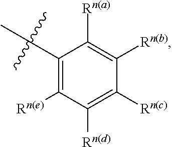

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(b)} is not necessarily halogen in that instance.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a hematological malignancy, breast cancer, and/or another solid malignancy. The effect can be therapeutic in terms of a partial or complete cure of a disease, condition, symptom or adverse effect attributed to the disease, disorder, or condition. The term “treatment” as used herein can include any treatment of a hematological malignancy, breast cancer, and/or another solid tumor in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### Compounds and Methods of Making and Using the Compounds

In one aspect, disclosed herein is a compound having a structure according to structure I or the pharmaceutically acceptable salt thereof

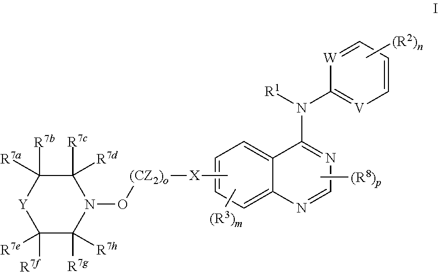

wherein
   R¹ is hydrogen, a substituted or unsubstituted linear or branched alkyl group, a substituted or unsubstituted cycloalkyl group or heterocycloalkyl group, or a substituted or unsubstituted aryl group;
   n is an integer from 1 to 5, where each R² is independently hydrogen, a substituted or unsubstituted linear or branched alkyl group, a substituted or unsubstituted cycloalkyl group or heterocycloalkyl group, a substituted or unsubstituted aryl group, a halide, or an alkoxy group;
   m is an integer from 1 to 3, where each R³ is independently hydrogen, a substituted or unsubstituted linear or branched alkyl group, a substituted or unsubstituted cycloalkyl group or heterocycloalkyl group, a substituted or unsubstituted aryl group, a halide, or an alkoxy group;
   o is an integer from 1 to 10;
   p is 1 or 2;
   V is N or CH;
   W is N or CH;
   X is O or NR⁴, wherein R⁴ is hydrogen, a substituted or unsubstituted linear or branched alkyl group, a substituted or unsubstituted cycloalkyl group or heterocycloalkyl group, or a substituted or unsubstituted aryl group;
   Y is O, NR⁵, or CR^{6a}R^{6b}, wherein R⁵ is hydrogen, deuterium, a substituted or unsubstituted linear or branched alkyl group, a substituted or unsubstituted cycloalkyl group or heterocycloalkyl group, or a substituted or unsubstituted aryl group, and
   R^{6a} and R^{6b} are independently hydrogen, deuterium, a substituted or unsubstituted linear or branched alkyl group, a substituted or unsubstituted cycloalkyl group or heterocycloalkyl group, a substituted or unsubstituted aryl group, or a substituted or unsubstituted amino group;
   R^{7a}, R^{7b}, R^{7c}, R^{7d}, R^{7e}, R^{7f}, R^{7g}, and R^{7h} are independently hydrogen, deuterium, or a substituted or unsubstituted alkyl group;
   R⁸ is cyano; and
   each Z is independently hydrogen or deuterium.

In one aspect, X in structure I is O. In another aspect, Y in structure I is O. In another aspect, Y in structure I is NR⁵, where R⁵ in structure I is a C1 to C5 alkyl group. In another aspect, Y in structure I is CR^{6a}R^{6b}, where R^{6a} is hydrogen and R^{6b} is a substituted or unsubstituted amino group. In another aspect, R¹ in structure I is hydrogen. In another aspect, R³ in structure I is an alkoxy group. In another aspect, R³ in structure I is an alkoxy group and m is 1. In another aspect, o in structure I is an integer from 1 to 5. In another aspect, R² in structure I is a halide and n is 2. In another aspect, R² in structure I is fluoride or chloride at the ortho position. In another aspect, n in structure I is 3 and R² is two halide atoms and one alkoxy group. In another aspect, n in structure I is 3 and R² is two chloride atoms and one methoxy group. In another aspect, p in structure I is 1.

In another aspect, the compound has the structure II or the pharmaceutically acceptable salt thereof

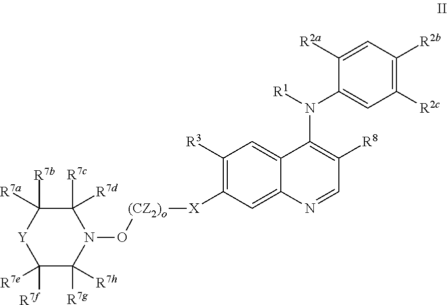

wherein
   R¹ is hydrogen, a substituted or unsubstituted linear or branched alkyl group, a substituted or unsubstituted cycloalkyl group or heterocycloalkyl group, or a substituted or unsubstituted aryl group;
   R^{2a} and R^{2b} are a halide;
   R^{2c} is an alkoxy group;
   R³ is an alkoxy group;
   o is an integer from 1 to 5;
   X is O or NR⁴, wherein R⁴ is hydrogen, a substituted or unsubstituted linear or branched alkyl group, a substituted or unsubstituted cycloalkyl group or heterocycloalkyl group, or a substituted or unsubstituted aryl group;
   Y is O, NR⁵, or CR^{6a}R^{6b}, wherein R⁵ is hydrogen, deuterium, a substituted or unsubstituted linear or branched alkyl group, a substituted or unsubstituted cycloalkyl group or heterocycloalkyl group, or a substituted or unsubstituted aryl group, and
   R^{6a} and R^{6b} are independently hydrogen, deuterium, a substituted or unsubstituted linear or branched alkyl group, a substituted or unsubstituted cycloalkyl group or heterocycloalkyl group, a substituted or unsubstituted aryl group, or a substituted or unsubstituted amino group;
   R^{7a}, R^{7b}, R^{7c}, R^{7d}, R^{7e}, R^{7f}, R^{7g}, and R^{7h} are independently hydrogen, deuterium, or a substituted or unsubstituted alkyl group;
   R^{a} is cyano; and
      each Z is independently hydrogen or deuterium.

In one aspect, X in structure II is O. In another aspect, Y in structure II is O. In another aspect, Y in structure II is NR⁵, where R⁵ is a C1 to C5 alkyl group. In another aspect, Y in structure II is CR^{6a}R^{6b}, where R^{6a} is hydrogen and R^{6b} is a substituted or unsubstituted amino group. In another aspect, R¹ in structure II is hydrogen. In another aspect, R³ in structure II is a C1 to C10 substituted or unsubstituted linear or branched alkoxy group. In another aspect, R³ in structure II is a methoxy group. In another aspect, o is an integer from 1 to 5. In another aspect, R^{2a} and R^{2b} in structure II are chloride. In another aspect, R^{2a} in structure II is chloride and R^{2b} is fluoride. In another aspect, R^{2a} in structure II is fluoride and R^{2b} is chloride. In another aspect, R^{2c} in structure II is a C1 to C10 substituted or unsubstituted linear or branched alkoxy group. In another aspect, R^{2c} in structure II is a methoxy group.

In another aspect, the compound has the structure Ill or the pharmaceutically acceptable salt thereof

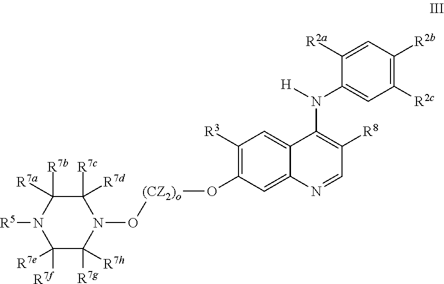

wherein
   R^{2a} and R^{2b} are a halide;
   R^{2c} is an alkoxy group;
   R³ is an alkoxy group;
   o is an integer from 1 to 5;
   R⁵ is hydrogen, deuterium, a substituted or unsubstituted linear or branched alkyl group, a substituted or unsubstituted cycloalkyl group or heterocycloalkyl group, or a substituted or unsubstituted aryl group, and
   R^{7a}, R^{7b}, R^{7c}, R^{7d}, R^{7e}, R^{7f}, R^{7g}, and R^{7h} are independently hydrogen, deuterium, or a substituted or unsubstituted alkyl group;
   R⁸ is cyano; and
      each Z is independently hydrogen or deuterium.

In one aspect, R⁵ in structure Ill is a C1 to C5 alkyl group. In another aspect, R⁵ in structure Ill is a methyl group. In another aspect, R³ in structure Ill is a C1 to C10 substituted or unsubstituted linear or branched alkoxy group. In another aspect, R³ in structure Ill is a methoxy group. In another aspect, o in structure Ill is an integer from 1 to 5 (e.g., 2). In another aspect, R^{2a} in structure Ill is chloride and R^{2b} is fluoride. In another aspect, R^{2a} in structure Ill is fluoride and R^{2b} is chloride. In another aspect, R^{2a} and R^{2b} in structure Ill are chloride. In another aspect, R^{2c} in structure Ill is a C1 to C10 substituted or unsubstituted linear or branched alkoxy group. In another aspect, R^{2c} in structure Ill is a methoxy group. In another aspect, R^{7a}, R^{7b}, R^{7c}, R^{7d}, R^{7e}, R^{7f}, R^{7g}, and R^{7h} in structure Ill are each hydrogen.

In another aspect, the compound has the structure IV or the pharmaceutically acceptable salt thereof

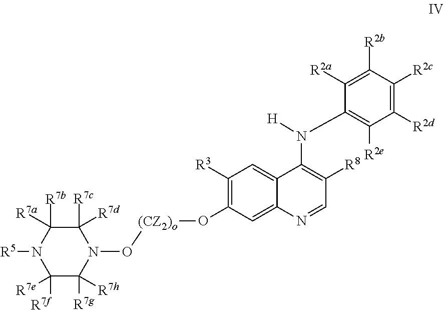

wherein
   R^{2a}-R^{2e} are hydrogen, a halide, or an alkoxy group;
   o is an integer from 1 to 5;
   R³ is hydrogen, deuterium, a substituted or unsubstituted linear or branched alkyl group, a substituted or unsubstituted cycloalkyl group or heterocycloalkyl group, or a substituted or unsubstituted aryl group, and
   R^{7a}, R^{7b}, R^{7c}, R^{7d}, R^{7e}, R^{7f}, R^{7g}, and R^{7h} are independently hydrogen, deuterium, or a substituted or unsubstituted alkyl group;
   R⁸ is cyano; and
   each Z is independently hydrogen or deuterium.

In one aspect, R⁵ in structure IV is a C1 to C5 alkyl group. In another aspect, R⁵ in structure IV is a methyl group. In another aspect, R³ in structure IV is a C1 to C10 substituted or unsubstituted linear or branched alkoxy group. In another aspect, R³ in structure IV is a methoxy group. In another aspect, o in structure IV is an integer from 1 to 5 (e.g., 2). In another aspect, R^{2a} is chloride or fluoride, R^{2c} is fluoride, R^{2d} is C1 to 010 substituted or unsubstituted linear or branched alkoxy group, and R^{2b} and R^{2e} are hydrogen. In another aspect, R^{2a} is chloride or fluoride, R^{2b} is chloride, and R^{2c}, R^{2d}, and R^{2e} are hydrogen. In another aspect, R^{2a} is fluoride or chloride, R^{2b} is C1 to C10 substituted or unsubstituted linear or branched alkoxy group, and R^{2c}, R^{2d}, and R^{2e} are hydrogen. In another aspect, R^{2b} is C1 to C10 substituted or unsubstituted linear or branched alkoxy group, R^{2c} is chloride, and R^{2a}, R^{2d}, and R^{2e} are hydrogen. In another aspect, R^{2a} is C1 to C10 substituted or unsubstituted linear or branched alkoxy group, R^{2c} is chloride, and R^{2b}, R^{2d}, and R^{2e} are hydrogen. In another aspect, the C1 to C10 substituted or unsubstituted linear or branched alkoxy group of R^{2a}-R^{2e} is a methoxy group. In another aspect, R^{7a}, R^{7b}, R^{7c}, R^{7d}, R^{7e}, R^{7f}, R^{7g}, and R^{7h} are each hydrogen.

In another aspect, the compound has the structure V or the pharmaceutically acceptable salt thereof

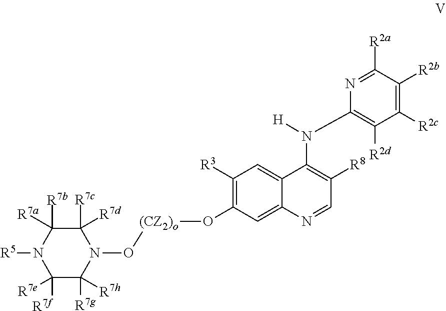

wherein
   R^{2a}-R^{2d} are hydrogen, a halide, or an alkoxy group;
   o is an integer from 1 to 5;
   R³ is hydrogen, deuterium, a substituted or unsubstituted linear or branched alkyl group, a substituted or unsubstituted cycloalkyl group or heterocycloalkyl group, or a substituted or unsubstituted aryl group, and
   R^{7a}, R^{7b}, R^{7c}, R^{7d}, R^{7e}, R^{7f}, R^{7g}, and R^{7h} are independently hydrogen, deuterium, or a substituted or unsubstituted alkyl group;
   R⁸ is cyano; and
   each Z is independently hydrogen or deuterium.

In one aspect, R⁵ in structure V is a C1 to C5 alkyl group. In another aspect, R⁵ in structure V is a methyl group. In another aspect, R³ in structure V is a C1 to C10 substituted or unsubstituted linear or branched alkoxy group. In another aspect, R³ in structure V is a methoxy group. In another aspect, o in structure V is an integer from 1 to 5 (e.g., 2). In another aspect, R^{2a} is C1 to C10 substituted or unsubstituted linear or branched alkoxy group, R^{2b} is a halide, and R^{2c} and R^{2d} are hydrogen. In another aspect, R^{7a}, R^{7b}, R^{7c}, R^{7d}, R^{7e}, R^{7f}, R^{7g}, and R^{7h} are each hydrogen.

In another aspect, the compound has the structure VI or the pharmaceutically acceptable salt thereof

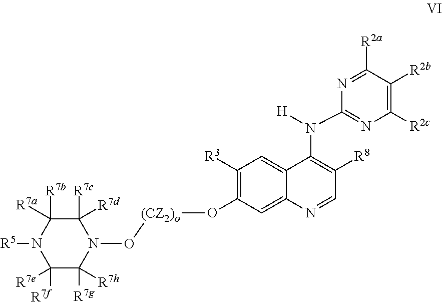

wherein
   R^{2a}-R^{2c} are hydrogen, a halide, or an alkoxy group;
   o is an integer from 1 to 5;
   R³ is hydrogen, deuterium, a substituted or unsubstituted linear or branched alkyl group, a substituted or unsubstituted cycloalkyl group or heterocycloalkyl group, or a substituted or unsubstituted aryl group, and
   R^{7a}, R^{7b}, R^{7c}, R^{7d}, R^{7e}, R^{7f}, R^{7g}, and R^{7h} are independently hydrogen, deuterium, or a substituted or unsubstituted alkyl group;
   R⁸ is cyano; and
   each Z is independently hydrogen or deuterium.

In one aspect, R⁵ in structure VI is a C1 to C5 alkyl group. In another aspect, R⁵ in structure VI is a methyl group. In another aspect, R³ in structure VI is a C1 to C10 substituted or unsubstituted linear or branched alkoxy group. In another aspect, R³ in structure VI is a methoxy group. In another aspect, o in structure VI is an integer from 1 to 5 (e.g., 2). In another aspect, R^{2a} is C1 to C10 substituted or unsubstituted linear or branched alkoxy group, R^{2b} is a halide, and R^{2c} is hydrogen. In another aspect, R^{2a} is methoxy, R^{2b} is chloride, and R^{2c} is hydrogen. In another aspect, R^{7a}, R^{7b}, R^{7c}, R^{7d}, R^{7e}, R^{7f}, R^{7g}, and R^{7h} are each hydrogen.

In another aspect, the compound has the following structure

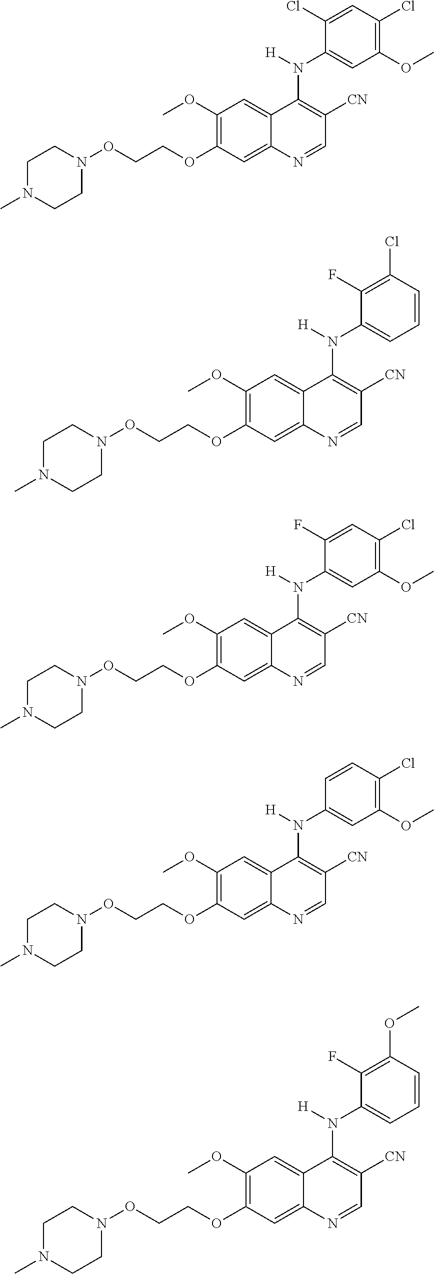

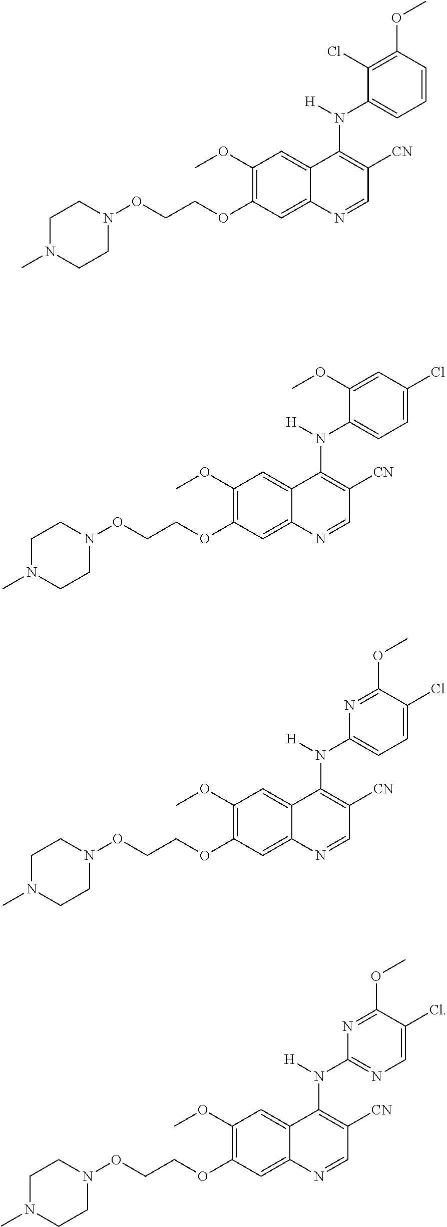 [SMILES: COc1cc2c(Nc3cccc(OC)c3Cl)c(C#N)cnc2cc1OCCON1CCN(C)CC1 ; COc1cc(Cl)ccc1Nc1c(C#N)cnc2cc(OCCON3CCN(C)CC3)c(OC)cc12 ; COc1cc2c(Nc3ccc(Cl)c(OC)n3)c(C#N)cnc2cc1OCCON1CCN(C)CC1 ; COc1cc2c(Nc3ncc(Cl)c(OC)n3)c(C#N)cnc2cc1OCCON1CCN(C)CC1]

### General Synthetic Method

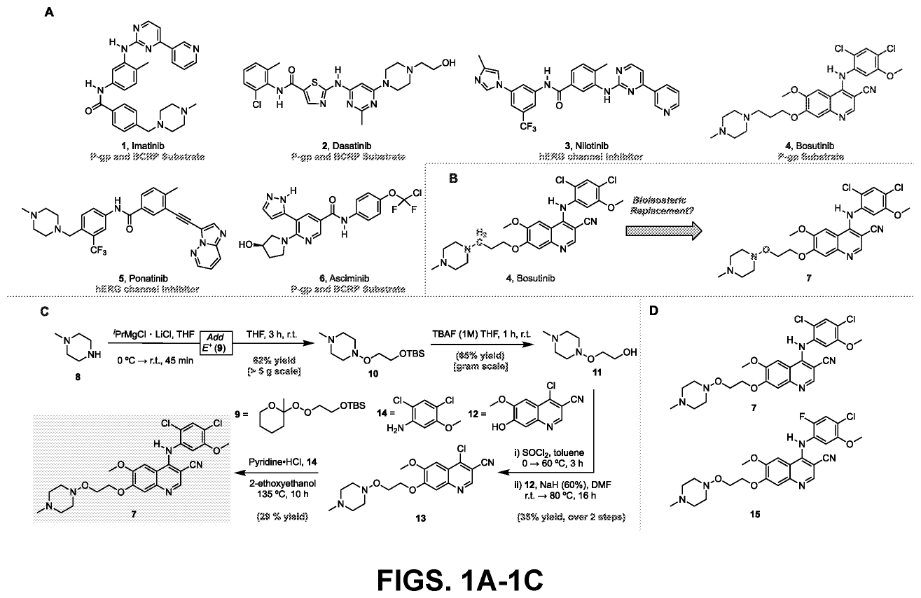

Exemplary methods for producing compounds described herein, as well as characterization information, are provided in the Examples. For example, FIG. 1C provides a non-limiting procedure for producing compounds described herein. Solvents, temperatures, presence or absence of protecting groups, and other reaction conditions may vary according to the specific substituents in the compound being synthesized.

### Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethyl carbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In one aspect, an appropriate dosage level will generally be about 0.01 to 1000 mg of a compound described herein per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

### Methods for Treatment of Cancers in Subjects

In one aspect, disclosed herein is a method for the treatment of a cancer in a subject, the method including the step of administering to the subject a therapeutically effective amount of at least one disclosed compound, or a pharmaceutically acceptable salt thereof, or the disclosed pharmaceutical composition. In some aspects, the subject is a human. In another aspect, the subject has been diagnosed with a need for treatment of the cancer prior to the administering step. In some aspects, the method further includes the step of identifying a subject in need of treatment of the cancer. In one aspect, the cancer is selected from chronic myeloid leukemia, acute lymphoblastic leukemia, glioblastoma, non-small cell lung cancer, colorectal cancer, or prostate cancer.

In another aspect, disclosed herein is a method for inhibiting breakpoint cluster region, chromosome 22 (BCR) and Abelson proto-oncogene 1, chromosome 9 (ABL1) in a subject, including the step of administering to the subject a therapeutically effective amount of at least one disclosed compound, or a pharmaceutically acceptable salt thereof, or a disclosed pharmaceutical composition. In one aspect, the subject is a human.

In another aspect, disclosed herein a for treating a subject having a Philadelphia chromosome positive (Ph+) leukemia, the method comprising administering to the subject an effective amount of at least one disclosed compound, or a pharmaceutically acceptable salt thereof, or a disclosed pharmaceutical composition. One of the most common mechanisms invoked for off-target resistance in chronic myeloid leukemia (CML) is upregulation of the permeability glycoprotein (P-gp; MDR1) efflux pump, a 170 kDa protein encoded by the ABCB1 gene. Unfortunately, most small-molecule targeted therapies for CML are P-gp substrates, including the breakpoint cluster region—Abelson proto-oncogene 1 (BCR-ABL1) first-generation inhibitor imatinib (1) and the second- and third-generation inhibitors dasatinib (2), bosutinib (4), and asciminib (6). The disclosed compounds, or a pharmaceutically acceptable salt thereof, or a disclosed pharmaceutical composition can evade P-gp-mediated efflux in a subject, which makes the compounds described herein suitable for treating Philadelphia chromosome positive (Ph+) leukemias that would benefit from reduced P-gp-mediated efflux, and the consequent minimization of potential off-target resistance by P-gp overexpression.

In one aspect, the compound is administered orally to the subject. In another aspect, the compound is administered at a dosage of from about 50 mg per day to about 1,000 mg per day, or about 50 mg per day, 50 mg per day, 100 mg per day, 150 mg per day, 200 mg per day, 250 mg per day, 300 mg per day, 350 mg per day, 400 mg per day, 450 mg per day, 500 mg per day, 550 mg per day, 600 mg per day, 650 mg per day, 700 mg per day, 750 mg per day, 800 mg per day, 850 mg per day, 900 mg per day, 950 mg per day, or 1,000 mg per day, where any value can be a lower and upper endpoint of a range (e.g., 100 mg per day to 300 mg per day).

As demonstrated in the Examples, the compounds described herein lack mutagenicity and demonstrate comparable metabolic stability to bosutinib. Additionally, the compounds have improved efflux ratios measured in cellular assays. The compounds described herein can be used alone or in combination with other chemotherapeutic agents and/or radiation.

### Aspects

Aspect 1. A compound having structure I or the pharmaceutically acceptable salt thereof

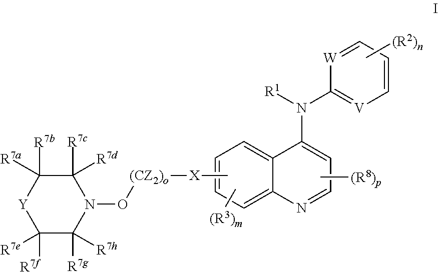

wherein
   R¹ is hydrogen, a substituted or unsubstituted linear or branched alkyl group, a substituted or unsubstituted cycloalkyl group or heterocycloalkyl group, or a substituted or unsubstituted aryl group;
   n is an integer from 1 to 5, where each R² is independently hydrogen, a substituted or unsubstituted linear or branched alkyl group, a substituted or unsubstituted cycloalkyl group or heterocycloalkyl group, a substituted or unsubstituted aryl group, a halide, or an alkoxy group;
m is an integer from 1 to 3, where each R³ is independently hydrogen, a substituted or unsubstituted linear or branched alkyl group, a substituted or unsubstituted cycloalkyl group or heterocycloalkyl group, a substituted or unsubstituted aryl group, a halide, or an alkoxy group;
o is an integer from 1 to 10;
p is 1 or 2;
V is N or CH;
W is N or CH;
X is O or NR⁴, wherein R⁴ is hydrogen, a substituted or unsubstituted linear or branched alkyl group, a substituted or unsubstituted cycloalkyl group or heterocycloalkyl group, or a substituted or unsubstituted aryl group;
Y is O, NR⁵, or CR^{6a}R^{6b}, wherein R⁵ is hydrogen, deuterium, a substituted or unsubstituted linear or branched alkyl group, a substituted or unsubstituted cycloalkyl group or heterocycloalkyl group, or a substituted or unsubstituted aryl group, and
R^{6a} and R^{6b} are independently hydrogen, deuterium, a substituted or unsubstituted linear or branched alkyl group, a substituted or unsubstituted cycloalkyl group or heterocycloalkyl group, a substituted or unsubstituted aryl group, or a substituted or unsubstituted amino group; R^{7a}, R^{7b}, R^{7c}, R^{7d}, R^{7e}, R^{7f}, R^{7g}, and R^{7h} are independently hydrogen, deuterium, or a substituted or unsubstituted alkyl group;
R⁸ is cyano; and
each Z is independently hydrogen or deuterium.

Aspect 2. The compound of Aspect 1, wherein X is O.

Aspect 3. The compound of Aspect 1 or 2, wherein Y is O.

Aspect 4. The compound of Aspect 1 or 2, wherein Y is NR⁵, where R⁵ is a C1 to C5 alkyl group.

Aspect 5. The compound of Aspect 1 or 2, wherein Y is CR^{6a}R^{6b}, where R^{6a} is hydrogen and R^{6b} is a substituted or unsubstituted amino group.

Aspect 6. The compound of any one of Aspects 1-5, wherein R¹ is hydrogen.

Aspect 7. The compound of any one of Aspects 1-6, wherein R³ is an alkoxy group.

Aspect 8. The compound of any one of Aspects 1-6, wherein R³ is an alkoxy group and mis 1.

Aspect 9. The compound of any one of Aspects 1-8, wherein o is an integer from 1 to 5.

Aspect 10. The compound of any one of Aspects 1-9, wherein R² is a halide and n is 2.

Aspect 11. The compound of any one of Aspects 1-10, wherein R² is fluoride or chloride at the ortho position.

Aspect 12. The compound of any one of Aspects 1-10, wherein n is 3 and R² is two halide atoms and one alkoxy group.

Aspect 13. The compound of any one of Aspects 1-10, wherein n is 3 and R² is two chloride atoms and one methoxy group.

Aspect 14. The compound of any one of Aspects 1-10, wherein p is 1.

Aspect 15. The compound of any one of Aspects 1-14, wherein W is CH and V is CH.

Aspect 16. The compound of any one of Aspects 1-14, wherein W is CH and V is N.

Aspect 17. The compound of any one of Aspects 1-14, wherein W is N and V is N

Aspect 18. The compound of Aspect 1, wherein the compound has the structure II or the pharmaceutically acceptable salt thereof

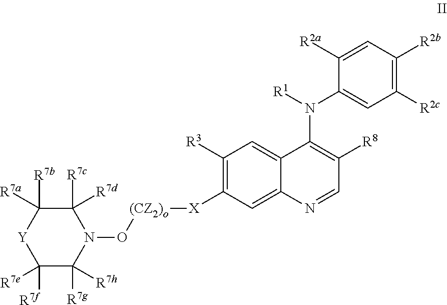

wherein
R¹ is hydrogen, a substituted or unsubstituted linear or branched alkyl group, a substituted or unsubstituted cycloalkyl group or heterocycloalkyl group, or a substituted or unsubstituted aryl group;
R^{2a} and R^{2b} are a halide;
R^{2c} is an alkoxy group;
R³ is an alkoxy group;
o is an integer from 1 to 5;
X is O or NR⁴, wherein R⁴ is hydrogen, a substituted or unsubstituted linear or branched alkyl group, a substituted or unsubstituted cycloalkyl group or heterocycloalkyl group, or a substituted or unsubstituted aryl group;
Y is O, NR⁵, or CR^{6a}R^{6b}, wherein R⁵ is hydrogen, deuterium, a substituted or unsubstituted linear or branched alkyl group, a substituted or unsubstituted cycloalkyl group or heterocycloalkyl group, or a substituted or unsubstituted aryl group, and
R^{6a} and R^{6b} are independently hydrogen, deuterium, a substituted or unsubstituted linear or branched alkyl group, a substituted or unsubstituted cycloalkyl group or heterocycloalkyl group, a substituted or unsubstituted aryl group, or a substituted or unsubstituted amino group;
R^{7a}, R^{7b}, R^{7c}, R^{7d}, R^{7e}, R^{7f}, R^{7g}, and R^{7h} are independently hydrogen, deuterium, or a substituted or unsubstituted alkyl group;
R⁸ is cyano; and
each Z is independently hydrogen or deuterium.

Aspect 19. The compound of Aspect 18, wherein X is O.

Aspect 20. The compound of Aspect 18 or 19, wherein Y is O.

Aspect 21. The compound of Aspect 18 or 19, wherein Y is NR⁵, where R⁵ is a C1 to C5 alkyl group.

Aspect 22. The compound of Aspect 18 or 19, wherein Y is CR^{6a}R^{6b}, where R^{6a} is hydrogen and R^{6b} is a substituted or unsubstituted amino group.

Aspect 123. The compound of any one of Aspects 18 to 22, wherein R¹ is hydrogen.

Aspect 24. The compound of any one of Aspects 18 to 23, wherein R³ is a C1 to C10 substituted or unsubstituted linear or branched alkoxy group.

Aspect 25. The compound of any one of Aspects 18 to 23, wherein R³ is a methoxy group.

Aspect 26. The compound of any one of Aspects 18 to 25, wherein o is an integer from 1 to 5.

Aspect 27. The compound of any one of Aspects 18 to 26, wherein R^{2a} and R^{2b} are chloride.

Aspect 28. The compound of any one of Aspects 18 to 26, wherein R^{2a} is chloride and R^{2b} is fluoride.

Aspect 29. The compound of any one of Aspects 18 to 26, wherein R^{2a} is fluoride and R^{2b} is chloride.

Aspect 30. The compound of any one of Aspects 18 to 29, wherein R^{2c} is a C1 to C10 substituted or unsubstituted linear or branched alkoxy group.

Aspect 31. The compound of any one of Aspects 18 to 29, wherein R^{2c} is a methoxy group.

Aspect 32. The compound of Aspect 1, wherein the compound has the structure Ill or the pharmaceutically acceptable salt thereof

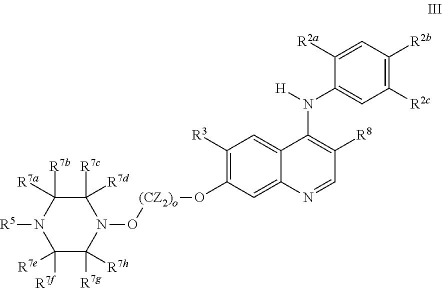

wherein
R^{2a} and R^{2b} are a halide;
R^{2c} is an alkoxy group;
R³ is an alkoxy group;
o is an integer from 1 to 5;
R⁵ is hydrogen, deuterium, a substituted or unsubstituted linear or branched alkyl group, a substituted or unsubstituted cycloalkyl group or heterocycloalkyl group, or a substituted or unsubstituted aryl group, and
R^{7a}, R^{7b}, R^{7c}, R^{7d}, R^{7e}, R^{7f}, R^{7g}, and R^{7h} are independently hydrogen, deuterium, or a substituted or unsubstituted alkyl group;
R⁸ is cyano; and
each Z is independently hydrogen or deuterium.

Aspect 33. The compound of Aspect 32, wherein R⁵ is a C1 to C5 alkyl group.

Aspect 34. The compound of Aspect 32, wherein R⁵ is a methyl group.

Aspect 35. The compound of any one of Aspects 32 to 34, wherein R³ is a C1 to C10 substituted or unsubstituted linear or branched alkoxy group.

Aspect 36. The compound of any one of Aspects 32 to 34, wherein R³ is a methoxy group.

Aspect 37. The compound of any one of Aspects 32-36, wherein o is an integer from 1 to 5.

Aspect 38. The compound of any one of Aspects 32-36, wherein o is 2.

Aspect 39. The compound of any one of Aspects 32 to 38, wherein R^{2a} is chloride and R^{2b} is fluoride.

Aspect 40. The compound of any one of Aspects 32 to 38, wherein R^{2a} is fluoride and R^{2b} is chloride.

Aspect 41. The compound of any one of Aspects 32 to 38, wherein R^{2a} and R^{2b} are chloride.

Aspect 42. The compound of any one of Aspects 32 to 38, wherein R^{2c} is a C1 to C10 substituted or unsubstituted linear or branched alkoxy group.

Aspect 43. The compound of any one of Aspects 32 to 42, wherein R^{2c} is a methoxy group.

Aspect 44. The compound of any one of Aspects 32 to 43, wherein R^{7a}, R^{7b}, R^{7c}, R^{7d}, R^{7e}, R^{7f}, R^{7g}, and R^{7h} are each hydrogen.

Aspect 45. The compound of Aspect 1, wherein the compound has the structure IV or the pharmaceutically acceptable salt thereof

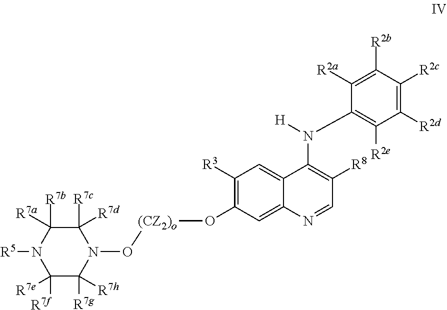

wherein
R^{2a}-R^{2e} are hydrogen, a halide, or an alkoxy group;
o is an integer from 1 to 5;
R³ is hydrogen, deuterium, a substituted or unsubstituted linear or branched alkyl group, a substituted or unsubstituted cycloalkyl group or heterocycloalkyl group, or a substituted or unsubstituted aryl group, and
R^{7a}, R^{7b}, R^{7c}, R^{7d}, R^{7e}, R^{7f}, R^{7g}, and R^{7h} are independently hydrogen, deuterium, or a substituted or unsubstituted alkyl group;
R⁸ is cyano; and
each Z is independently hydrogen or deuterium.

Aspect 46. The compound of Aspect 45, wherein R⁵ is a C1 to C5 alkyl group.

Aspect 47. The compound of Aspect 45, wherein R⁵ is a methyl group.

Aspect 48. The compound of any one of Aspects 45 to 47, wherein R³ is a C1 to C10 substituted or unsubstituted linear or branched alkoxy group.

Aspect 49. The compound of any one of Aspects 45 to 47, wherein R³ is a methoxy group.

Aspect 50. The compound of any one of Aspects 45 to 49, wherein o is an integer from 1 to 5.

Aspect 51. The compound of any one of Aspects 45 to 49, wherein o is 2.

Aspect 52. The compound of any one of Aspects 45 to 51, wherein R^{2a} is chloride or fluoride, R^{2c} is fluoride, R^{2d} is C1 to 010 substituted or unsubstituted linear or branched alkoxy group, and R^{2b} and R^{2e} are hydrogen.

Aspect 53. The compound of any one of Aspects 45 to 51, wherein R^{2a} is chloride or fluoride, R^{2b} is chloride, and R^{2c}, R^{2d}, and R^{2e} are hydrogen.

Aspect 54. The compound of any one of Aspects 45 to 51, wherein R^{2a} is fluoride or chloride, R^{2b} is C1 to C10 substituted or unsubstituted linear or branched alkoxy group, and R^{2a}, R^{2d}, and R^{2e} are hydrogen.

Aspect 55. The compound of any one of Aspects 45 to 51, wherein R^{2b} is C1 to C10 substituted or unsubstituted linear or branched alkoxy group, R^{2c} is chloride, and R^{2a}, R^{2d}, and R^{2e} are hydrogen.

Aspect 56. The compound of any one of Aspects 45 to 51, wherein R^{2a} is C1 to C10 substituted or unsubstituted linear or branched alkoxy group, R^{2c} is chloride, and R^{2b}, R^{2d}, and R^{2e} are hydrogen.

Aspect 57. The compound of any one of Aspects 45 to 56, wherein the C1 to C10 substituted or unsubstituted linear or branched alkoxy group is a methoxy group.

Aspect 58. The compound of any one of Aspects 45 to 57, wherein R^{7a}, R^{7b}, R^{7c}, R^{7d}, R^{7e}, R^{7f}, R^{7g}, and R^{7h} are each hydrogen.

Aspect 59. The compound of Aspect 1, wherein the compound has the structure V or the pharmaceutically acceptable salt thereof

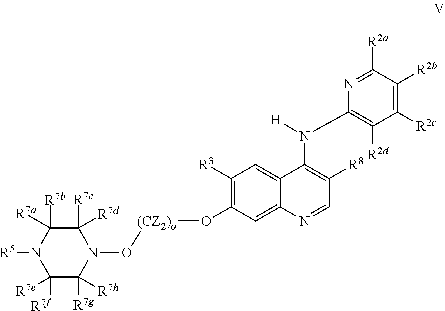

wherein
R^{2a}-R^{2d} are hydrogen, a halide, or an alkoxy group;
o is an integer from 1 to 5;
R³ is hydrogen, deuterium, a substituted or unsubstituted linear or branched alkyl group, a substituted or unsubstituted cycloalkyl group or heterocycloalkyl group, or a substituted or unsubstituted aryl group, and
R^{7a}, R^{7b}, R^{7c}, R^{7d}, R^{7e}, R^{7f}, R^{7g}, and R^{7h} are independently hydrogen, deuterium, or a substituted or unsubstituted alkyl group;
R⁸ is cyano; and
each Z is independently hydrogen or deuterium.

Aspect 60. The compound of Aspect 59, wherein R⁵ is a C1 to C5 alkyl group.

Aspect 61. The compound of Aspect 59, wherein R⁵ is a methyl group.

Aspect 62. The compound of any one of Aspects 59 to 61, wherein R³ is a C1 to C10 substituted or unsubstituted linear or branched alkoxy group.

Aspect 63. The compound of any one of Aspects 59 to 61, wherein R³ is a methoxy group.

Aspect 64. The compound of any one of Aspects 59 to 63, wherein o is an integer from 1 to 5.

Aspect 65. The compound of any one of Aspects 59 to 63, wherein o is 2.

Aspect 66. The compound of any one of Aspects 59 to 65, wherein R^{2a} is C1 to C10 substituted or unsubstituted linear or branched alkoxy group, R^{2b} is a halide, and R^{2c} and R^{2d} are hydrogen.

Aspect 67. The compound of any one of Aspects 59 to 66, wherein R^{7a}, R^{7b}, R^{7c}, R^{7d}, R^{7e}, R^{7f}, R^{7g}, and R^{7h} are each hydrogen.

Aspect 68. The compound of Aspect 1, wherein the compound has the structure VI or the pharmaceutically acceptable salt thereof

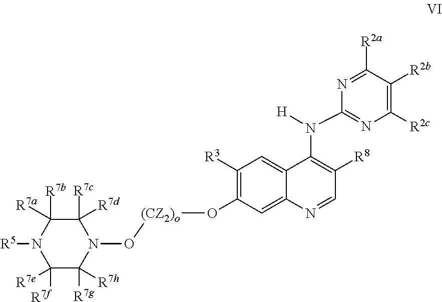

wherein
R^{2a}-R^{2c} are hydrogen, a halide, or an alkoxy group;
o is an integer from 1 to 5;
R³ is hydrogen, deuterium, a substituted or unsubstituted linear or branched alkyl group, a substituted or unsubstituted cycloalkyl group or heterocycloalkyl group, or a substituted or unsubstituted aryl group, and
R^{7a}, R^{7b}, R^{7c}, R^{7d}, R^{7e}, R^{7f}, R^{7g}, and R^{7h} are independently hydrogen, deuterium, or a substituted or unsubstituted alkyl group;
R⁸ is cyano; and
each Z is independently hydrogen or deuterium.

Aspect 69. The compound of Aspect 68, wherein R⁵ is a C1 to C5 alkyl group.

Aspect 70. The compound of Aspect 68, wherein R⁵ is a methyl group.

Aspect 71. The compound of any one of Aspects 68 to 70, wherein R³ is a C1 to C10 substituted or unsubstituted linear or branched alkoxy group.

Aspect 72. The compound of any one of Aspects 68 to 70, wherein R³ is a methoxy group.

Aspect 73. The compound of any one of Aspects 68 to 72, wherein o is an integer from 1 to 5.

Aspect 74. The compound of any one of Aspects 68 to 72, wherein o is 2.

Aspect 75. The compound of any one of Aspects 68 to 74, wherein R^{2a} is C1 to C10 substituted or unsubstituted linear or branched alkoxy group, R^{2b} is a halide, and R^{2c} is hydrogen.

Aspect 76. The compound of any one of Aspects 68 to 74, wherein R^{2a} is methoxy, R^{2b} is chloride, and R^{2c} is hydrogen.

Aspect 77. The compound of any one of Aspects 68 to 76, wherein R^{7a}, R^{7b}, R^{7c}, R^{7d}, R^{7e}, R^{7f}, R^{7g}, and R^{7h} are each hydrogen.

Aspect 78. The compound of Aspect 1, wherein the compound is

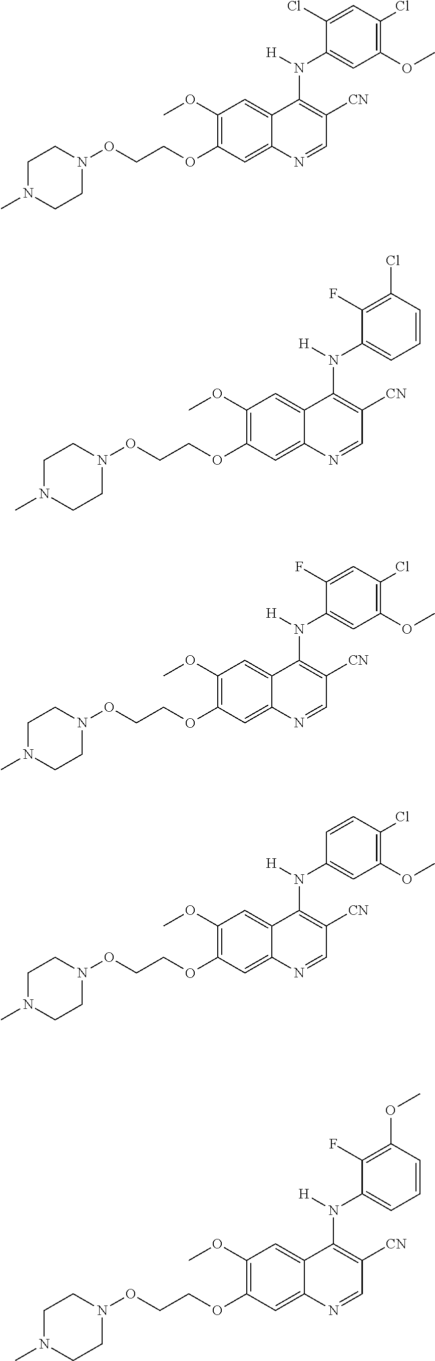

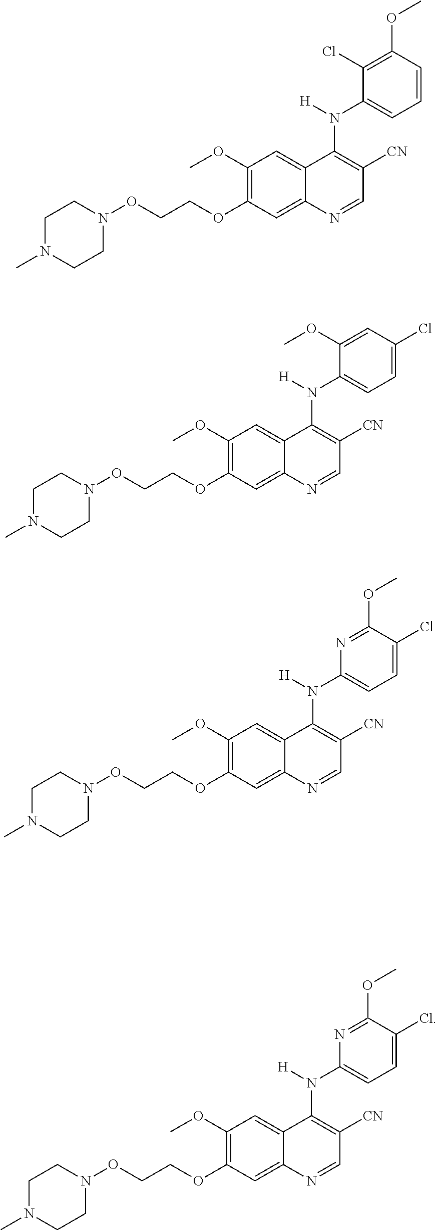

Aspect 79. A pharmaceutical composition comprising the compound of any one of claims 1 to 78 and a pharmaceutically-acceptable carrier.

Aspect 80. A method for treating a subject having chronic myeloid leukemia, acute lymphoblastic leukemia, glioblastoma, non-small cell lung cancer, prostate cancer, or colorectal cancer, the method comprising administering to the subject an effective amount of the compound of any one of Aspects 1 to 78.

Aspect 81. A method for inhibiting breakpoint cluster region, chromosome 22 (BCR) and Abelson proto-oncogene 1, chromosome 9 (ABL1) in a subject, the method comprising administering to the subject an effective amount of the compound in any one of Aspects 1 to 78.

Aspect 82. A method for treating a subject having a Philadelphia chromosome positive (Ph+) leukemia, the method comprising administering to the subject an effective amount of the compound of any one of Aspects 1 to 78.

Aspect 83. A method for evading P-gp-mediated efflux in a subject, the method comprising administering to the subject an effective amount of the compound of any one of Aspects 1 to 78.

Aspect 84. The method of any one of Aspects 80 to 83, wherein the compound is administered orally to the subject.

Aspect 85. The method of any one of Aspects 80 to 84, wherein the compound is administered at a dosage of from about 50 mg per day to about 1,000 mg per day.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure.

### Example 1

### General Synthesis Experimental for Examples 1 and 2

### General Experimental and Information

All reactions were conducted in single-neck, oven-dried glassware fitted with a rubber-septa under an argon atmosphere unless otherwise stated. All organic solutions were concentrated under reduced pressure on a rotary evaporator and water bath. Flash-column chromatography was performed using silica gel (Fisher Silica Gel Sorbent (230-400 Mesh, Grade 60)).³³ Thin-layer chromatography (TLC) was carried out with 250 μM glass back silica (XHL) plates with fluorescent indicator (254 nm). TLC plates were visualized by exposure to ultraviolet light (UV) and/or submersion in ceric ammonium molybdate (CAM) in ethanol followed by heating on a hot plate (120° C., 10-15 s). Solvents were purchased from Sigma-Aldrich and used without further purification.

4-Chloro-6-methoxy-7-(2-((4-methylpiperazin-1-yl)oxy)ethoxy)quinoline-3-carbonitrile (13) was prepared according to a literature procedure described in Reference 31 and had spectral data in accord with that previously reported. Pyridine hydrochloride was purchased from Sigma Aldrich. Palladium on carbon was purchased from Sigma Aldrich. 1-Chloro-5-fluoro-2-methoxy-4-nitrobenzene (23) was purchased from Sigma Aldrich. 6-Methoxypyridin-2-amine (24) was purchased from Ambeed. N-Chlorosuccinimide was purchased from Sigma Aldrich. Benzoyl chloride was purchased from Sigma Aldrich. 4-(Dimethylamino)pyridine was purchased from Sigma Aldrich. Pyridine was purchased from OakWood Chemicals. NMR spectra of all compounds were obtained in CDCl₃ (δ_{H} 7.26 and δ_{C} 77.16 ppm, respectively), or toluene-Dₐ(δ_{H} 7.09, 6.98, 7.00, 2.09 and δ_{C} 137.86, 129.24, 128.33, 125.49, 20.40 ppm, respectively) using a 500 MHz, EZC500 JEOL instrument at 298 K unless otherwise specified. The chemical shifts (6) are calculated with respect to residual solvent peak and are given in ppm. Multiplicities are abbreviated as followed: s (singlet), m (multiplet), b (broad), d (doublet), t (triplet), q (quartet).

High-resolution mass spectra (HRMS) were obtained on a ThermoFisher Orbitrap Q-Exactive instrument using electrospray ionization (ESI). UHPLC traces of compounds 7, 15-21 were obtained using a ThermoFisher Vanguish UHPLC with PDA detector and an Acclaim 120 ¹⁸C 4.6×50 mm column and the % purity determined using the Avalon peak area algorithm. Purities of all final compounds were confirmed to be >95% by UHPLC. The purity of commercial imatinib (1) and bosutinib (4) was confirmed by NMR and HRMS prior to in vitro study initiation.

### Biochemical Human RTK Kinase Enzymatic Radiometric Assay, KinaseProfiler (Independently Performed by Eurofins Cerep SA, FR)

Biochemical IC₅₀ determinations were performed using the well-established Eurofins Cerep KinaseProfiler technology at 10 μM ATP concentration, and a top testing concentration of 10 μM for all compounds. The IC₅₀ curve consisted of 9 test concentrations tested at half-log dilutions along with vehicle control wells.

### LoqD_{7.4} by Shake Flask Method (Independently Performed by Pharmaron Inc., Ninqbo, CN)

Test compounds and control compound (Nicardipine) were initially prepared as 10 mM solutions in DMSO and 5 μL of each sample solution was placed into one 96-well plate. After this, DMSO was added to dilute to a total volume of 50 μL yielding a working concentration of each compound of 1 mM. Then, 10 μL of the 1 mM working solution for each compound was placed into a new 96-well plate (log D plate) after which, 500 μL of saturated octanol was added into each vial of the log D plate followed by addition of 500 μL of saturated phosphate buffer (pH 7.4). The 96-well plate was then sealed and the plate transferred to the Eppendorf thermomixer comfort plate shaker and shaken at 25° C., 2,000 rpm for 2 h. After this, individual samples were centrifuged at 3,220 g at 25° C. for 30 min to separate phases and 100 μL was taken from the octanol and buffer phases and added to a new 96-well plate. From the octanol buffer phase, 5 μL was transferred to a new 96-well plate followed by addition of 495 μL of a 1:1 (v/v) mixture of H₂O and acetonitrile to yield 100-fold diluted samples. The diluted samples were then vortexed for 5 min at 1,000 rpm and 50 μL was transferred to a new 96-well plate, followed by addition of 450 μL of a 1:1 (v/v) mixture of H₂O and acetonitrile containing internal standard (100 nM alprazolam, 200 nM Caffeine and 100 nM tolbutamide) to yield 1,000-fold diluted samples. Diluted samples were then vortexed for 5 min at 1,000 rpm. 50 μL of buffer samples were then transferred to a new 96-well plate, followed by addition of 450 μL of a 1:1 (v/v) mixture of H₂O and acetonitrile mixture containing internal standard yielding 10-fold diluted buffer samples. Samples were vortexed for 5 min at 1,000 rpm and then all samples underwent analysis by LC-MS/MS.

### Solubility Determinations (Independently Performed by Pharmaron Inc., Ninqbo, CN)

Test compounds and control compounds (progesterone or diclofenac) were initially prepared as 10 mM DMSO stock solutions. Then, 15 μL of the stock solution and 485 μL of the solute were placed into a 96-well rack on the solubility sample plate. After addition, the solubility sample plate was transferred to the Eppendorf thermomixer comfort plate shaker and shook at 25° C., 1,100 rpm for 2 h. After such time, plugs and stir sticks were removed and the samples from the solubility sample plate were transferred into the filter plate. All the samples were then filtered and 5 μL of filtrate was taken and added along with 5 μL of DMSO and 490 μL of a 1:1 (v/v) mixture of H₂O and acetonitrile containing internal standard (100 nM alprazolam, 200 nM Caffeine and 100 nM tolbutamide) to the plate yielding a final concentration of 3 μM. The samples were then analyzed by LC-MS and diluted according to the peak shape with ultrapure water.

Filtrate was analyzed and quantified against a standard of known concentration using LC coupled with MS peak identification and quantitation. Solubility values of test compounds and progesterone were calculated as follows and are presented as the mean of n=2 replicates:

$$[ Sample ] = Area {ratio}_{sample} \times INJ \text{} VOL \text{} \text{} STD \times {DF}_{Sample} \times [ STD ] / Area ratio STD \times INJ VOL$$

### Metabolic Stability in Liver Microsomes (Independently Performed by Pharmaron Inc., Ninqbo, CN)

This experiment was carried out under two separate set of conditions: i) with Cofactors (NADPH), which consisted of adding 25 μL of 10 mM NADPH to the incubations, giving a final NADPH concentration of 1 mM, and ii) without Cofactors (NADPH), which consisted of adding 25 μL of 100 mM phosphate buffer to the incubations. Under both sets of conditions, the final concentration of microsomes were 0.5 mg/mL. Once the mixtures were prepared, the experiment was started by pre-warming the mixtures at 37° C. for 10 min. After such time, the reaction was initiated by adding 2.5 μL of control compound (verapamil) or test compounds prepared as 100 μM solutions, yielding final concentrations of 1 μM in the experiment. Solutions were then incubated in a water bath at 37° C. and aliquots of 30 μL were taken from the reaction solutions at 0.5, 15, 30, 45, and 60 min, at which times, the reaction was stopped by adding 150 μL of cold acetonitrile with internal standard (100 nM alprazolam, 200 nM caffeine and 100 nM tolbutamide). Samples were then centrifuged at 3,220 g for 40 min and aliquots of 100 μL of the supernatant were mixed with 100 μL of ultra-pure H₂O and analyzed by LC-MS/MS.

Peak areas were determined from extracted ion chromatograms. The slope value, k, was determined by linear regression of the natural logarithm of the remaining percentage of the parent drug vs incubation time curve.

The in-vitro half-life (in-vitro t_{1/2}) was determined from the slope value: in-vitro t_{1/2}=−(0.693/k)

Conversion of in-vitro t_{1/2} into the in-vitro intrinsic clearance (in-vitro Clᵢₙₜ in μL/min/mg protein) was done using the following equation (mean of n=2 replicates):

$$in - vitro {Cl}_{int} = ( 0.693 / {t}_{1 / 2} ) \times ( volume of incubation ( μL ) / amount of protein ( mg ) )$$

### Plasma Protein Binding by Equilibrium Dialysis (Independently Performed by Pharmaron Inc., Ninqbo, CN)

Initially, 1 mM solutions of test compounds and the control compound (Ketoconzaole) were prepared in DMSO. After this, dialysis membranes were soaked in ultrapure water for 60 min to separate strips, followed by 20% aq. ethanol for 20 min, and finally in a dialysis buffer for 20 min. The pre-soaked membranes were then loaded into the dialysis device and the air bath pre-heated to 37° C. After this, 597 μL of blank plasma solution followed by 3 μL of the working solutions of either test compounds or control compound were added to a new plastic plate or separate plastic tube and the solutions were vortexed at 1,000 rpm for 2 min. Immediately after this, 50 μL of spiked plasma solution suspension was transferred to a 96-well plate to act as T=0 control. The remaining samples were treated in the same manner, whereby all remaining spiked plasma solutions were added to the incubator for the duration of the study.

Cells were then loaded with 120 μL of the plasma samples and dialyzed against an equal volume of dialysis buffer (PBS). The unit was then covered with a gas permeable lid, incubated at 37° C., 5% CO₂ and shaken at 100 rpm for 6 h at 37° C. on an orbital shaker. At the end of incubation, the lid was removed and 50 μL of post-dialysis samples were removed from both buffer and plasma solution chambers into a separate 96-well plate for analysis. The plasma solutions (50 μL) were then added to the buffer samples along with an equal volume of PBS (50 μL) to the collected plasma solution samples. The plate was shaken at 1,000 rpm for 2 min and 400 μL of acetonitrile containing an appropriate internal standard was added to precipitate protein and release compound. The samples were then vortexed at 1,000 rpm for an additional 10 min followed by centrifugation at 3,220 g for 30 min. Supernatant (250 μL) was then added to a new 96-well plate and centrifuged again at 3,220 g for 30 min and 100 μL of this supernatant was then added to a new 96-well plate for analysis. The samples were analyzed by LC-MS/MS and 100 μL of distilled water was added to each sample prior to analysis.

Concentrations of test compounds and control compound in buffer and plasma solution chambers was determined and percentages of test compounds and control compound bound using the following equations (mean of n=2 replicates):

$$\% Unbound = ( Area {ratio}_{buffer chamber} / Area {ratio}_{plasma solution chamber} ) \times 100$$

$$\% Bound = 100 - \% Unbound$$

### Caco-2 Permeability (Independently Performed by Pharmaron Inc., Ningbo, CN)

To each well of the Transwell insert and reservoir were added 50 μL and 25 mL of cell culture medium, respectively. Following this, the HTS Transwell plates were incubated at 37° C., 5% CO₂ for 1 h prior to cell seeding. Caco-2 cells were then diluted with culture medium to to 6.86×10⁵ cells·mL⁻¹ and 50 μL of the cell suspension was dispensed into the filter well of the 96-well HTS Transwell plate. Cells were cultivated for 14-18 days in a cell culture incubator at 37° C., 5% CO₂, 95% relative humidity. The cell culture medium was replaced every two days, beginning no later than 24 h after initial plating. Once the cells had been prepared, test compounds and control compounds (Metoprolol and Digitoxin) were initially prepared as 2 mM DMSO solutions and were subsequently diluted with HBSS (10 mM HEPES, pH 7.4) to get 5 μM working solutions for test compounds, and 10 μM for control compounds. Following this, the Caco-2 plate was removed from the incubator and the monolayer washed twice with pre-warmed HBSS (10 mM HEPES, pH 7.4). The plate was then incubated at 37° C. for 30 min.

To determine the rate of drug transport in the apical to basolateral direction, 125 μL of the working solution of either test compounds or control compounds was added to the Transwell insert (apical compartment) and a 50 μL sample was immediately transported from the apical compartment to 200 μL of acetonitrile containing internal standard (100 nM alprazolam, 200 nM Caffeine and 100 nM tolbutamide) in a new 96-well plate as the initial donor sample (A-B). The sample was then vortexed at 1,000 rpm for 10 min. The receiver plate (basolateral compartment) wells were then filled with 235 μL of transport buffer. To determine the rate of drug transport in the basolateral to apical direction, 285 μL of the working solution of either test compounds or control compounds was added to the receiver plate wells (basolateral compartment) and a 50 μL sample was immediately transferred from the basolateral compartment to 200 μL of acetonitrile containing internal standard in a new 96-well plate as the initial donor sample (B-A). The sample was vortexed at 1,000 rpm for 10 min. The Transwell insert (apical compartment) was then filled with 75 μL of transport buffer and the apical to basolateral direction and the basolateral to apical direction were done at the same time. The plates were then incubated at 37° C. for 2 h. After incubation, 50 μL samples from donor sides (apical compartment for Ap→BI flux, and basolateral compartment for BI→Ap) and receiver sides (basolateral compartment for Ap→BI flux, and apical compartment for BI→Ap) were transferred to wells of a new 96-well plate, followed by the addition of 200 μL acetonitrile containing internal standard. All samples were vortexed for 10 min and then centrifuged at 3,220 g for 40 min. A 150 μL aliquot of the supernatant was then mixed with an appropriate volume of ultra-pure water prior to LC-MS/MS analysis.

To monitor for monolayer integrity, Lucifer yellow leakage was determined after the 2 h transport period. This began by preparing a stock solution of Lucifer yellow in DMSO which was diluted with HBSS (10 mM HEPES, pH 7.4.) to reach a final concentration of 100 μM. To each Transwell insert (apical compartment) was then added 100 μL of the Lucifer yellow solution, followed by filling the wells in the receiver plate (basolateral compartment) with 300 μL of HBSS (10 mM HEPES, pH 7.4). The plates were then incubated at 37° C. for 30 min and 80 μL samples were removed directly from the apical and basolateral wells (using the basolateral access holes) and transferred to new 96 well plates. The Lucifer yellow fluorescence signal was measured in a fluorescence plate reader at 480 nM excitation and 530 nM emission.

The apparent permeability (Pₐₚₚ), in units (cm·s⁻¹×10⁻⁶), was calculated for drug transport assay using the following equation:

$${P}_{app} = ( {V}_{A} \times {[ drug ]}_{acceptor} ) / ( Area \times Time \times {[ drug ]}_{initial , donor} )$$

where, V_{A} represents the volume (mL) in the acceptor well. Area is the surface of the membrane (0.143 cm² for Transwell-96 well permeable supports) and time is the total transport time in seconds.

The efflux ratio was determined using the following equation:

$$Efflux Ratio = {P}_{app ( B - A )} / {P}_{app ( A - B )}$$

where, P_{app(B-A)} indicates the apparent permeability coefficient in the basolateral to apical direction, and P_{app (A-B)} indicates the apparent permeability coefficient in the apical to basolateral direction.

Lucifer yellow leakage of monolayer was calculated using the following equation:

$$LY Leakage = ( {I}_{acceptor} \times 0.3 / {I}_{acceptor} \times 0.3 + {I}_{donor} \times 0.1 ) \times 100 \%$$

where, I_{acceptor} is the fluorescence intensity in the acceptor well (0.3 mL), and I_{donor} is the fluorescence intensity in the donor well (0.1 mL) and expressed as % leakage. All Lucifer yellow percentage amount transported values were found to be less than 0.5% in the study.

### MDCKII-MDR1 Permeability (Independently Performed By: Pharmaron Inc., Ninqbo, CN)

To each well of the Transwell insert and reservoir were added 50 μL and 25 mL of cell culture medium, respectively. Following this, the HTS Transwell plates were incubated at 37° C., 5% CO₂ for 1 h prior to cell seeding. MDCKII-MDR1 cells were diluted with culture medium to 1.56×10⁶ cells·mL⁻¹ and 50 μL of cell suspension was dispensed into the filter well of the 96-well HTS Transwell plate. Cells were cultivated for 4-8 days in a cell culture incubator at 37° C., 5% CO₂, 95% relative humidity. The cell culture medium was replaced every other day, beginning no later than 24 h after initial plating. Once the cells had been prepared, test compounds and control compounds (Metoprolol and Digitoxin) were initially prepared as 200 μM DMSO solutions and were subsequently diluted with HBSS (10 mM HEPES, pH 7.4) to get 1 μM working solutions.

Transepithelial electrical resistance (TEER) across the monolayer was measured using Millicell Epithelial Volt-Ohm measuring system and the plate was returned to the incubator once the measurement was done.

The TEER value was calculated according to the following equation:

$$TEER measurement ( ohms ) \times Area of membrane ( {cm}^{2} ) = TEER value ( ohm \cdot {cm}^{2} )$$

The TEER value was greater than 42 ohm-cm², indicative of a well-qualified MDCKII-MDR1 monolayer.

The MDCKII-MDR1 plate was then removed from the incubator and washed twice with pre-warmed HBSS (10 mM HEPES, pH 7.4) followed by incubation at 37° C. for 30 min.

To determine the rate of drug transport in the apical to basolateral direction, 125 μL of the working solution of either test compounds or control compounds was added to the Transwell insert (apical compartment) and a 50 μL sample was immediately transferred from the apical compartment to 200 μL of acetonitrile containing internal standard (100 nM alprazolam, 200 nM caffeine, and 100 nM tolbutamide) in a new 96-well plate as the initial donor sample (A-B). The sample was then vortexed at 1,000 rpm for 10 min. The receiver plate (basolateral compartment) wells were then filled with 235 μL transport buffer. To determine the rate of drug transport in the basolateral to apical direction, 285 μL of the working solution of either test compounds or control compounds was added to the receiver plate (basolateral compartment) wells and a 50 μL of sample was immediately transferred from the basolateral compartment to 200 μL of acetonitrile containing internal standard in a new 96-well plate as the initial donor sample (B-A). The sample was vortexed for 10 min at 1,000 rpm. The Transwell insert (apical compartment) was then filled with 75 μL of transport buffer and the apical to basolateral direction and the basolateral to apical direction were done at the same time. The plates were incubated at 37° C. for 2 h. After incubation, 50 μL samples from donor sides (apical compartment for Ap→BI flux, and basolateral compartment BI→Ap) and receiver sides (basolateral compartment for Ap→BI flux, and apical compartment for BI→Ap) were transferred to wells of a new 96-well plate, followed by the addition of 200 μL acetonitrile containing internal standard. The samples were vortexed for 10 min and then centrifuged at 3,220 for 40 min. An aliquot of 100 μL of the supernatant was mixed with an appropriate volume of ultra-pure water before LC-MS/MS analysis.

To monitor for monolayer integrity, Lucifer yellow leakage was determined after the 2 h transport period. This began by preparing a stock solution of Lucifer yellow in DMSO and diluted with HBSS (10 mM HEPES, pH 7.4.) to reach a final concentration of 100 μM. To each Transwell insert (apical compartment) was then added 100 μL of the Lucifer yellow solution, followed by filling the wells in the receiver plate (basolateral compartment) with 300 μL of HBSS (10 mM HEPES, pH 7.4). The plates were then incubated at 37° C. for 30 min and 80 μL samples were removed directly from the apical and basolateral wells (using the basolateral access holes) and transferred to new 96 well plates. The Lucifer yellow fluorescence signal was measured in a fluorescence plate reader at 480 nM excitation and 530 nM emission.

The apparent permeability coefficient (Pₐₚₚ) in units (cm·s⁻¹×10⁻⁶), can be calculated for MDCKII-MDR1 drug transport assays using the following equation:

$${P}_{app} = ( {V}_{A} \times {[ drug ]}_{acceptor} ) / ( Area \times Time \times {[ drug ]}_{initial , donor} )$$

where, V_{A} represents the volume (mL) in the acceptor well. Area is the surface of the membrane (0.143 cm² for Transwell-96 Well Permeable Supports) and time is the total transport time in seconds.

The efflux ratio was determined using the following equation:

$$Efflux Ratio = {P}_{app ( B - A )} / {P}_{app ( A - B )}$$

where, P_{app(B-A)} indicates the apparent permeability coefficient in the basolateral to apical direction, and P_{app (A-B)} indicates the apparent permeability coefficient in the apical to basolateral direction.

Lucifer yellow leakage of monolayer was calculated using the following equation:

$$\% LY leakage = 100 \times {[ LY ]}_{acceptor} / ( {[ LY ]}_{donor} + {[ LY ]}_{acceptor} )$$

All Lucifer yellow percentage amount transported values were found to be less than 0.5% in the study.

### Metabolic Stability in Hepatocytes (Independently Performed by Pharmaron Inc., Ninqbo, CN)

Initially, test compounds and the control compound (Verapamil) were prepared as 10 mM solutions in DMSO. In separate conical tubes, 100 μM solutions of test compounds and the control compound (Verapamil) were prepared by addition of 2 μL of the 10 mM stock solutions to 198 μL of 1:1 (v/v) acetonitrile:water.

Incubation medium (William's E Medium supplemented with GlutaMAX) and hepatocyte thawing medium were then placed in a 37° C. water bath and allowed to warm for at least 15 min prior to use. A vial of cryopreserved hepatocytes was then thawed by placing the vial in a 37° C. water bath and gently shaking for 2 min. After thawing was complete, hepatocytes were transferred into a 50 mL conical tube containing thawing medium and the tube was then placed into a centrifuge and spun at 100 g for 10 min. Upon completion, the thawing medium was aspirated, and hepatocytes resuspended in enough incubation medium to yield ~1.5×10⁶ cells/mL. Using AO/PI staining, cells were counted to determine the viable cell density. Cells were then diluted with incubation medium to yield a working cell density of 0.5×10⁶ viable cells/mL, after which, 198 μL of hepatocytes were pipetted into each well of a 96-well non-coated plate and the plate was placed in the incubator to allow the hepatocytes to warm at 37° C. for 10 min. Subsequently, 2 μL of the 100 μM test compounds or control compound solutions was transferred into the respective wells of the 96-well non-coated plate to start the reaction. The plate was returned to the incubator for 0.5, 15, 30, 60, 90, and 120 min, at which times, 25 μL of the well contents were transferred to be mixed with 150 μL of acetonitrile containing internal standard (100 nM alprazolam, 200 nM caffeine and 100 nM tolbutamide) to terminate the reaction. Samples were vortexed for 5 min and centrifuged at 3,220 g for 45 min. Aliquots of 100 μL of the supernatant were then diluted by 100 μL of ultra-pure water, and the mixture was analyzed by LC-MS/MS.

All calculations were carried out using Microsoft Excel. Peak areas were determined from extracted ion chromatograms. The in-vitro half-life (t_{1/2}) of parent compound was determined by regression of the percent parent disappearance vs. time curve.

The in-vitro half-life (in-vitro t_{1/2}) was determined from the slope value: in-vitro t_{1/2}=−(0.693/k)

Conversion of the in-vitro half-life (t_{1/2}) (in min) into the in-vitro intrinsic clearance (in-vitro Clᵢₙₜ in μL/min/1×10⁶ cells) was done using the following equation (mean of n=2 replicates):

$$in - vitro {Cl}_{int} = - kV / N$$

where, N=number of hepatocytes per well (0.1×10⁶ cells)
   hERG Safety Evaluation by Manual Patch-Clamp System (Independently Performed by Pharmaron Inc., Ningbo, CN)

The HEK 293 cell line, which contains hERG stably expressed, was used in this experiment, and was cultured in 85% DMEM, 10% dialyzed FBS, 0.1 mM NEAA, 25 mM HEPES, 100 Units/mL Penicillin-Streptomycin, 5 μg/mL Blasticidin and 400 μg/mL Geneticin. Cells were split using TrypLE™ Express about three times a week, and maintained between ~40% to 80% confluence. Before the assay, the cells were placed onto the coverslips at 5×10⁵ cells per 6 cm cell culture dish and induced with doxycycline at 1 μg/mL for 48 h.

Extracellular solution (in mM) was prepared as follows: 132 NaCl, 4 KCl, 3 CaCl₂, 0.5 MgCl₂, 11.1 glucose, and 10 HEPES (pH adjusted to 7.35 with NaOH). Intercellular solution (in mM) was prepared as follows: 140 KCl, 2 MgCl₂, 10 EGTA, 10 HEPES and 5 MgATP (pH adjusted to 7.35 with KOH). Test compounds were prepared initially as a 10 mM stock solution in DMSO and then the stock solution of each compound was serial-diluted by a ratio of 1:3 with DMSO to prepare an additional 3 intermediary solutions of 3.33, 1.11 and 0.37 mM, respectively. Before the experiment, the working solutions were finally prepared by 1000-fold dilutions of the above-described intermediary solutions using extracellular solution, while 30 μM working solution was prepared by 333.333-fold dilution of the 10 mM DMSO solution. hERG current in the presence of 5 doses (0.37, 1.11, 3.33, 10, 30 μM) were tested for IC₅₀ determination.

The coverslip was removed from the cell culture dish and placed on the microscope stage in the bath chamber and the desirable cell was located using the ×10 objective. Tip of the electrode was located under the microscope using the ×10 objective by focusing above the plane of the cells. Once the tip was in focus, the electrode was advanced downwards towards the cell using the coarse controls of the manipulator, while simultaneously moving the objective to keep the tip in focus. When directly over the cell, the magnification was switched to ×40 objective and fine controls of the manipulator were used to approach the surface of the cell. Gentle suction was then applied through the side-port of the electrode holder to form a gigaOhm seal. C_{fast} was then used to remove the capacity current in coincidence with the voltage step. Whole cell configuration was obtained by applying repetitive, brief, strong suction until the membrane patch ruptured. Membrane potential was set to −60 mV to ensure that the hERG channels were not open. Spikes of the capacity current were then cancelled using C_{slow} on the amplifier. Holding potential was set to −90 mV for 500 ms recorded current at 20 kHz and filtered at 10 kHz. Leaking current was tested at −80 mV for 500 ms. hERG current was elicited by depolarizing at +30 mV for 4.8 sec and then voltage was taken back to −50 mV for 5.2 seconds to remove the inactivation and observe the deactivating tail current. The maximum amount of tail current size was used to determine hERG current amplitude. Current was then recorded for 120 seconds to assess the current stability and only stable cells with recording parameters above the thresholds were applied for the compound administration. First vehicle control was applied to the cells to establish the baseline. Once the hERG current was found to be stabilized for 5 min, working solutions were applied. hERG current in the presence of test compounds were recorded for approximately 5 min to reach steady state and then 5 sweeps were captured. For dose response testing, 5 doses of test compounds were applied to the cells cumulatively from low to high concentrations (0.37, 1.11, 3.33, 10, 30 μM). The positive control article, dofetilide at 150 nM was also applied to each cell post hERG current measurement at highest concentration of test compounds as the internal low control for normalization of percentage inhibition. In order to ensure the proper performance of the culture cells and operations, the positive control Dofetilide, with 5 doses was also used to test the same batch of cells.

Data were deemed acceptable if: initial seal resistance >1 GΩ; Leak currents <50% of the control peak tail currents at any time; Peak tail amplitude >250 pA; Membrane resistance Rm>500 MΩ; Access resistance (Ra)<10 MΩ; Apparent run-down of peak current <2.5% per min. Data that met the aforementioned criteria were further analyzed. Percent current inhibition was calculated using the following equation (n=3 determinations):

$$Peak current inhibition = ( 1 - Peak tail {current}_{compound} - Peak tail {current}_{positive control} / Peak tail {current}_{blank vehicle} - Peak tail {current}_{positive control} ) \times 100$$

The dose response curve of test compounds was plotted with % inhibition against the concentration of test compounds using Graphpad Prism 9.5.1. IC₅₀ was calculated with a non-linear fit [inhibitor] vs normalized response with sigmoidal, X is concentration graph on GraphPad Prism version 9.5.1. For further information on statistical analysis please see Statistical Analysis.

### CYP Inhibition of CYP1A2, CYP2C9, CYP2C19, CYP2D6 and CYP3A4 in Human Liver Microsomes (Independently Performed by Pharmaron Inc., Ninqbo, CN)

Serial dilutions were performed to obtain solutions of each compound at the nominal concentrations of 6000, 2000, 600, 200, 60, 20, 6 and 2 (μM) in DMSO. The final concentrations of the test compound(s) were: 30, 10, 3, 1, 0.3, 0.1, 0.03, 0.01 μM.

Incubation was initially carried out in 96 well plates and the following volumes were dispensed into each well of the incubation plate: 169 μL of the master solution and 1 μL of the test compound working solution or vehicle (acetonitrile). The incubation plate was then placed into the water bath and pre-warmed at 37° C. for 5 min. Subsequently 10 μL of substrate solution was added to the incubation plate and the plate was mixed on a whirly mixer for 15 seconds, and then 20 μL of a 10 mM NADPH solution was added to start the reaction at the final concentration of 1 mM. The assay was performed in duplicate.

The reaction was then quenched by addition of 300 μL of cold acetonitrile containing 3% formic acid and 200 nM tolbutamide, 200 nM aprozolam and 200 nM labalol. The plate was centrifuged at 3,220 g for 40 min. Next, 150 μL of the supernatant was added to a new plate, and the supernatant was also diluted with 150 μL of pure water if necessary. The contents were mixed well and the samples analyzed by UPLC-MS/MS.

The automatic peak integration areas were checked for all samples. The inhibition of each P450 enzyme in human liver microsomes was measured as the percentage decrease in the activity of marker metabolite formation compared to non-inhibited controls (=100% activity). The IC₅₀ values are calculated with remaining activity (%) and logarithm of inhibitor concentrations. IC₅₀ was calculated with a non-linear fit [inhibitor] vs normalized response with variable slope on GraphPad Prism version 9.5.1. For further information on statistical analysis please see Statistical Analysis.

Remaining activity was calculated as follows:

$$Area ratio = Peak {area}_{( Analyte )} / Peak {area}_{( Internal Standard )}$$

$$Remaining activity ( \% ) = Area {ratio}_{( Test compound )} / Area {ratio}_{( vehicle )} \times 100 \%$$

### Direct and Time-Dependent Inhibition of CYP2D6 and CYP2C19 in Human Liver Microsomes by Compound 7 (Independently Performed by: Pharmaron Inc., Ninqbo, CN)

Initially, a 30 mM stock solution of the test compound (7) was prepared in DMSO. The stock solution was then diluted to 0.012, 0.04, 0.12, 0.4, 1.2, 4 mM with DMSO. Final concentration of test compound (7) was 0.06, 0.2, 0.6, 2, 6, 20 μM. Positive control compounds (Paroxetine for CYP2D6 and Fluoxetine for CYP2C19) were prepared as follows:

Concentration

| | | Working | Final |
|---|---|---|---|
| CYP | Positive | Concentration | Concentration |
| Isoform | Control | (μM) | (μM) |
| CYP2D6 | Paroxetine | 0.006, 0.02, 0.06, | 0.03, 0.1, 0.3, |
| | | 0.2, 0.6, 2 | 1, 3, 10 |
| CYP2C19 | Fluoxetine | 0.06, 0.2, 0.6, 2, | 0.3, 1, 3, 10, |
| | | 6, 20 | 30, 100 |

| | | Working | Final | |
|---|---|---|---|---|
| CYP | | Concentration | Concentration | Incubation |
| Isoform | Substrate | (μM) | (μM) | Time |
| CYP2D6 | Bufuralol | 40 | 2 | 20 min |
| CYP2C19 | Mephenytoin | 1000 | 50 | 20 min |

The incubation was carried out in 96 well plates and the following volumes were dispensed into each of the wells of the plate: 169 μL of master solution and 1 μL of test compound or control compound at the respective final concentrations. The incubation plate was then placed into the water bath and pre-warmed at 37° C. for 5 min. This experiment was performed in duplicate.

For the 0 min pre-incubation experiment, 10 μL of substrate (details given in the table above) was added to the incubation plate and then 20 μL of 10 mM NADPH solution was added to start the reaction at a final concentration of 1 mM.

For the 30 min pre-incubation with NADPH experiment, 20 μL of 10 mM NADPH solution was added to the incubation plate at a final concentration of 1 mM and then pre-incubated in the 37° C. water bath for 30 min. After 30 min incubation, 10 μL of substrate was added to start the reaction.

For the 30 min pre-incubation without NADPH experiment, the incubation plate was pre-incubated in a water bath at 37° C. for 30 min. After 30 min, 10 μL of substrate and then 20 μL of 10 mM NADPH solution was added to start the reaction at a final concentration of 1 mM.

Reactions were quenched at the specified times by addition of 300 μL of cold acetonitrile with 3% formic acid, 200 nM alprazolam, 200 nM labetalol and 200 nM tolbutamide. The plate was then centrifuged at 3,220 for 50 min at 4° C. and 150 μL of the supernatant was placed into a new plate. If needed, the supernatant was diluted with 150 μL pure water and the contents were mixed well and analyzed by UPLC-MS/MS.

The automatic peak integration areas were checked for all samples. The inhibition of each P450 enzyme in human liver microsomes was measured as the percentage decrease in the activity of marker metabolite formation compared to non-inhibited controls (=100% activity). The IC₅₀ values are calculated with remaining activity (%) and logarithm of inhibitor concentrations. IC₅₀ was calculated with a non-linear fit [inhibitor] vs normalized response with variable slope on GraphPad Prism version 9.5.1. For further information on statistical analysis please see Statistical Analysis.

Remaining activity was calculated as follows:

$$Area ratio = Peak {area}_{( Analyte )} / Peak {area}_{( Internal Standard )}$$

$$Remaining activity ( \% ) = Area {ratio}_{( Test compound )} / Area {ratio}_{( vehicle )} \times 100 \%$$

### AMES Fluctuation Test (Independently Performed by Eurofins Panlabs, St Charles, MO, USA)

Test compound (7) and control compounds (2-aminoanthracene, 9-aminoacridine, quercetin, streptozotocin) were tested at 5, 10, 50, 100 μM with n=48 wells. Quercetin is the positive control for frameshift mutation (TA98), and streptozotocin for base pair insertions/deletions (TA100 and TA1535). Aminacridine is the positive control for frameshift mutations (TA1537). 2-Aminoanthracene requires metabolic activation, thereby ensuring the quality of the S9 fraction. A bacterial cytotoxicity test was conducted in parallel at 8 concentrations (with 100 μM as the highest concentration) with n=3 wells. The assay was run with and without metabolic activation by using rat liver S9 fraction. Four Salmonella strains were used as summarized below. Respective reference compounds were tested concurrently with test compounds and the data was compared with historical values determined at Eurofins.

| Salmonella | DNA | His | Reversion |
|---|---|---|---|
| Strain | Target | Mutation | Event |
| TA98 | CGCGCGCG | hisD3052 | Frameshifts |
| TA100 | GGG | hisG46 | Base-pair |
| | | | substitution |
| TA1535 | GGG | hisG46 | Base-pair |
| | | | substitution |
| TA1537 | Near CCC | hisC3076 | Frameshifts |
| | run | | |

### Micronucleus Test (Independently Performed by Eurofins Panlabs, St Charles, MO, USA)

Test compound (7) was assayed at 0.2, 0.5, 2, 5, 20, 50, 200, 500 μM and control compounds (cyclophosphamide, mitomycin C) were tested at 36 and 0.3 μM, respectively, in Chinese hamster ovary (CHO)—K1 cell lines both with and without metabolic activation by rat liver S9. High content analysis and fluorescent image analysis were used to detect micronuclei. Cyclophosphamide was the positive control in the presence of metabolic activation by rat liver S9 and mitomycin C was the positive control without metabolic activation by rat liver S9. Respective reference compounds were tested concurrently with test compounds and the data was compared with historical values determined at Eurofins.

### In Vitro CTG Assay (Independently Performed By: Crown Bioscience, Taicanq, CN)

| Cell | | | | Compound |
|---|---|---|---|---|
| Line | Tissue | Culture | | Treatment |
| Name | Origin | Property | Cell Culture Medium | Time |
| MEG- | Blood | Adherent | RPMI1640 + 10% FBS | 72 h |
| 01 | | and | | |
| | | Suspension | | |
| Molt-4 | Blood | Suspension | RPMI1640 + 10% FBS | 72 h |
| SUP- | Bone | Suspension | IMDM + 20% FBS + 0.05 mM | 72 h |
| B15 | Marrow | | β-ME | |
| KU812 | Peripheral | Suspension | RPMI1640 + 10% FBS | 72 h |
| | Blood | | | |

Cells listed in the table above were initially harvested during logarithmic growth period followed by re-suspension and counting using a Vi cell counter (cell viability was measured by trypan blue exclusion assay). Cells were then diluted and 90 μL cell suspensions were added to 96-well plates. Plates were then incubated overnight in a humidified incubator at 37° C. with 5% CO₂. Tₒ(Time 0) plate was then taken by 10 μL of culture medium being added to each well followed by 50 μL CellTiter-Glo® Reagent being added. The contents were then mixed for 2 min on an orbital shake to facilitate cell lysis. Next, the plate was incubated at room temperature for 10 min to stabilize luminescent signal. Blackseal black sticker was placed to the bottom of each plate and the luminescence was recorded using EnVision multi label reader. Test compounds and 10 μL of each 10× compound working solutions was added according to the plate inoculation map. Plates were then placed back into the incubator and after 72 h, 50 μL CellTiter-Glo® was added to each well. Contents were then mixed for 2 min on an orbital shaker to facilitate cell lysis. The plate then was incubated at room temperature for 10 min to stabilize luminescent signal. Blackseal black sticker was placed to the bottom of each plate and the luminescence recorded using EnVision multi label reader.

Relative IC₅₀ was calculated with a dose-response curve fitted using nonlinear regression model with a sigmoidal dose response on GraphPad Prism version 9.5.1. For further information on statistical analysis please see Statistical Analysis. The formula for calculating survival rate was (n=3 replicates):

$$Surviving rate ( \% ) = ( {Lum}_{Test Compound} - {Lum}_{Medium control} ) / {Lum}_{None treated} - {Lum}_{Medium control} ) \times 100 \%$$

### Animal Use (All Animal Studies Independently Performed by Pharmaron Inc., Beijinq, CN)

Animal experiments were performed at Pharmaron Inc., Beijing, CN and animal use was approved by Pharmaron's Institutional Animal Care and Use Committee (IACUC) (Pharmaron IACUC, Protocol #PK-M-07182022). Study protocols were in accordance with the IACUC of the University of Georgia and conformed to the standards established by NIH guidelines on animal welfare, Chinese legislation and Pharmaron's IACUC policies and procedures. Six- to eight-week-old male CD1 mice (approximately 20-30 g) were used in the pharmacokinetic studies, n=3 mice per route.

### Pharmacokinetic Study in Male CD1 Mice (Independently Performed by Pharmaron Inc., Beijing, CN)

Compound 7 was administered by IV (formulation: DMSO: 10% captisol in saline=1:99) via tail vein and PO (formulation: 0.5% CMC, 2.0% Tween 80, 0.06% acetic acid in water) via oral gavage followed by blood sampling at 8 time points for IV (0.0833, 0.25, 0.5, 1, 2, 4, 7, 24 hours post dose) and 7 time points for PO (0.25, 0.5, 1, 2, 4, 8, 24 hours post dose). Approximately 0.03 mL of blood was collected from dorsal metatarsal vein at each time point. Blood at each sampling point was transferred into a plastic micro centrifuge tube containing K₂-EDTA and collection tubes with blood samples and anticoagulant were inverted several times for proper mixing of the tube contents and placed on wet ice prior to centrifugation for plasma. Blood samples were centrifuged at 4,000 g for 5 min at 4° C. to obtain plasma. Samples were stored in a freezer at −75° C. prior to analysis. Concentrations of test articles in the plasma samples were analyzed using LC-MS/MS method. WinNonlin (Phoenix™) or other similar software was used for pharmacokinetic calculations.

### Statistical Analysis

Statistical analysis was performed using GraphPad Prism software unless otherwise stated. Data are presented as mean or median±SD or SEM as indicated when n=≥3, or as the mean when n=2. For in vitro ADME studies, data is presented as mean of n=2 biological replicates unless otherwise stated. For in vivo PK experiments, data is presented as mean±SD, n=3 animals per study arm (intravenous (IV) or oral gavage (PO)). For in vitro short-term growth delay experiments, IC₅₀ values were determined from the nonlinear regression equation with a sigmoidal dose response and are presented as the mean (95% confidence interval), n=3 biological replicates.

### SYNTHESIS

### Synthesis of 1-(2-((tert-butyldimethylsilyl)oxy)ethoxy)-4-methylpiperazine (10) by Direct N—O Bond Formation³⁴

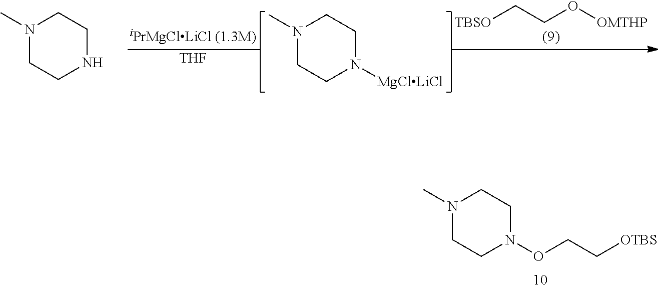

To a stirred solution of N-methylpiperazine (8) (6.31 mL, 56.9 mmol, 3.0 eq.) in anhydrous THF (47.5 mL) at 0° C. was added ⁱPrMgCI·LiCl (1.3M in THF) (36.4 mL, 47.4 mmol, 2.5 eq.) after which the solution was brought to r.t. and stirred for 45 min.* After such time, 9³⁵ (5.5 g, 18.95 mmol, 1.0 eq.) was added dropwise in anhydrous THF (47.5 mL) and the solution was stirred for 3 h. Next, the solution was quenched with NaHCO₃ (100 mL). The layers were separated and the aqueous layer extracted with EtOAc (3×75 mL). The combined organic layers were dried over Na₂SO₄, filtered and concentrated in-vacuo. The residue obtained was purified by flash column chromatography on silica (eluent: 40:55:5 hexanes:EtOAc:Et₃N) to afford the title compound 10 (3.26 g, 11.89 mmol, 62%).

Physical State: Light yellow oil.

TLC R_{f}=0.30 (40:55:5 Hexanes:EtOAc:Et₃N; CAM)

¹H NMR (500 MHz, toluene-D₈): δ 3.77-3.75 (m, 2H), 3.72-3.70 (m, 2H), 3.09 (br d, J=10.0 Hz, 2H), 2.76 (br s, 2H), 2.46 (br d, J=11.0 Hz, 2H), 2.07-2.02 (m, 5H). 0.97 (s, 9H), 0.07 (s, 6H).

¹³C NMR (126 MHz, Toluene-D₈): δ 73.1, 62.0, 55.8, 54.5, 45.5, 26.1, 18.5, −5.2.

HRMS-ESI (m/z): [M+H]⁺ calculated for [C₁₃H₃₁N₂O₂²⁸Si]⁺: 275.2155, found: 275.2144.

*Magnesium amide generation evolves propane (g). Caution should be exercised on larger scale.

### Synthesis of 2-((4-methylpiperazin-1-yl)oxy)ethan-1-ol (11)

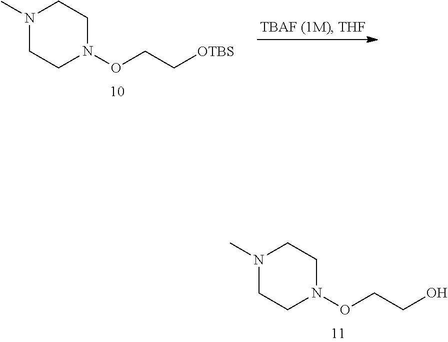 [SMILES: CN1CCN(OCCO[Si](C)(C)C(C)(C)C)CC1 ; CN1CCN(OCCO)CC1]

To a stirred solution of 10 (3.20 g, 11.67 mmol, 1.0 eq.) in anhydrous THF (140 mL) at 0° C. was added TBAF (1M in THF) (23.34 mL, 23.34 mmol, 2.0 eq.) slowly, after which, the solution was brought to r.t. and stirred for 1 h. After such time, the mixture was concentrated in-vacuo and the residue obtained was purified by flash column chromatography (eluent: 90:10 EtOAc:Et₃N) to afford the title compound 11 (1.21 g, 7.56 mmol, 65%).

Physical State: Yellow oil.

TLC R_{f}=0.10 (90:10 EtOAc:Et₃N; CAM)

¹H NMR (500 MHz, Toluene-D₈): δ 3.68-3.66 (m, 2H), 3.65-3.61 (m, 2H), 3.01 (br d, J=10.3 Hz, 2H), 2.69-2.64 (m, 2H), 2.40 (br d, J=11.4 Hz, 2H), 1.96 (s, 3H), 1.94-1.89 (m, 2H).

¹³C NMR (126 MHz, Toluene-D₈): δ 72.2, 63.2, 55.4, 54.2, 45.3.

HRMS-ESI (m/z): [M+H]⁺ calculated for [C₇H₁₇N₂O₂]⁺:161.1290, found: 161.1282.

### Synthesis of 4-chloro-2-fluoro-5-methoxyaniline

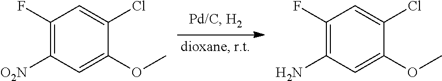

A stirred solution of 1-chloro-5-fluoro-2-methoxy-4-nitrobenzene (2.5 g, 12.16 mmol, 1.0 eq.) in dioxane at r.t. was treated with Pd/C (10% by wt) (250 mg), placed under 1 atm of H₂ (balloon) by repeated evacuation and back filling with H₂ and was stirred for 20 h at r.t. After such time, the mixture was filtered over Celite® and the solid washed with EtOAc (50 mL). The resulting filtrate was concentrated and the residue obtained was purified by flash column chromatography on silica (eluent: 25:75 EtOAc:Hexanes) to afford the title compound (1.82 g, 10.40 mmol, 85%).

Physical State: Light purple solid.

TLC R_{f}=0.10 (25:75 EtOAc:Hexanes; UV, CAM)

¹H NMR (500 MHz, CDCl₃): δ 7.01 (d, J=10.3 Hz, 1H), 6.35 (d, J=8.0 Hz, 1H), 3.80 (s, 3H), 3.73 (br s, 2H).

¹³C NMR (126 MHz, CDCl₃): δ 151.9 (d, ⁴J_{C-F}=2.5 Hz), 145.4 (d, ¹J_{C-F}=235.6 Hz), 133.9 (d, ²J_{C-F}=13.9 Hz), 117.0 (d, ²J_{C-F}=23.9 Hz), 110.3 (d, ³J_{C-F}=10.1 Hz), 101.2 (d, ³J_{C-F}=3.8 Hz), 56.7.

¹³C {¹⁹F} NMR (126 MHz, CDCl₃): δ 151.9, 145.4, 133.9, 117.0, 110.3, 101.2, 56.7.

¹⁹F {¹H}NMR (376 MHz, CDCl₃): δ −143.1.

HRMS-ESI (m/z): [M+H]⁺ calculated for [C7H₈³⁵ClFNO]⁺:176.0273, found: 176.0271.

m.p.: 49.2-51.5° C. (mean of n=3 replicates)

### Synthesis of 4-chloro-6-methoxy-7-(2-((4-methylpiperazin-1-yl)oxy)ethoxy)quinolone-3-carbonitrile (13)

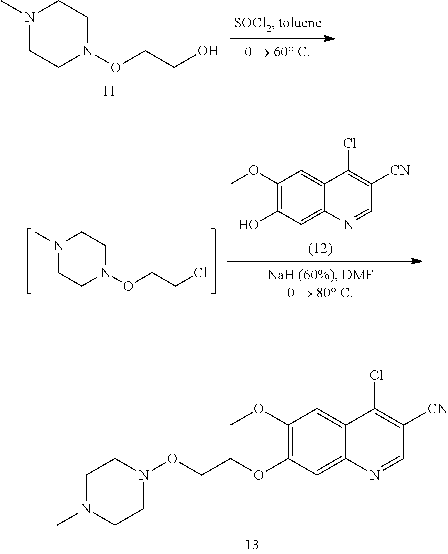

To a stirred solution of 11 (1.15 g, 7.18 mmol, 1.0 eq.) in anhydrous toluene (30 mL) at 0° C. was added SOCl₂ (1.05 mL, 14.36 mmol, 2.5 eq.) dropwise. The solution was stirred for 5 min, after which the mixture was brought to 60° C. and stirred for 3 h. After such time, the solvent was removed in-vacuo and the residue obtained was dissolved in EtOAc (75 mL) and washed with saturated aq. K₂CO₃ soln. (2×, 75 mL). The organic layers were combined, dried over Na₂SO₄, filtered and concentrated in-vacuo. The resulting alkyl chloride was used directly in the next step without further purification.

To a stirred solution of 4-chloro-7-hydroxy-6-methoxyquinoline-3-carbonitrile (12) (2.53 g, 10.77 mmol, 1.5 eq.) in anhydrous DMF (30 mL) at 0° C. was added NaH (60% dispersion in mineral oil) (429 mg, 10.77 mmol, 1.5 eq.) and the solution was stirred for 30 min gradually warming to r.t. After such time, the crude alkyl chloride obtained in the previous step was added dropwise as a solution in anhydrous DMF (5 mL) at r.t. After stirring for 5 min, the solution was brought to 80° C. and stirred for 6 h. After such time, the solvent was removed in-vacuo and the resulting residue co-concentrated with toluene (3×, 50 mL). The residue obtained was dissolved in EtOAc (75 mL) and washed successively with saturated aq. K₂CO₃ soln. (2×, 75 mL) and aq. brine (2×, 75 mL). The organic layer was dried over Na₂SO₄, filtered and concentrated in-vacuo. The residue obtained was purified by flash column chromatography (eluent: 90:5:5 EtOAc:MeOH:Et₃N) to afford the title compound (13) (950 mg, 2.53 mmol, 35% over 2 steps).

Physical State: Yellow powder.

TLC R_{f}=0.50 (90:5:5 EtOAc:MeOH:Et₃N; CAM, UV)

¹H NMR (500 MHz, CDCl₃): δ 7.45 (s, 1H), 7.40 (s, 1H), 4.37 (t, J=5.0 Hz, 2H), 4.16 (t, J=5.0 Hz, 2H), 4.05 (s, 3H), 3.21 (br d, J=10.1 Hz, 2H), 2.78-2.76 (m, 2H), 2.27-2.25 (m, 5H).

¹³C NMR (126 MHz, CDCl₃): δ 155.0, 152.5, 148.3, 147.2, 143.9, 121.2, 115.6, 109.4, 105.6, 102.2, 69.3, 67.7, 56.5, 55.5, 54.1, 45.6.

HRMS-ESI (m/z): [M+H]⁺ calculated for [C₁₈H₂₂N₄O₃³⁵Cl]⁺: 377.1375, found 377.1371.

m.p: 139.1-141.1° C. (mean of n=3 replicates)

### Synthesis of 4-((2,4-dichloro-5-methoxyphenyl)amino)-6-methoxy-7-(2-((4-methylpiperazin-1-yl)oxy)ethoxy)quinoline-3-carbonitrile (7)

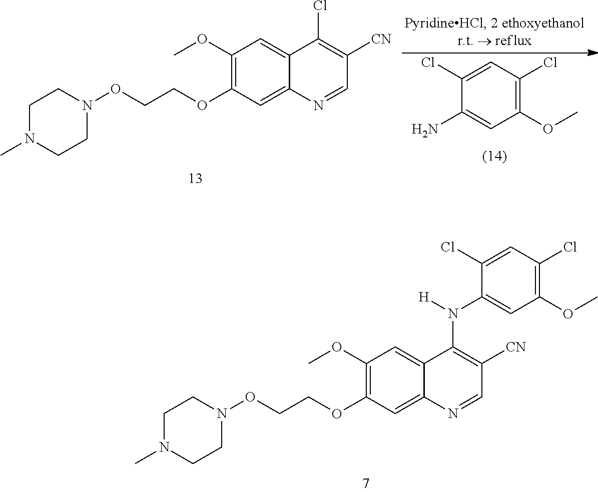

To a stirred solution of 13 (925 mg, 2.46 mmol, 1.0 eq.) in 2-ethoxyethanol (12.5 mL) at r.t. was added pyridine-HCl (569 mg, 4.92 mmol, 2.0 eq.) followed by 2,4-dichloro-5-methoxyaniline (14) (709 mg, 3.69 mmol, 1.5 eq.). The solution was then brought to reflux (135° C.) and stirred for 10 h. After such time, the solvent was removed in-vacuo and the resulting residue co-concentrated with toluene (3×, 30 mL) to remove residual 2-ethoxyethanol. After such time, the residue obtained was purified by flash column chromatography on silica (eluent: 90:5:5 EtOAc:MeOH:Et₃N to 85:10:5 EtOAc:MeOH:Et₃N) to afford the title compound (7) (374 mg, 0.704 mmol, 29%).

Physical State: Light yellow solid.

TLC R_{f}=0.20 (90:5:5 EtOAc:MeOH:Et₃N; CAM, UV)

¹H NMR (500 MHz, CDCl₃): δ 8.71 (s, 1H), 7.49 (s, 1H), 7.44 (s, 1H), 6.90 (s, 1H), 6.73 (s, 1H), 6.46 (s, 1H), 4.36 (t, J=5.0 Hz, 2H), 4.15 (t, J=5.0 Hz, 2H), 3.77 (s, 3H), 3.67 (s, 3H), 3.23-3.21 (m, 2H), 2.78 (br s, 4H), 2.28-2.24 (m, 5H).

¹³C NMR (126 MHz, CDCl₃): δ 154.4, 154.0, 150.5, 150.0, 147.72, 147.66, 137.0, 130.7, 118.6, 117.5, 116.5, 115.0, 110.1, 105.8, 101.3, 94.2, 69.4, 67.5, 56.7, 56.2, 55.4, 53.9, 45.5.

HRMS-ESI (m/z): [M+H]⁺ calculated for [C₂₅H₂₈O₄N₅³⁵Cl₂]: 532.1518, found: 532.1505.

m.p: 183.1-184.7° C. (mean of n=3 replicates).

### Synthesis of 4-((4-chloro-2-fluoro-5-methoxyphenyl)amino)-6-methoxy-7-(2-((4-methylpiperazin-1-yl)oxy)ethoxy)quinoline-3-carbonitrile (15)

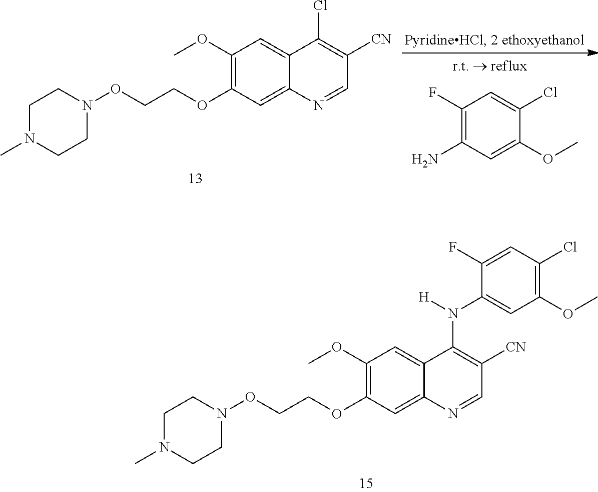

To a stirred solution of 13 (890 mg, 2.37 mmol, 1.0 eq.) in 2-ethoxyethanol (12 mL) at r.t. was added pyridine-HCl (547 mg, 4.74 mmol, 2.0 eq.) followed by 4-chloro-2-fluoro-5-methoxyaniline (658 mg, 3.56 mmol, 1.5 eq.). The solution was then brought to reflux (135° C.) and stirred for 10 h. After such time, the solvent was removed in-vacuo and the resulting residue co-concentrated with toluene (3×, 20 mL) to remove residual 2-ethoxyethanol. After such time, the residue obtained was dissolved in EtOAc (75 mL) and washed with saturated aq. K₂CO₃ (2×, 75 mL). The organic layers were combined and dried over Na₂SO₄, filtered and concentrated in-vacuo. The resulting residue was purified by flash column chromatography on silica (eluent: 85:10:5 EtOAc:MeOH:Et₃N) to afford the title compound (15) (432 mg, 0.837 mmol, 35%).

Physical State: Yellow solid.*

TLC R_{f}=0.30 (90:10:5 EtOAc:MeOH:Et₃N; CAM, UV)

¹H NMR (500 MHz, CDCl₃): δ 8.66 (s, 1H), 7.42 (s, 1H), 7.24 (s, 1H), 6.95 (s, 1H), 6.68 (s, 1H), 6.58 (d, J=7.2 Hz, 1H), 4.34 (t, J=5.0 Hz, 2H), 4.14 (t, J=5.0 Hz, 2H), 3.77 (s, 3H), 3.72 (s, 3H), 3.22-3.20 (m, 2H), 2.85-2.68 (m, 4H), 2.27-2.22 (m, 5H).

¹³C NMR (126 MHz, CDCl₃): δ 153.9, 151.9 (d, ⁴J_{C-F}=1.3 Hz), 150.4, 150.0, 149.1 (d, ¹J_{C-F}=241.9 Hz), 148.1, 147.6, 127.5 (d, ²J_{C-F}=13.9 Hz), 118.8 (d, ³J_{C-F}=10.1 Hz), 118.1 (d, ²J_{C-F}=23.9 Hz), 116.7, 114.5, 110.2, 107.1, 101.1, 93.0, 69.4, 67.5, 57.0, 56.2, 55.5, 54.2, 45.6.

¹³C {¹⁹F} NMR (126 MHz, CDCl₃): δ 153.9, 151.9, 150.4, 150.0, 149.1, 148.1, 147.6, 127.5, 118.8, 118.1, 116.7, 114.5, 110.2, 107.1, 101.1, 93.0, 69.4, 67.5, 57.0, 56.2, 55.5, 45.6.

HRMS-ESI (m/z): [M+H]⁺ calculated for [C₂₅H₂₈O₄N₅³⁵ClF]⁺: 516.1808, found: 516.1805.

m.p: 203.6-204.9° C. (mean of n=3 replicates)

*Note: To ensure no palladium contamination had been carried through from the synthesis of 4-chloro-2-fluoro-5-methoxyaniline by hydrogenation over palladium on carbon, compound 15 was screened by ICP-MS at the University of Georgia's Plasma Chemistry Laboratory in the Center for Applied Isotope Studies, and was found to contain <0.009 μg/g of ¹⁰⁵Pd.

### Results and Discussion

As shown in FIG. 1C, 7 is readily prepared in only 5 steps from readily available starting materials by a route highlighting direct N—O bond forming reaction. In a biochemical inhibition assay, 7 and bosutinib (4) each exhibit potent activity (<1 nM) against imatinib resistant BCR-ABL1 mutants, but as expected³⁶⁻³⁷ show slightly reduced activity against BCR-ABL1^{T315I} which bears the T315I “gatekeeper” mutation that disrupts the ATP binding region drug contact sites through exchange of a threonine residue for an isoleucine (Table 1). Both 7 and bosutinib (4) also strongly inhibit cSRC, an additional target for bosutinib (4), with IC₅₀ values of 2 nM (Table 1).

TABLE 1
Determination of inhibitory activities (IC₅₀) of bosutinib
and compounds 7, 15 against mutant BCR-ABL1 and cSRC.

| | | Compound | Compound |
|---|---|---|---|
| Compound | Bosutinib^{a} | 7 | 15 |
| BCR-ABL1 | | | |
| IC₅₀ (nM)^{b, c} | | | |
| wt | <1 | <1 | 1 |
| H396P | <1 | <1 | <1 |
| M351T | <1 | <1 | 2 |
| Q252H | <1 | <1 | 2 |
| T315I | 44 | 47 | 166 |
| Y253F | <1 | <1 | 1 |
| cSRC | | | |
| IC₅₀ (nM)^{b, c} | | | |
| wt | 2 | 2 | 2 |

^{a}Obtained from MedChemExpress (HY-10158).
^{b}Conducted by Eurofins Cerep SA.
^{c}IC₅₀ (nM) represents mean of n = 2 technical replicates conducted at [ATP] = 10 μM.

The in vitro ADMET properties of 7 were assessed, first measuring lipophilicity where 7 and bosutinib (4) have log D_{7.4} values of 3.5 and 3.1, respectively. Despite the increase in lipophilicity, 7 and bosutinib (4) exhibited similar solubility at pH 7.4 and a comparable unbound plasma protein fraction in both human and rat. Both 7 and bosutinib (4) showed significant degradation in human and rat liver microsomes, while both compounds had comparably enhanced stability in human and rat hepatocytes. Compound 7 also underwent stability determination in mouse and dog hepatocytes where it showed enhanced stability to that seen in rat hepatocytes. Gratifyingly, in a colon-carcinoma (Caco-2) cell permeability assay, which expresses both P-gp and BCRP, 7 exhibited nearly a 4-fold decrease in efflux ratio.³⁸⁻³⁹ This effect was also seen in a Madin-Darby canine kidney (MDCK) MDCKII-MDR1 cell permeability assay, where 7 again exhibited a decreased efflux ratio as compared to bosutinib (4), albeit both compounds were still substrates for P-gp. We also prepared and assessed a second potent BCR-ABL1 inhibitor (15) with single-digit nanomolar biochemical activity, replacing the ortho-chlorine atom present in 7 with an ortho-fluorine atom, but no further improvement in drug efflux was seen. Nevertheless, we were still very encouraged by the increase in apparent permeability and greatly decreased efflux in the Caco-2 cell permeability assay exhibited by 7, and as such, proceeded with measuring the potential of 7 and bosutinib (4) to inhibit the hERG ion channel. Pleasingly, 7 showed over a 3-fold increase in IC₅₀ compared to bosutinib (4) with IC₅₀ values of 1.00 PM and 3.41 μM, respectively, which translated into a reduced maximal % hERG inhibition of 24 for 7 and 52 for bosutinib at 1 μM. Encouraged by the positive attributes of 7, we then conducted an AMES³⁶ fluctuation test across 4 Salmonella strains (TA98, TA100, TA1537 and TA1535) and an in vitro micronucleus⁴⁰ test in Chinese hamster ovaries (CHO) cells, both with and without metabolic activation by S9 (+/−S9), and found that 7 was neither mutagenic nor genotoxic, dispelling concerns regarding the perceived⁴⁰⁻⁴⁴ inherent mutagenicity and genotoxicity of hydroxylamines. In a CYP inhibition assay across all major isoforms (3A4, 1A2, 2C9, 2D6, 2C19) we observed no inhibition for 7 (IC₅₀=>10 μM) except for moderate inhibition of CYP2D6 (IC₅₀=7.6 μM) and CYP2C19 (IC₅₀=1.7 μM). In a follow-up CYP time-dependent-inhibition (TDI) IC₅₀-shift experiment in human liver microsomes, 7 was negative against the highly polymorphous CYP2D6 while showing only moderate time-dependent inhibition potential against CYP2C19.⁴⁵

TABLE 2
In Vitro ADMET Properties of Bosutinib and compounds 7, 15.^{a}

| | | Compound | Compound |
|---|---|---|---|
| Compound | Bosutinib^{b} | 7 | 15 |
| logD_{7.4}^{c} | 3.1 | 3.5 | 3.4 |
| Aq. Sol. (μM)^{d} | 14 | 12 | 43 |
| f_{u, plasma} % (H/R)^{e} | 4.5/7.2 | 5.0/6.5 | 5.6/4.9 |
| LMCIᵢₙₜ (H/R)^{f} | 288/264 | 228/185 | 321/286 |
| t_{1/2} (min) (H/R)^{f} | 4.8/5.3 | 6.1/7.6 | 4.3/4.9 |
| HEPCIᵢₙₜ (H/R)^{g} | 25.3/103.4 | 34.3/178.2 | 40.5/113.1 |
| t_{1/2} (min) (H/R)^{g} | 54.9/13.4 | 40.4/7.8 | 34.2/12.3 |
| Caco-2 Pₐₚₚ | 0.9:10.9 | 1.1:4.0 | 1.9:12.1 |
| (a − b:b − a) (10⁻⁶ | | | |
| cm/s)^{h} | | | |
| Caco-2 ER^{h} | 12.1 | 3.6 | 6.4 |
| MDCKII-MDR1 | 0.4:11.3 | 0.5:10.3 | 0.7:19.3 |
| Pₐₚₚ | | | |
| (a − b:b − a) (10⁻⁶ | | | |
| cm/s)ⁱ | | | |
| MDCKII-MDR1 | 28.3 | 20.6 | 26.5 |
| ERⁱ | | | |
| hERG (IC₅₀) | 1.00 | 3.41 | n.d. |
| (μM)^{j} | | | |
| % hERG | 52/93 | 24/83 | n.d. |
| Inhibition | | | |
| (1 μM/10 μM)^{j} | | | |
| AMES^{k} | n.d. | Negative | n.d. |
| Micronucleus^{l} | n.d. | Negative | n.d. |

^{a}Assayed by Pharmaron Inc. and values presented represent the mean of n = 2 technical replicates, unless otherwise specified.
^{b}Obtained from MedChemExpress (HY-10158).
^{c}logD_{7.4} determined by the shake-flask method, n = 1.
^{d}Solubility (μM) in aqueous phosphate buffer at pH 7.4, n = 1.
^{e}Fraction of unbound drug in human and rat plasma was obtained with test concentration of 5 μM by equilibrium dialysis.
^{f}Rate of metabolism (μL/min/mg) in human and rat liver microsomes.
^{g}Rate of metabolism (μL/min/10⁶ cells) in human and rat hepatocytes.
^{h}Parent compound (5 μM) was incubated for 2 h at 37° C. ER is the efflux ratio and is calculated by Pₐₚₚ (b − a/a − b).
ⁱParent compound (1 μM) was incubated for 2 h at 37° C. ER is the efflux ratio and is calculated by Pₐₚₚ (b − a/a − b).
^{j}Obtained by manual patch-clamp system. 5 doses (30, 10, 3.33, 1.11 and 0.37 μM) were run in triplicate, n = 3, for IC₅₀ determinations.
^{k}AMES fluctuation test performed by Eurofins Panlabs with four common strains (TA98, TA100, TA1537 and TA1535) with and without metabolic (+/− S9) at testing concentrations of 5, 10, 50 and 100 μM. Negative result valid up to 10 μM, after which bacterial cytotoxicity was observed.
^{l}In vitro micronucleus test performed by Eurofins Panlabs with and without metabolic activation (+/− S9) at testing concentrations of 0.2, 0.5, 2, 5, 20, 50, 200, 500 μM. Negative result valid up to 20 μM +S9 and 5 μM −S9, after which cytotoxicity was observed.
Non-standard abbreviations: H, human; R, rat; n.d., not determined.

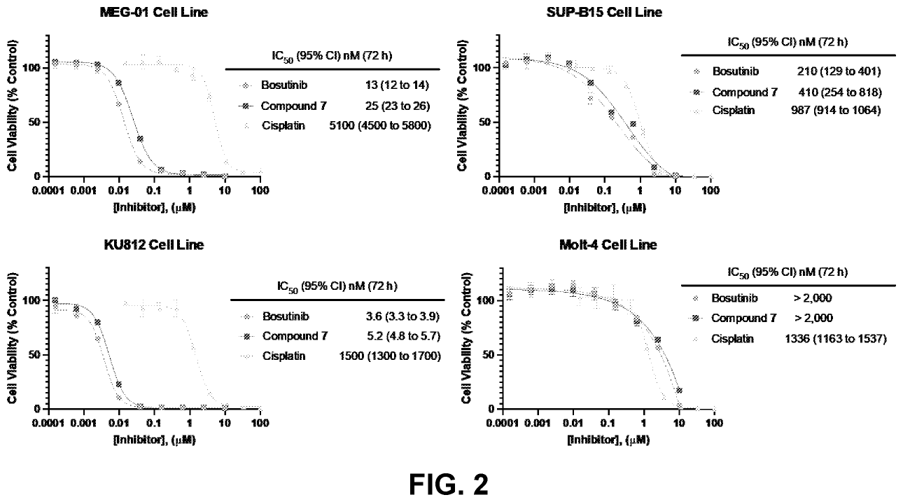

The anti-cancer activity of 7 in four patient-derived leukemia cell lines with bosutinib and cisplatin run as the positive controls in all experiments was performed (FIG. 2). The activity of 7 in Ph+ CML cell lines, KU812 and MEG-01 was strong, which harbor the most common p210 BCR-ABL1 isoform, with IC₅₀ values in the single digit (5.2 nM) to low double digit (25 nM) nanomolar range. In the Ph+ acute lymphoblastic leukemia (ALL) cell line SUP-B15, which bears a shorter p190 isoform of oncogenic BCR-ABL1, 7 showed reduced activity with an IC₅₀ of 410 nM. Finally, to confirm that the antiproliferative activity is due to specific targeting of BCR-ABL1, 7 was assessed in the leukemia cell line MOLT-4, which is negative for BCR-ABL1 and the compound exhibited a markedly decreased IC₅₀ value of >2 μM, as did the parent bosutinib (4).

To qualify 7 as a potential candidate for further evaluation, the pharmacokinetic properties of 7 was assessed after administration into healthy CD1-mice. Importantly, no adverse effects or signs of toxicity were observed for the compound at the tested doses, in stark contrast to the common belief that hydroxylamines are inherently toxic.⁴²⁻⁴⁴ At 5 mg/kg intravenous (IV) and 50 mg/kg oral (PO) dosing, 7 exhibited high oral bioavailability (F=71%), excellent exposure (AUC_{inf}=11,518 h·nM), an acceptable half-life (t_{1/2}=2.18 h) and high maximal plasma concentration (C_{Max}=1,560 nM). Overall, these experiments suggest that 7 is a potent inhibitor of BCR-ABL1 with good oral bioavailability, reduced hERG liability and reduced active drug efflux in Caco-2 cells, all of which were achieved through an insignificant 2 amu hike in molecular weight by incorporation of a “structural alert”.

TABLE 3
Pharmacokinetic profile of compound 7 in male CD1 mice.^{a}

| Dose | Parameter | Value |
|---|---|---|
| 5 mg/kg IV | CL | 52.5 ± 8.6 |
| | (mL/min/kg)^{b} | 2.18 ± 0.24 |
| | t_{1/2} (h)^{c} | 6.39 ± 1.14 |
| | Vₛₛ (L/kg)^{d} | |
| 50 mg/kg | C_{Max} (nM)^{e} | 1,560 ± 701 |
| PO | T_{Max} (h)^{f} | 1.17 ± 0.76 |
| | AUC_{inf} (h · nM)^{g} | 11,519 ± 1,756 |
| | t_{1/2} (hr)^{h} | 2.47 ± 0.03 |
| | F(%)ⁱ | 71.1 ± 10.8 |

^{a}In vivo pharmacokinetics (PK) were performed by Pharmaron Inc. in healthy male CD1 mice via an intravenous infusion route (DMSO: 10% captisol in saline = 1:99) and oral gavage route (0.5% CMC, 2.0% Tween 80, 0.06% acetic acid in H₂O.) PK parameters are presented as mean ± SD, n = 3 mice, per route.
^{b}Clearance obtained from intravenous infusion.
^{c}Mean elimination half-life obtained from intravenous infusion.
^{d}Volumen of distribution at steady state.
^{e}Peak plasme concentration.
^{f}Time to reach peak plasma concentration.
^{g}Area under concentration time curve from 0 to ∞.
^{h}Mean elimination half-life obtained from oral gavage.
ⁱBioavailability (%) calculated with AUC_{inf} and nominal dose.

### CONCLUSIONS

In summary, 7 is an orally bioavailable, potent inhibitor of the BCR-ABL1 kinase with excellent antiproliferative activity in Ph+ CML cell lines KU812 and MEG-01. Considering the improved in vitro profile of 7 compared to bosutinib (4), especially decreased drug efflux and reduced affinity for the hERG channel, 7 has potential as an improved therapeutic for the treatment of p210 BCR-ABL1+ CML, which is expressed in about 95% of CML patients.⁴⁶⁻⁴⁷

### Example 2

### Chemistry Experimental Section

### SYNTHETIC PROCEDURES

### Synthesis of 4-Chloro-2-fluoro-5-methoxyaniline (22)

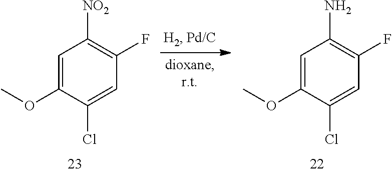

To a stirred solution of 23 (2.5 g, 12.16 mmol, 1.0 eq.) in dioxane at r.t. was added Pd/C (10% by wt) (250 mg), and the solution was evacuated of air and purged 3× with H₂ (g) (1 atm, balloon), after which H₂ (g) (1 atm) was allowed to flow and the reaction was stirred for 20 h at r.t. After such time, the mixture was filtered over Celite® and the solid washed with EtOAc (50 mL). The resulting filtrate was concentrated and the residue obtained was purified by flash column chromatography on silica (eluent: 25:75 EtOAc:hexanes) to afford the title compound 22 (1.82 g, 10.40 mmol, 85%).

Physical State: Light purple solid.

TLC R_{f}=0.10 (25:75 EtOAc:hexanes; UV, CAM).

¹H NMR (500 MHz, CDCl₃): δ 7.01 (d, J=10.3 Hz, 1H), 6.35 (d, J=8.0 Hz, 1H), 3.80 (s, 3H), 3.73 (br s, 2H).

¹³C NMR (126 MHz, CDCl₃): δ 151.9 (d, ⁴J_{C-F}=2.5 Hz), 145.4 (d, ¹J_{C-F}=235.6 Hz), 133.9 (d, ²J_{C-F}=13.9 Hz), 117.0 (d, ²J_{C-F}=23.9 Hz), 110.3 (d, ³J_{C-F}=10.1 Hz), 101.2 (d, ³J_{C-F}=3.8 Hz), 56.7.

¹³C {¹⁹F} NMR (126 MHz, CDCl₃): δ 151.9, 145.4, 133.9, 117.0, 110.3, 101.2, 56.7.

¹⁹F {¹H} NMR (470 MHz, CDCl₃): δ −143.1.

HRMS-ESI (m/z): [M+H]⁺ calculated for [C7H₈NO³⁵ClF]⁺: 176.0273, found: 176.0271.

### Synthesis of 3-Chloro-6-methoxypyridin-2-amine (24) and 5-chloro-6-methoxypyridin-2-amine (26)

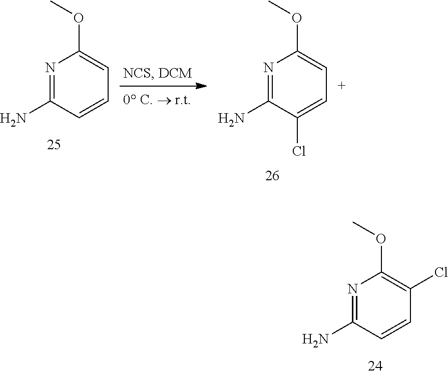

To a stirred solution of 25 (10 g, 80.55 mmol, 1.0 eq.) in DCM at 0° C. was added N-chlorosuccinimide (11.83 g, 88.61 mmol, 1.1 eq.) portion-wise over 10 min. After full addition, the reaction was stirred for 1 h at 0° C., then warmed to r.t. and stirred for an additional 1 h. After such time, NaHCO₃ (200 mL) was added and the layers separated. The organic layer was washed with NaHCO₃ (2×, 200 mL), dried over Na₂SO₄, filtered and concentrated in-vacuo. The residue obtained was purified by flash column chromatography on silica (eluent: 20:80 EtOAc:hexanes) to afford the title compounds as a separable mixture (9.39 g, 59.4 mmol, 74% overall yield; 64% of 3-chloro-6-methoxypyridin-2-amine (26) and 10% of 5-chloro-6-methoxypyridin-2-amine (24)). Spectral data are in accord with the literature.³² 1D-NOESY studies were carried out to support regiochemical assignments. Additionally, 24 was converted to the corresponding benzamide (27) to prove the regiochemistry of chlorination.

### Characterization Data for 3-chloro-6-methoxypyridin-2-amine (26)

Physical state: Brown oil.

TLC R_{f}=0.50 (20:80 EtOAc:hexanes; CAM, UV).

¹H NMR (500 MHz, CDCl₃): δ 7.34 (d, J=8.4 Hz, 1H), 6.07 (d, J=8.4 Hz, 1H), 4.72 (br s, 2H), 3.82 (s, 3H).

¹³C NMR (126 MHz, CDCl₃): δ 162.2, 152.8, 139.5, 105.3, 100.2, 53.8.

### Characterization data for 5-chloro-6-methoxypyridin-2-amine (24)

Physical state: Thick purple oil.

TLC R_{f}=0.40 (20:80 EtOAc:hexanes; Cam, UV).

¹H NMR (500 MHz, CDCl₃): δ 7.33 (d, J=8.2 Hz, 1H), 6.01 (d, J=8.2 Hz, 1H), 4.32 (br s, 2H), 3.92 (s, 3H).

¹³C NMR (126 MHz, CDCl₃): δ 158.3, 155.5, 139.9, 105.4, 100.6, 54.0.

HRMS-ESI (m/z): [M+H]⁺ calculated for [C6HaClN₂O]⁺: 159.0319, found: 159.0315.

### Synthesis of N-5-(Chloro-6-methoxypyridin-2-yl)benzamide (27)

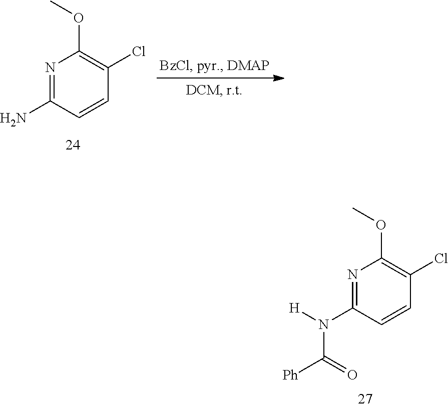

To a stirred solution of 24 (320 mg, 2.77 mmol, 1.0 eq.) in anhydrous DCM (14 mL) was added DMAP (33.8 mg, 0.277 mmol, 0.1 eq.) followed by pyridine (445 μL, 5.53 mmol, 2.0 eq.) and BzCl (322 μL, 2.77 mmol, 1.0 eq.). The reaction was stirred for 12 h at r.t. After such time, the mixture was quenched by addition of NaHCO₃ (25 mL) and the layers separated. The organic layer was further washed with NaHCO₃ (2×, 25 mL), dried over Na₂SO₄, filtered and concentrated in-vacuo. The residue obtained was purified by flash column chromatography on silica (eluent: 20:80 EtOAc:hexanes) to afford the title compound 27 (526 mg, 1.98 mmol, 71%).

Physical state: Light purple solid.

TLC R_{f}=0.50 (20:80 EtOAc:hexanes; CAM, UV).

¹H NMR (500 MHz, CDCl₃): δ 8.31 (s, 1H), 7.92-7.90 (m, 3H), 7.66 (d, J=8.4 Hz, 1H), 7.58 (m, 1H), 7.59-7.49 (m, 2H), 3.97 (s, 3H).

¹³C NMR (126 MHz, CDCl₃): δ 165.5, 158.0, 147.4, 140.5, 134.3, 132.5, 129.0, 127.3, 112.5, 106.7, 54.5.

HRMS-ESI (m/z): [M+H]⁺ calculated for [C₁₃H₁₂O₂N₂³⁵Cl]⁺: 263.0582, found: 263.0574.

The so obtained light purple solid was crystallized from hot EtOAc to obtain colorless crystals suitable for X-ray crystallographic analysis. Crystallographic data has been deposited with the CCDC as entry 2334377.

Crystal m.p. 122.7-124.4° C. (mean of n=3 determinations).

### Synthesis of 4-((3-Chloro-2-flurophenyl)amino)-6-methoxy-7-(2-((4-methylpiperazin-1-yl)oxy)ethoxy)quinoline-3-carbonitrile (16)

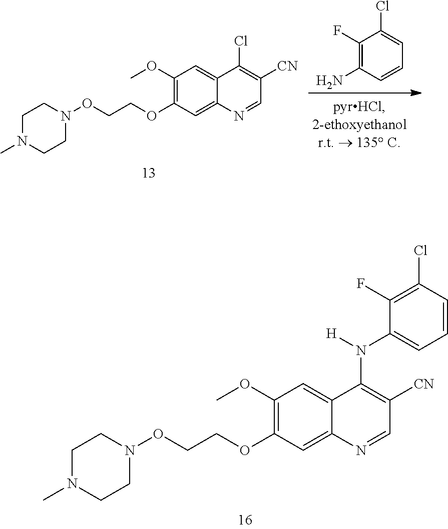 [SMILES: COc1cc2c(Cl)c(C#N)cnc2cc1OCCON1CCN(C)CC1 ; Nc1cccc(Cl)c1F ; COc1cc2c(Nc3cccc(Cl)c3F)c(C#N)cnc2cc1OCCON1CCN(C)CC1]

To a stirred solution of 13 (1.05 g, 2.79 mmol, 1.0 eq.) in 2-ethoxyethanol (15 mL) at r.t. was added pyridine-HCl (645 mg, 5.58 mmol, 2.0 eq.) followed by 3-chloro-2-fluoroaniline (461 μL, 4.19 mmol, 1.5 eq.). The solution was then brought to reflux (135° C.) and stirred for 10 h. After such time, the solvent was removed in-vacuo and the resulting residue co-concentrated with toluene (3×, 25 mL) to remove remaining 2-ethoxyethanol. The residue obtained was dissolved in EtOAc (75 mL) and washed with saturated aq. K₂CO₃ (2×, 75 mL) and the organic layers combined, dried over Na₂SO₄, filtered and concentrated in-vacuo. The resulting residue was purified by flash column chromatography on silica (eluent: 90:5:5 EtOAc:MeOH:Et₃N) to afford the title compound (16) (235 mg, 0.484 mmol, 17%).

Physical state: Light yellow solid.

TLC R_{f}=0.30 (90:5:5 EtOAc:MeOH:Et₃N; CAM, UV).

¹H NMR (500 MHz, CDCl₃): δ 8.62 (s, 1H), 7.38 (s, 1H), 7.15 (t, J=7.4 Hz, 1H), 7.06 (s, 1H), 7.01-6.97 (m, 2H), 6.88 (t, J=7.7 Hz, 1H), 4.31 (t, J=4.9 Hz, 2H), 4.11 (t, J=4.9 Hz, 2H), 3.73 (s, 3H), 3.24-3.18 (m, 2H), 2.77-2.75 (m, 4H), 2.55-2.22 (m, 5H).

¹³C NMR (126 MHz, CDCl₃)*: δ 153.9, 151.2 (d, ¹J_{C-F}=249.5 Hz), 150.4, 149.9, 148.2, 147.4, 130.3 (d, ²J_{C-F}=11.3 Hz), 126.3, 124.4 (d, ³J_{C-F}=5.0 Hz), 122.2 (d, ²J_{C-F}=16.4 Hz), 121.2, 116.6, 114.9, 110.0, 101.4, 93.3, 69.4, 67.4, 56.1, 55.3, 54.0, 45.5.

¹³C {¹⁹F} NMR (126 MHz, CDCl₃): δ 153.9, 151.2, 150.4, 149.9, 148.2, 147.4, 130.3, 126.3, 124.4, 122.2, 121.2, 116.6, 114.9, 110.0, 101.4, 93.4, 69.4, 67.4, 56.1, 55.3, 54.0, 45.5.

¹⁹F {¹H}NMR (470 MHz, CDCl₃): δ −127.6.

HRMS-ESI (m/z): [M+H]⁺ calculated for [C₂₄H₂₆O₃N₅³⁵ClF]⁺: 486.1703, found: 486.1688.

### Synthesis of 4-((2-Chloro-3-methoxyphenyl)amino)-6-methoxy-7-(2-((4-methylpiperazin-1-yl)oxy)ethoxy)quinoline-3-carbonitrile (17)

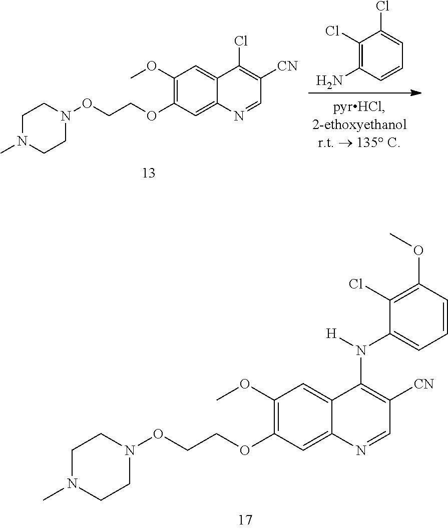 [SMILES: COc1cc2c(Cl)c(C#N)cnc2cc1OCCON1CCN(C)CC1 ; Nc1cccc(Cl)c1Cl ; COc1cc2c(Nc3cccc(OC)c3Cl)c(C#N)cnc2cc1OCCON1CCN(C)CC1]

To a stirred solution of 13 (760 mg, 2.02 mmol, 1.0 eq.) in 2-ethoxyethanol (10 mL) at r.t. was added pyridine-HCl (467 mg, 4.04 mmol, 2.0 eq.) followed by 2-chloro-3-methoxyaniline (478 mg, 3.03 mmol, 1.5 eq.). The solution was then brought to reflux (135° C.) and stirred for 10 h. After such time, the solvent was removed in-vacuo and the resulting residue co-concentrated with toluene (3×, 50 mL) to remove remaining 2-ethoxyethanol. The residue obtained was dissolved in EtOAc (75 mL) and washed with saturated aq. K₂CO₃ (2×, 75 mL) and the organic layers combined, dried over Na₂SO₄, filtered and concentrated in-vacuo. The resulting residue was purified by flash column chromatography on silica (eluent: 85:10:5 EtOAc:MeOH:Et₃N) to afford the title compound (17) (399 mg, 0.803 mmol, 40%).

Physical state: Orange solid.

TLC R_{f}=0.50 (90:5:5 EtOAc:MeOH:Et₃N; CAM, UV).

¹H NMR (500 MHz, CDCl₃): δ 8.68 (s, 1H), 7.41 (s, 1H), 7.11 (t, J=8.3 Hz, 1H), 6.91 (s, 1H), 6.83 (s, 1H), 6.72 (d, J=8.3 Hz, 1H), 6.51 (d, J=8.1 Hz, 1H), 4.34 (t, J=5.0 Hz, 2H), 4.13 (t, J=5.0 Hz, 2H), 3.94 (s, 3H), 3.70 (s, 3H), 3.22-3.20 (m, 2H), 2.77-2.75 (m, 4H), 2.26-2.22 (m, 5H).

¹³C NMR (126 MHz, CDCl₃)*: δ 156.2, 153.9, 150.2, 149.8, 148.3, 147.7, 139.3, 127.2, 116.6, 115.4, 114.2, 113.5, 110.0, 107.3, 102.0, 94.8, 69.4, 67.4, 56.6, 56.1, 55.5, 54.2, 45.6.

HRMS-ESI (m/z): [M+H]⁺ calculated for [C₂₅H₂₉O₄N₅³⁵Cl]⁺: 498.1903, found: 498.1891.

### Synthesis of 4-((2-Fluoro-3-methoxyphenyl)amino)-6-methoxy-7-(2-((4-methylpiperazin-1-yl)oxy)ethoxy)quinoline-3-carbonitrile (18)

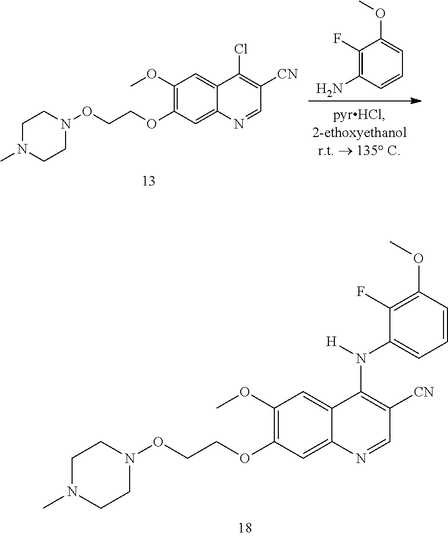

To a stirred solution of 13 (400 mg, 1.06 mmol, 1.0 eq.) in 2-ethoxyethanol (6 mL) at r.t. was added pyridine-HCl (244 mg, 2.12 mmol, 2.0 eq.) followed by 2-fluoro-3-methoxyaniline (226 mg, 1.60 mmol, 1.5 eq.). The solution was then brought to reflux (135° C.) and stirred for 10 h. After such time, the solvent was removed in-vacuo and the resulting residue co-concentrated with toluene (3×, 50 mL) to remove remaining 2-ethoxyethanol. The residue obtained was dissolved in EtOAc (75 mL) and washed with saturated aq. K₂CO₃ (2×, 75 mL) and the organic layers combined, dried over Na₂SO₄, filtered and concentrated in-vacuo. The resulting residue was purified by flash column chromatography on silica (eluent: 90:5:5 EtOAc:MeOH:Et₃N) to afford the title compound (18) (245 mg, 0.509 mmol, 48%).

Physical state: Light orange solid.

TLC R_{f}=0.40 (90:5:5 EtOAc:MeOH:Et₃N; CAM, UV).

¹H NMR (500 MHz, CDCl₃): δ 8.64 (s, 1H), 7.40 (s, 1H), 7.00 (t, J=7.4 Hz, 1H), 6.96 (s, 1H), 6.79 (t, J=8.0 Hz, 1H), 6.69 (s, 1H), 6.60 (t, J=7.5 Hz, 1H), 4.34 (t, J=5.0 Hz, 2H), 4.14 (t, J=5.1 Hz, 2H), 3.92 (s, 3H), 3.70 (s, 3H), 3.27-3.16 (m, 2H), 2.88-2.62 (m, 4H), 2.27-2.23 (m, 5H).

¹³C NMR (126 MHz, CDCl₃)*: δ 153.7, 150.1, 149.9, 148.75 (d, ²J_{C-F}=10.8 Hz), 148.73, 147.6, 145.6 (d, ¹J_{C-F}=245.7 Hz), 129.8 (d, ²J_{C-F}=8.8 Hz), 123.8 (d, ³J_{C-F}=5.0 Hz), 116.8, 115.0, 114.6, 110.1, 109.7, 101.7, 93.2, 69.4, 67.4, 56.6, 56.0, 55.5, 54.2, 45.6.

¹³C {¹⁹F}NMR (126 MHz, CDCl₃): δ 153.7, 150.1, 149.9, 148.8, 148.7, 147.6, 145.6, 129.8, 123.8, 116.7, 115.0, 114.6, 110.1, 109.7, 101.8, 93.2, 69.4, 67.4, 56.6, 56.0, 55.5, 54.2, 45.6.

¹⁹F {¹H}NMR (470 MHz, CDCl₃): −148.2.

HRMS-ESI (m/z): [M+H]⁺ calculated for [C₂₅H₂₉O₄N₅³⁵Cl]⁺:482.2198, found: 482.2196.

### Synthesis of 4-((4-Chloro-3-methoxyphenyl)amino)-6-methoxy-7-(2-((4-methylpiperazin-1-yl)ethoxy)quinoline-3-carbonitrile (19)

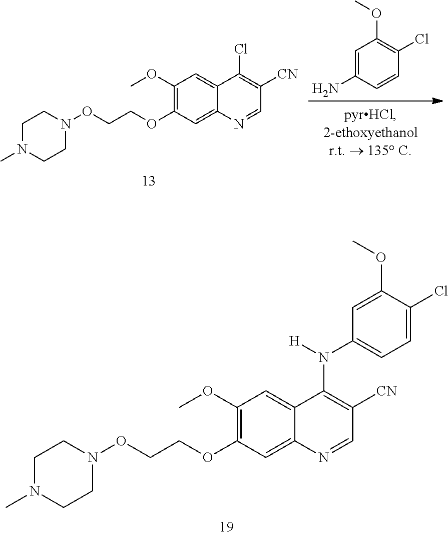

To a stirred solution of 13 (900 mg, 2.39 mmol, 1.0 eq.) in 2-ethoxyethanol (12 mL) at r.t. was added pyridine-HCl (552 mg, 4.78 mmol, 2.0 eq.) followed by 4-chloro-3-methoxyaniline (566 mg, 3.59 mmol, 1.5 eq.). The solution was then brought to reflux (135° C.) and stirred for 10 h. After such time, the solvent was removed in-vacuo and the resulting residue co-concentrated with toluene (3×, 20 mL) to remove remaining 2-ethoxyethanol. The residue obtained was dissolved in EtOAc (100 mL) and washed with saturated aq. K₂CO₃ (2×, 75 mL) and the organic layers combined, dried over Na₂SO₄, filtered and concentrated in-vacuo. The resulting residue was purified by flash column chromatography on silica (eluent: 90:5:5 EtOAc:MeOH:Et₃N) to afford the title compound (19) (240 mg, 0.483 mmol, 20%).

Physical state: Tan solid.

TLC R_{f}=0.25 (90:5:5 EtOAc:MeOH:Et₃N; CAM, UV).

¹H NMR (500 MHz, CDCl₃): δ 8.63 (s, 1H), 7.37 (s, 1H), 7.29 (d, J=8.4 Hz, 1H), 6.97 (s, 1H), 6.88 (s, 1H), 6.66 (d, J=2.4 Hz, 1H), 6.58 (dd, J=8.4, 2.4 Hz, 1H), 4.31 (t, J=5.0 Hz, 2H), 4.12 (t, J=4.9 Hz, 2H), 3.80 (s, 3H), 3.63 (s, 3H), 3.22-3.19 (m, 2H), 2.78-2.76 (m, 2H), 2.26 (br s, 5H).

¹³C NMR (126 MHz, CDCl₃)*: δ 155.8, 153.7, 149.8, 148.9, 147.7, 140.7, 130.6, 118.9, 117.0, 114.8, 114.1, 110.0, 106.6, 102.5, 92.8, 69.4, 67.4, 56.4, 56.0, 55.4, 54.0, 45.5.

HRMS-ESI (m/z): [M+H]⁺ calculated for [C₂₅H₂₉O₄N₅³⁵Cl]⁺: 498.1903, found: 498.1906.

### Synthesis of 4-((4-Chloro-2-methoxyphenyl)amino)-6-methoxy-7-(2-((4-methylpiperazin-1-yl)oxy)ethoxy)quinoline-3-carbonitrile (20)

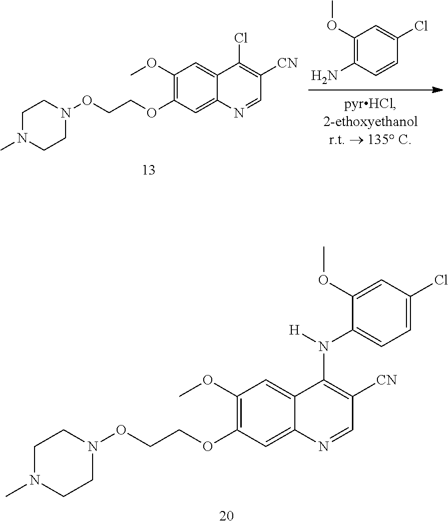

To a stirred solution of 13 (700 mg, 1.86 mmol, 1.0 eq.) in 2-ethoxyethanol (12 mL) at r.t. was added pyridine-HCl (430 mg, 3.72 mmol, 2.0 eq.) followed by 4-chloro-2-methoxyaniline (440 mg, 2.79 mmol, 1.5 eq.). The solution was then brought to reflux (135° C.) and stirred for 14 h. After such time, the solvent was removed in-vacuo and the resulting residue co-concentrated with toluene (3×, 50 mL) to remove remaining 2-ethoxyethanol. The residue obtained was dissolved in EtOAc (100 mL) and washed with saturated aq. K₂CO₃ (2×, 75 mL) and the organic layers combined, dried over Na₂SO₄, filtered and concentrated in-vacuo. The resulting residue was purified by flash column chromatography on silica (eluent: 90:5:5 EtOAc:MeOH:Et₃N) to afford the title compound (20) (153 mg, 0.307 mmol, 17%).

Physical state: as a brown solid.

TLC R_{f}=0.40 (90:5:5 EtOAc:MeOH:Et₃N; CAM, UV).

¹H NMR (500 MHz, CDCl₃): δ 8.62 (s, 1H), 7.39 (s, 1H), 6.98-6.92 (m, 2H), 6.87-6.85 (m, 1H), 6.81 (d, J=8.1 Hz, 2H), 4.33 (t, J=5.0 Hz, 2H), 4.14 (t, J=5.0 Hz, 2H), 3.90 (s, 3H), 3.72 (s, 3H), 3.25-3.23 (m, 2H). 2.83-2.81 (m, 4H), 2.31 (s, 5H).

¹³C NMR (126 MHz, CDCl₃)*: δ 153.6, 151.6, 150.0, 149.9, 148.8, 147.5, 130.1, 128.5, 121.6, 120.4, 117.0, 114.6, 112.1, 110.1, 102.1, 93.1, 69.5, 67.4, 56.2, 56.1, 54.8, 45.4.

HRMS-ESI (m/z): [M+H]⁺ calculated for [C₂₅H₂₉O₄N₅³⁵Cl]⁺: 498.1903, found: 498.1890.

### Synthesis of 4-((5-Chloro-6-methoxypyridin-2-yl)amino)-6-methoxy-7-(2-((4-methylpiperazin-1-yl)oxy)ethoxy)quinoline-3-carbonitrile (21)

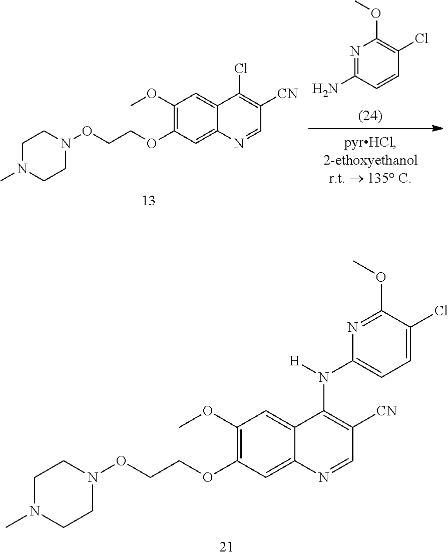

To a stirred solution of 13 (335 mg, 0.89 mmol, 1.0 eq.) in 2-ethoxyethanol (8 mL) at r.t. was added pyridine-HCl (206 mg, 1.78 mmol, 2.0 eq.) followed by 5-chloro-6-methoxypyridin-2-amine (24) (281 mg, 1.78 mmol, 2.0 eq.). The solution was then brought to reflux (135° C.) and stirred for 6 h. After such time, the solvent was removed in-vacuo and the resulting residue co-concentrated with toluene (3×, 50 mL) to remove remaining 2-ethoxyethanol. The residue obtained was dissolved in EtOAc (100 mL) and washed with saturated aq. K₂CO₃ (2×, 75 mL) and the organic layers combined, dried over Na₂SO₄, filtered and concentrated in-vacuo. The resulting residue was purified by flash column chromatography on silica (eluent: 90:5:5 EtOAc:MeOH:Et₃N) to afford the title compound (21) (58 mg, 0.116 mmol, 13%).

Physical state: as a light yellow solid.

TLC R_{f}=0.15 (90:5:5 EtOAc:MeOH:Et₃N; CAM, UV).

¹H NMR (500 MHz, CDCl₃): δ 8.74 (s, 1H), 7.50 (d, J=8.1 Hz, 1H), 7.43 (s, 1H), 7.38 (s, 1H), 7.08 (s, 1H), 6.29 (d, J=8.2 Hz, 1H), 4.34 (t, J=5.0 Hz, 2H), 4.14 (t, J=4.9 Hz, 2H), 3.85 (s, 3H), 3.82 (s, 3H), 3.28-3.19 (m, 2H), 2.84-2.82 (m, 4H), 2.37-2.30 (m, 5H).

¹³C NMR (126 MHz, CDCl₃)*: δ 158.3, 154.1, 150.6, 150.4, 149.4, 147.8, 146.7, 140.0, 117.0, 116.9, 110.5, 109.8, 103.7, 102.1, 97.4, 69.5, 67.5, 56.3, 54.9, 54.4, 53.7, 45.3.

HRMS-ESI (m/z): [M+H]⁺ calculated for [C₂₅H₂₉O₄N₅³⁵Cl]⁺: 499.1855, found: 499.1850.

### Proposed Synthesis of Pyrimidine (29) Via Buchwald Hartwiq Couplinq⁴⁸

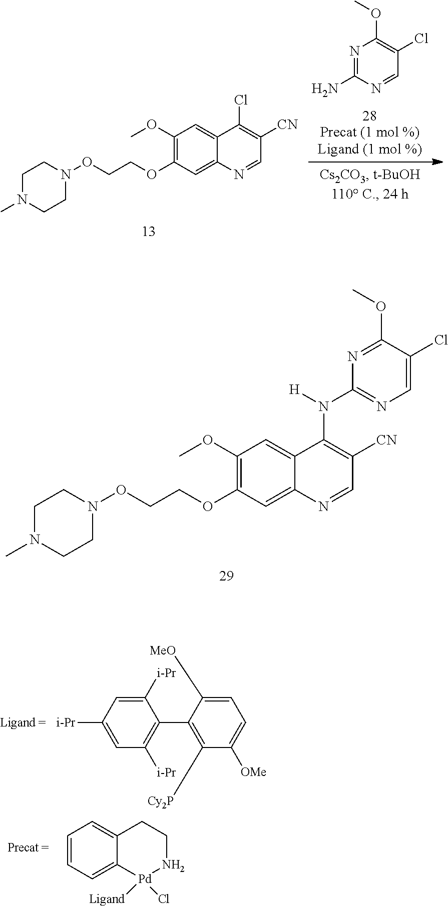

Synthesis of pyrimidine-based BCR-ABL1 inhibitor will be carried out under Buchwald-Hartwig amination conditions as previously described in Reference 48. Pyrimidine (28) can be prepared by chlorination of commercially available 4-methoxy-2-pyridinamine [CAS: 155-90-8] according to the scheme below:

### Synthesis of 5-Chloro-4-methoxypyrimidin-2-amine (28)

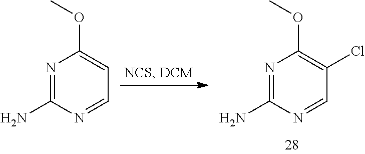

To a stirred solution of 4-methoxy-2-pyridinamine (2 g, 15.98 mmol, 1.0 eq.) in DCM (60 mL) was added N-chlorosuccinimide (NCS) (3.2 g, 23.97 mmol, 1.5 eq.) at r.t. and the solution was stirred for 9 h. After such time, the mixture was diluted with DCM (50 mL) and washed with 1 N NaOH (2×, 100 mL). The combined organic layers were dried over Na₂SO₄, filtered and concentrated in vacuo. The residue obtained was purified by flash column chromatography on silica (eluent: 70:30 EtOAc:hexanes) to afford the title compound (28) (1.86 g, 11.66 mmol, 73% yield).

Physical State: White solid

TLC R_{f}=0.80 (70:30 EtOAc:hexanes: UV, CAM)

¹H NMR (500 MHz, CDCl₃): δ 8.00 (s, 1H), 5.03 (br s, 2H), 3.96 (s, 3H).

¹³C NMR (126 MHz, CDCl₃): δ 165.2, 161.3, 156.4, 106.5, 54.4.

HRMS-ESI (m/z): [M+H]⁺ calculated for [C₅H₇ON₃³⁵Cl]⁺: 160.0272, found: 160.0267. Biological Materials and Data

### Cell Lines

The K562 cells were obtained from ATCC. K562/Dox cells were prepared by treatment of K562 cells with 60 nM doxorubicin (in DMSO) for one week.⁴⁹ MDCKII-MDR1 cells were obtained from the Netherlands Cancer Institute. Caco-2 cells were obtained from the ATCC for permeability studies and flow cytometry studies. DLD1 and DU4475 cells were obtained from the ATCC. MDCKII-MDR1 cells were seeded at a density of 1.56×10⁶ cells/mL and cultivated for 4-8 days prior to assays. Caco-2 cells were seeded at a density of 6.86×10⁵ cells/mL and cultivated for 14-18 days prior to permeability assays.

### Cell Viability Assay

K562 cells were plated in 96-well plates at 2,000 cells per well and dosed in triplicate (n=3) in a 12-point, 5-fold dilution series with compounds (0.2048 μM to 10 PM; 25 μM and 50 μM doses were also included in the run) in DMSO and incubated for 72 h. After 72 h, cell viability was assayed by CellTiter-Glo Luminescnet Viability Assay (Promega). Dose-response curves were generated and used to calculate the IC₅₀ values which were calculated on GraphPad Prism from the nonlinear regression equation fitted with a sigmoidal dose-response and are presented as mean (95% confidence interval).

### In Vitro ADMET

Lipophilicity, solubility, plasma protein binding, metabolic stability in hepatocytes, permeability studies in Caco-2 cells and MDCKII-MDR1 cells, hERG channel inhibition and CYP inhibition was determined by Pharmaron Inc. using methods described in Example 1.

### Statistical Analyses

Statistical analysis was performed using GraphPad Prism 9.0. Data are presented as the mean±SD or SEM as indicated when n=≥3, or as the geometric mean when n=2. For in vitro ADMET and kinase activity studies, data is presented as mean of n≥2 independent replicates. For in vitro short-term growth delay experiments, IC₅₀ values were determined from the nonlinear regression equation fitted with a sigmoidal dose-response curve and are presented as the mean±SD, n=3 independent replicates and IC₅₀ values are reported beside the dose-response curve and represent the mean (95% confidence interval).

### Results

Provided is the identification of a potent dual cSRC/BCR-ABL1 kinase inhibitor, with a reduced P-gp-mediated efflux ratio relative to bosutinib and potent activity in a Ph+ patient derived cell line (K562) and an MDR-Ph+ patient derived cell line (K562/Dox) overexpressing P-gp. Compared to bosutinib, the optimal compound has an over 300-fold improvement in relative resistance in K562/Dox cells and an approximately 60-fold improvement in activity compared to imatinib, and thus is a potential lead in the treatment of MDR Ph+ CML characterized by P-gp overexpression.

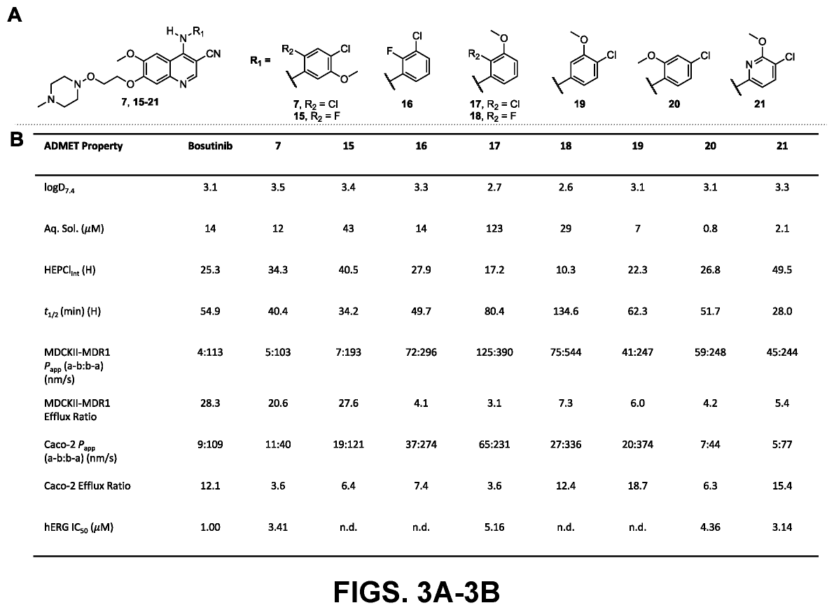

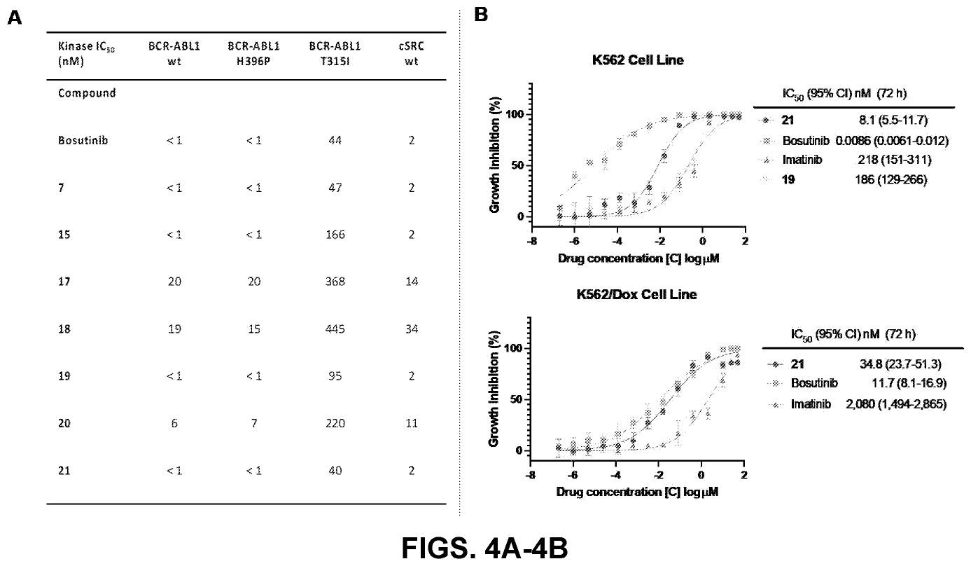

Compound 15 was prepared and characterized by the ortho-chlorine to ortho-fluorine switch from 7 (FIG. 3a). This modification had minimal effect on biochemical inhibition of the relevant mutant kinases (IC₅₀'s<1 nM), but unexpectedly increased the P-gp-mediated efflux ratio (MDCKII-MDR1 efflux ratio=27.6) relative to parent 7 (MDCKII-MDR1 efflux ratio=20.3) (vide infra). Moving forward, we then synthesized a series of 4-anilinoquinoline-3-carbonitrile BCR-ABL1 inhibitors which differ in substitution about the pendant anilino moiety (15-21), and determined their early ADMET parameters and their ability to inhibit mutant BCR-ABL1 and cSRC (vide infra). Interestingly, 17 displayed a 31-fold enhancement in permeability and a greatly reduced efflux ratio in MDCKII-MDR1 cells relative to bosutinib, highlighting the utility of decreasing hydrogen bond acidity to improve P-gp permeability with adjacent electron-withdrawing substituents (FIG. 3). Moreover, 17 exhibited high aqueous solubility at pH 7.4, good stability in human hepatocytes and minimal hERG inhibition (IC₅₀=5.16 μM). With regards to kinase inhibition, however, 17 and 18 displayed comparatively poor activity against mutant BCR-ABL1 kinases with IC₅₀ values ≥15 nM (FIG. 4a). This result is consistent with previous structure activity relationships (SAR) that have established the importance of para-substituents for cSRC and BCR-ABL1 kinase activity (FIG. 4a).⁵⁰⁻⁵¹ Thus, we focused our attention on 19 and 20 which bear the 4-chloro substituent. Fortunately, 19 and 20 exhibited markedly improved permeability and reduced efflux ratios in MDCKII-MDR1 cells and both compounds (19 and 20) displayed good stability in human hepatocytes. Finally, we assayed compound 21, which bears the pyridylamine moiety, and observed excellent activity against both BCR-ABL1 and cSRC kinases, markedly improved permeability and a reduced efflux ratio in MDCKII-MDR1 cells relative to bosutinib.

Taking kinase activity and efflux ratio in MDCKII-MDR1 cells into consideration, we profiled 19 and 21 in a Ph+ patient-derived leukemia cell line, K562 (FIG. 4a-b). Imatinib and 19 both had activity in the low triple-digit nanomolar range (IC₅₀ values of 186 nM and 218 nM), while 21 with an IC₅₀ of 8.1 nM was closer to the picomolar activity of bosutinib, leading us to focus subsequent studies on 21. In additional ADMET profiling, 21 displayed a pH-dependent solubility profile with excellent solubility below pH 6.5, good stability in human and mouse hepatocytes and high human and mouse plasma protein binding (FIG. 3). With regards to potential toxicity, 21 showed only moderate inhibition of the hERG potassium ion channel with an IC₅₀ value of 3.14 μM and a maximal inhibition of approximately 20% at 1 μM (FIG. 3). The cytochrome P450 (CYP) activity of 21 was then assayed across four major isoforms (3A4, 1A2, 2D6, 2C9) resulting in IC₅₀'s>10 μM except for CYP3A⁴ against which 21 had an IC₅₀ of 1.0 μM (see Experimental Section for details). Fortunately, 21 was negative in a follow-up time-dependent inhibition (TDI) IC₅₀-shift experiment in human liver microsomes, thereby dispelling concerns of potential drug-drug interactions (DDI).⁴⁵ We then determined the antiproliferative activity of 21 in patient-derived K562 cells pre-treated with the chemotherapeutic doxorubicin (Dox), which leads to an MDR phenotype characterized by P-gp overexpression (FIG. 4b).⁵²⁻⁵³ Gratifyingly, we observed only a minimally increased IC₅₀ value of 34.8 nM, representing an approximately 4-fold shift for resistance compared to wild-type K562 cells. In contrast, imatinib and bosutinib had IC₅₀ values of 2,080 nM and 11.7 nM, respectively, in the K562/Dox cells representing approximately 10-fold and 1360-fold resistance, respectively. Overall, 21 exhibits a >10-fold improvement in permeability and a markedly reduced efflux ratio in MDCKII-MDR1 cells relative to bosutinib and is a potent inhibitor in imatinib-resistant and bosutinib-resistant patient derived Ph+ leukemia cells that overexpress P-gp.

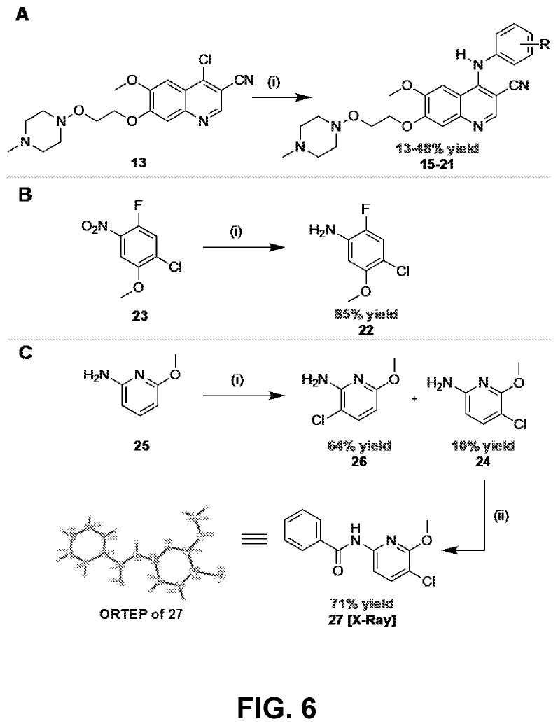

The synthesis of all inhibitors was carried out in an analogous fashion to that previously reported for preparation of 7 in Example 1 and Reference 31 (FIG. 6). To this end, a nucleophilic aromatic substitution (S_{N}Ar) reaction with the substituted anilines under acidic conditions with quinoline (13) gave the desired inhibitors in 13-48% yield (FIG. 6a). The anilines were commercially available except for 22 (4-chloro-2-fluoro-5-methoxyaniline) and 24 (5-chloro-6-methoxypyridin-2-amine). Consequently, aniline 22 was prepared in 85% yield by hydrogenation of the corresponding nitro derivative (23) over palladium on carbon (Pd/C), while aniline 24 was prepared by chlorination of 25 with N-chlorosuccinimide (NCS) to give a separable mixture of regioisomers (26 and 24) in 64 and 10% yields, respectively (FIG. 4b).³² The regiochemistry of the chlorination was confirmed through a series of 1D-NOESY experiments and was proved for 24 after conversion to the corresponding benzamide derivative (27) and single crystal X-ray analysis (FIG. 6c).

In summary, a hydroxylamine-bearing dual cSRC/BCR-ABL1 kinase inhibitor with a reduced P-gp efflux ratio and potent activity in a MDR patient-derived leukemia cell line overexpressing P-gp (K562/Dox), compound 21, has been developed. Compared to bosutinib, 21 has an over 300-fold improvement in relative resistance in K562/Dox cells and an approximately 60-fold improvement in activity compared to imatinib. The results described herein could aid future inhibitor design targeting MDR phenotypes characterized by P-gp overexpression.

### Biochemical Human RTK Kinase Enzymatic Radiometric Assay, KinaseProfiler

TABLE 4
Full biochemical IC₅₀ results for compounds
against BCR-ABL1 and cSRC.

| Kinase | Bosutinib^{b} | 7^{b} | 15 | 17 | 18 | 19 | 20 | 21 |
|---|---|---|---|---|---|---|---|---|
| BCR-ABL1 | | | | | | | | |
| IC₅₀ (nM)^{a} | | | | | | | | |
| Wt | <1 | 1 | 1 | 20 | 19 | <1 | 6 | <1 |
| H396P | <1 | <1 | <1 | 20 | 15 | <1 | 7 | <1 |
| M351T | <1 | <1 | 2 | 31 | 24 | 1 | 13 | <1 |
| Q252H | <1 | <1 | 2 | 37 | 30 | <1 | 10 | <1 |
| T315I | 44 | 47 | 166 | 368 | 445 | 95 | 220 | 40 |
| cSRC IC₅₀ (nM)^{a} | | | | | | | | |
| wt | 2 | 2 | 2 | 14 | 34 | 2 | 11 | 2 |

^{a}Conducted by Eurofins Cerep, SA. IC₅₀ values represent the mean of n = 2 technical replicates conducted at [ATP] = 10 μM. Values <1 nM were below the sensitivity of the test and as such are reported as <1 nM.
^{b}IC₅₀ values as previously reported in J. Hill, R. M. Jones, D. Crich. ACS Med. Chem. Lett. 2023, 14, 1869-1875 and in Example 1.

### Plasma Protein Binding by Equilibrium Dialysis

TABLE 5
Plasma details used in the plasma protein binding assay.

| Item | Supplier |
|---|---|
| Human plasma (mixed gender) | BioIVT |
| Mouse plasma (CD-1/mixed gender) | IPHASE or BiolVT |

TABLE 6
Results for plasma protein binding in human and mouse plasma.

| | | | % Un- | % Re- | % Remaining |
|---|---|---|---|---|---|
| Compound | Species | % Bound | bound | covery | at 6 h |
| Ketoconazole | Human | 99.20 | 0.80 | 101.64 | 94.60 |
| Bosutinib^{a} | Human | 95.47 | 4.52 | 94.01 | 102.03 |
| 7^{a} | Human | 95.05 | 4.95 | 92.41 | 101.74 |
| 15 | Human | 94.42 | 5.58 | 99.90 | 92.70 |
| 17 | Human | 92.63 | 7.37 | 92.54 | 97.83 |
| 18 | Human | 87.03 | 12.97 | 87.76 | 98.29 |
| 20 | Human | 92.27 | 7.73 | 95.83 | 99.42 |
| 21 | Human | 97.52 | 2.48 | 93.86 | 97.78 |
| Ketoconazole | Mouse | 99.39 | 0.61 | 102.46 | 103.53 |
| 17 | Mouse | 88.07 | 11.93 | 92.03 | 96.71 |
| 20 | Mouse | 90.29 | 9.71 | 95.87 | 106.50 |
| 21 | Mouse | 94.31 | 5.69 | 93.05 | 97.78 |

^{a}Values as previously reported in J. Hill, R. M. Jones, D. Crich. ACS Med. Chem. Lett. 2023, 14, 1869-1875 and in Example 1.

### Metabolic Stability in Hepatocytes

TABLE 7
Hepatocyte details used in the hepatocyte stability assay.

| Item | Supplier |
|---|---|
| Human Hepatocytes, Mixed-Gender | BioIVT (Cat No. X008001) |
| Mouse Hepatocytes, Male-Gender | BioIVT (Cat No. M005052) |

TABLE 8
Results for hepatocyte stability data in human hepatocytes.

| | | Remaining Percentages (%) | | | | | |
|---|---|---|---|---|---|---|---|
| | | 0.5 | 15 | 30 | 60 | 90 | 120 |
| Compound | Species | min | min | min | min | min | min |
| Verapamil | Human | 100.00 | 56.11 | 35.65 | 17.71 | 8.83 | 4.83 |
| Bosutinib^{a} | Human | 100.00 | 73.99 | 79.28 | 53.95 | 34.15 | 20.73 |
| 7^{a} | Human | 100.00 | 90.07 | 69.58 | 44.09 | 23.58 | 13.59 |
| 15 | Human | 100.00 | 69.28 | 52.48 | 29.45 | 15.72 | 9.48 |
| 16 | Human | 100.00 | 82.49 | 66.92 | 45.06 | 29.85 | 18.60 |
| 17 | Human | 100.00 | 87.31 | 80.64 | 60.46 | 46.43 | 35.84 |
| 18 | Human | 100.00 | 97.02 | 95.67 | 80.41 | 67.22 | 54.89 |
| 19 | Human | 100.00 | 83.13 | 68.12 | 50.16 | 37.41 | 25.52 |
| 20 | Human | 100.00 | 78.44 | 63.53 | 43.29 | 27.95 | 20.14 |
| 21 | Human | 100.00 | 71.19 | 50.26 | 22.99 | 9.44 | 4.39 |
| Verapamil | Mouse | 100.00 | 16.71 | 4.39 | 1.24 | BLOD | BLOD |
| 20 | Mouse | 100.00 | 55.53 | 28.57 | 13.26 | 6.75 | 2.89 |
| 21 | Mouse | 100.00 | 66.11 | 33.68 | 19.55 | 8.83 | 3.16 |

^{a}Values as previously reported in J. Hill, R. M. Jones, D. Crich. ACS Med. Chem. Lett. 2023, 14, 1869-1875 and in Example 1.
Abbreviations: BLOD, below level of detection.

### MDCKII-MDR1 Permeability

TABLE 9
MDCKII-MDR1 cellular details used in
the MDCKII-MDR1 permeability assay.

| Item | Supplier |
|---|---|
| MDCKII-MDR1 cells | Netherlands Cancer Institute (Amsterdam) |
| HTS Transwell 96 Well | Corning (Cat No. 3391) |

TABLE 10
Results for MDCKII-MDR1 cellular permeability.

| | P_{app(a-b)} | P_{app(b-a)} | | | |
|---|---|---|---|---|---|
| | (10⁻⁶, | (10⁻⁶, | Efflux | Recovery % | Recovery % |
| Compound | cm/s) | cm/s) | Ratio | (AP − BL) | (BL − AP) |
| Metoprolol | 31.60 | 28.05 | 0.89 | 111.64 | 99.15 |
| Digoxin | 0.62 | 13.94 | 22.68 | 82.38 | 93.20 |
| Bosutinib^{a} | 0.38 | 11.34 | 29.85 | 56.02 | 65.52 |
| 7^{a} | 0.52 | 10.28 | 19.78 | 45.83 | 58.85 |
| 15 | 0.73 | 19.30 | 26.45 | 79.78 | 64.87 |
| 16 | 7.16 | 29.64 | 4.15 | 71.59 | 85.59 |
| 17 | 12.48 | 38.99 | 3.22 | 94.96 | 91.60 |
| 18 | 7.53 | 54.36 | 7.26 | 93.06 | 105.61 |
| 19 | 4.08 | 24.71 | 6.05 | 75.42 | 77.51 |
| 20 | 5.90 | 24.77 | 4.20 | 84.45 | 72.29 |
| 21 | 4.53 | 24.42 | 5.39 | 76.05 | 73.24 |

^{a}Values as previously reported in J. Hill, R. M. Jones, D. Crich. ACS Med. Chem. Lett. 2023, 14, 1869-1875.
Abbreviations: Pₐₚₚ, apparent permeability; AP, apical; BL, basolateral.

### Caco-2 Permeability

TABLE 11
Caco-2 cellular details used in the
Caco-2 cellular permeability assay.

| Item | Supplier |
|---|---|
| Caco-2 cells | ATCC (ATCC No. HTB-37) |
| HTS Transwell 96 Well | Corning (Cat No. 3391) |

TABLE 12
Results for Caco-2 cellular permeability.

| | P_{app(a-b)} | P_{app(b-a)} | | | |
|---|---|---|---|---|---|
| | (10⁻⁶, | (10⁻⁶, | Efflux | Recovery % | Recovery % |
| Compound | cm/s) | cm/s) | Ratio | (AP − BL) | (BL − AP) |
| Metoprolol | 18.76 | 19.39 | 1.04 | 95.33 | 95.54 |
| Digoxin | 0.22 | 12.58 | 58.64 | 89.90 | 92.72 |
| Bosutinib^{a} | 0.86 | 10.87 | 12.70 | 52.12 | 70.73 |
| 7^{a} | 1.05 | 4.04 | 3.88 | 39.68 | 54.25 |
| 15 | 1.94 | 12.08 | 6.22 | 61.16 | 72.31 |
| 16 | 3.66 | 27.39 | 7.49 | 60.54 | 80.51 |
| 17 | 6.46 | 23.06 | 3.58 | 76.24 | 87.19 |
| 18 | 2.72 | 33.58 | 12.36 | 84.83 | 92.56 |
| 19 | 1.99 | 37.41 | 18.96 | 65.43 | 92.37 |
| 20 | 0.71 | 4.44 | 6.24 | 72.15 | 78.22 |
| 21 | 0.46 | 7.70 | 16.70 | 72.75 | 68.21 |

^{a}Values as previously reported in J. Hill, R. M. Jones, D. Crich. ACS Med. Chem. Lett. 2023, 14, 1869-1875 and in Example 1.
Abbreviations: Pₐₚₚ, apparent permeability; AP, apical; BL, basolateral.

hERG Safety Evaluation by Manual Patch-Clamp System

TABLE 13
HEK293 cellular details used in hERG safety evaluation.

| Item | Supplier |
|---|---|
| HEK 293 Cell Line | Invitrogen (Cat No. K1236) |
| TrypLE ™ Express | Gibco (Cat No. 12604) |
| Dofetilide | TRC (Cat No. D525700) |

TABLE 14
hERG safety evaluation results.

| Compound | hERG IC₅₀ (μM)^{a} |
|---|---|
| Dofetilide^{b, c} | 0.015 ± 0.0008 |
| Bosutinib^{c} | 1.01 ± 0.4 |
| 7^{c} | 3.41 ± 0.3 |
| 17 | 5.16 ± 1.1 |
| 20^{d} | 4.36 ± 1.4 |
| 21^{d} | 3.14 ± 0.8 |

^{a}IC₅₀ values are presented as the mean ± SEM, n = 3 independent replicates unless otherwise specified.
^{b}Dofetilide tested at 5 concentrations (0.00185, 0.00556, 0.01667, 0.05000, 0.15000 μM) and run in triplicate.
^{c}Values as previously reported in J. Hill, R. M. Jones, D. Crich. ACS Med. Chem. Lett. 2023, 14, 1869-1875 and in Example 1. 33.
^{d}Mean of n = 2 independent replicates.

CYP Inhibition of CYPA2, CYP2C9, CYP2D6 and CYP3A4 in human liver microsomes

TABLE 15
CYP450 control compound details
used in the CYP inhibition assay.

| | Positive | |
|---|---|---|
| CYP Isoform | Inhibitors | Final Concentration (UM) |
| CYP1A2 | Furafylline | 0, 0.0075, 0.025, 0.075, 0.25, |
| | | 0.75, 2.5, 7.5, 25 |
| CYP2C9 | Sulfaphenazole | 0, 0.0015, 0.005, 0.015, 0.05, |
| | | 0.15, 0.5, 1.5, 5 |
| CYP2D6 | Quinidine | 0, 0.0015, 0.005, 0.015, 0.05, |
| | | 0.15, 0.5, 1.5, 5 |
| CYP3A4 | Ketoconazole | 0, 0.0015, 0.005, 0.015, 0.05, |
| | | 0.15, 0.5, 1.5, 5 |

TABLE 16
CYP450 substrate concentration details.

| | | Working | Final | |
|---|---|---|---|---|
| | | Concen- | Concen- | |
| CYP | | tration | tration | Incubation |
| Isoform | Substrate | (μM) | (μM) | Time |
| CYP1A2 | Phenacetin | 800 | 40 | 20 min |
| CYP2C9 | Diclofenac | 120 | 6 | 5 min |
| CYP2D6 | Dextromethorphan | 40 | 2 | 20 min |
| CYP3A4 | Midazolam | 20 | 1 | 5 min |

TABLE 17
Results for CYP450 inhibition assay.

| | IC₅₀ (μM)^{a} | | | |
|---|---|---|---|---|
| Compound | CYP1A2 | CYP2C9 | CYP2D6 | CYP3A4-M |
| Furafylline | 3.01 | — | — | — |
| Sulfaphenazole | — | 0.21 | — | — |
| Quinidine | — | — | 0.023 | — |
| Ketoconazole | — | — | — | 0.016 |
| 21 | >30 | 16.5 | 27.1 | 1.0 |

^{a}IC₅₀ values are presented as the mean (n = 2 independent replicates).

### Direct and Time-Dependent Inhibition of CYP3A⁴ in Human Liver Microsomes

TABLE 18
Microsome details used in direct and time-
dependent CYP3A4 inhibition study.

| Item | Supplier |
|---|---|
| Pooled Human Liver Microsomes, Mixed | BD Gentest (Cat. No. 452117) |
| Gender | |

TABLE 19
CYP3A4 inhibitor compound concentration details.

| | | Working | Final |
|---|---|---|---|
| CYP | Positive | Concentration | Concentration |
| Isoform | Control | (μM) | (μM) |
| CYP3A4 | Mifepristone | 0.02, 0.06, 0.2, | 0.1, 0.3, 1, 3, |
| | | 0.6, 2, 6 | 10, 30 |
| CYP3A4 | 21 | 6, 20, 60, 200, | 0.03, 0.1, 0.3, |
| | | 600, 2000 | 1, 3, 10 |

TABLE 20
Final CYP3A4 substrate concentrations used in assay.

| | | Working | Final | |
|---|---|---|---|---|
| CYP | | Concentration | Concentration | Incubation |
| Isoform | Substrate | (μM) | (μM) | Time |
| CYP3A4 | Midazolam | 20 | 1 | 5 min |

TABLE 21
Results for CYP3A4 time-dependent inhibition
assay in human liver microsomes.

| | | CYP2D6 | |
|---|---|---|---|
| | | | Inhibition |
| | | | percentage(%) |
| Compound | Pre-incubation | IC₅₀ (μM)^{a} | at Top conc. |
| Mifepristone | 0 min | 1.13 | 84.46 |
| | 30 min without NADPH | 1.00 | 80.51 |
| | 30 min with NADPH | 0.10 | 96.70 |
| 21 | 0 min | 1.47 | 72.41 |
| | 30 min without NADPH | 1.50 | 72.07 |
| | 30 min with NADPH | 2.64 | 76.57 |

^{a}IC₅₀ values are presented as the mean (n = 2 independent replicates).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1) Kurzrock, R.; Gutterman, J. U.; Talpaz, M. The Molecular Genetics of Philadelphia Chromosome-Positive Leukemias. N. Engl. J. Med. 1988, 319, 990-998.
2) Lugo, T. G.; Pendergast, A. M.; Muller, A. J.; Witte, O. N. Tyrosine Kinase Activity and Transformation Potency of BCR-ABL Oncogene Products. Science 1990, 247, 1079-1082.
3) Flis, S.; Chojnacki, T. Chronic Myelogenous Leukemia, a Still Unsolved Problem: Pitfalls and New Therapeutic Possibilities. Drug Des. Devel. Ther. 2019, 13, 825-843.
4) Advani, A. S.; Pendergast, A. M. Bcr-Abl Variants: Biological and Clinical Aspects. Leuk. Res. 2002, 26, 713-720.
5) Hantschel, O. Structure, Regulation, Signaling, and Targeting of AbN Kinases in Cancer. Genes Cancer 2012, 3, 436-446.
6) Druker, B. J.; Tamura, S.; Buchdunger, E.; Ohno, S.; Segal, G. M.; Fanning, S.; Zimmermann, J.; Lydon, N. B. Effects of a Selective Inhibitor of the AbN Tyrosinde Kinase on the Growth of Bcr-Abl Positive Cells. Nat. Med. 1996, 2, 561-566.
7) Hochhaus, A.; O'Brien, S. G.; Guilhot, F.; Druker, B. J.; Branford, S.; Foroni, L.; Goldman, J. M.; Muller, M. C.; Radich, J. P.; Rudoltz, M.; Mone, M.; Gathmann, I.; Hughes, T. P.; Larson, R. A. IRIS Investigators. Six-Year Follow-up of Patients Receiving Imatininb for the First-Line Treatment of Chronic Myeloid Leukemia. Leukemia 2009, 23, 1054-1061.
8) Druker, B. J.; Guilhot, F.; O'Brien, S. G.; Gathmann, I.; Kantarjian, H.; Gattermann, N.; Deininger, M. W. N.; Silver, R. T.; Goldman, J. M.; Stone, R. M.; Cervantes, F.; Hochhaus, A.; Powell, B. L.; Gabrilove, J. L.; Rousselot, P.; Reiffers, J.; Cornelissen, J. J.; Hughes, T.; Agis, H.; Fischer, T.; Verhoef, G.; Shepherd, J.; Saglio, G.; Gratwohl, A.; Nielson, J. L.; Radich, J. P.; Simonsson, B.; Taylor, K.; Baccarani, M.; So, C.; Letvak, L.; Larson, R. A. IRIS Investigators. Five-Year Follow-up of Patients Receiving Imatinib for Chronic Myeloid Leukemia. N. Enlg. J. Med. 2006, 355, 2408-2417.
9) Cohen, P.; Cross, D.; Janne, P. A. Kinase Drug Discovery 20 Years after Imatinib: Progress and Future Directions. Nat. Rev. Drug Discov. 2021, 20, 551-569.
10) Attwood, M. M.; Fabbro, D.; Sokolov, A. V.; Knapp, S.; Schlöth, H. B. Trends in Kinase Drug Discover: Targets, Indications and Inhibitor Design. Nat. Rev. Drug Discov. 2021, 20, 839-861.
11) Ayala-Aguilera, C. C.; Valero, T.; Lorente-Macias, Á.; Baillache, D. J.; Croke, S.; Unciti-Broceta, A. Small Molecule Kinase Inhibitor Drugs (1995-2021): Medical Indication, Pharmacology, and Synthesis. J. Med. Chem. 2022, 65, 1047-1131.
12) Gorre, M. E.; Mohammed, M.; Ellwood, K.; Hsu, N.; Paquette, R.; Rao, P. N.; Sawyer, C. L. Clinical Resistance to STI-571 Cancer Therapy Caused by BCR-ABL Gene Mutation or Amplification. Science 2001, 293, 876-880.
13) Von Bubnoff, N.; Schneller, F.; Peschel, C.; Duyster, J. BCR-ABL Gene Mutation in Relation to Clinical Resistance of Philadelphia-Chromosome-Positive Leukaemia to ST1571: A Prospective Study. Lancet 2002, 359, 487-491.
14) Hoschhaus, A.; Kreil, S.; Corbin, A. S.; La Rosée, P.; Muller, M. C.; Lahaye, T.; Hanfstein, C.; Schoch, C.; Cross, N. C. P.; Berger, U.; Gschaidmeier, H.; Druker, B. J.; Hehlmann, R. Molecular and Chromosomal Mechanisms of Resistance to Imatinib (ST1571) Therapy. Leukemia 2002, 16, 2190-2196.
15) Apperley, J. F. Part II: Management of Resistance to Imatinib in Chronic Myeloid Leukaemia. Lancet Oncol. 2007, 8, 1116-1128.
16) Braun, T. P.; Eide, C. A.; Druker, B. J. Response and Resistance to BCR-ABL1-Targeted Therapies. Cancer Cell 2020, 37, 530-542.
17) Lombardo, L. J.; Lee, F. Y.; Chen, P.; Norris, D.; Barrish, J. C.; Behnia, K.; Castaneda, S.; Cornelius, L. A. M.; Das, J.; Doweyko, A. M.; Fairchild, C.; Hunt, J. T.; Inigo, I.; Johnstrom, K.; Kamath, A.; Kan, D.; Klei, H.; Marathe, P.; Pang, S.; Peterson, R.; Pitt, S.; Schieven, G. L.; Schmidt, R. J.; Tokarski, J.; Wen, M.-L.; Wityak, J.; Borzilleri, R. M. Discovery of N-(2-Chloro-6-Methyl-Phenyl)-2-(6-(4-(2-Hydroxyethyl)-Piperazin-1-Yl)-2-Methylpyridimidin-4-Ylamino)Thiazole-5-Carboxamide (BMS-354825), a Dual Src/Abl Kinase Inhibitor with Potent Antitumor Activity in Preclinical Assays. J. Med. Chem. 2004, 47, 6658-6661.
18) Weisberg, E.; Manley, P. W.; Breitenstein, W.; Bruggen, J.; Cowan-Jacob, S. W.; Ray, A.; Huntly, B.; Fabbro, D.; Fendrich, G.; Hall-Meyers, E.; Kung, A. L.; Mestan, J.; Daley, G. Q.; Callahan, L.; Catley, L.; Cavazza, C.; Mohammed, A.; Neuberg, D.; Wright, R. D.; Gilliland, D. G.; Griffin, J. D. Characterization of AMN107, a Selective Inhibitor of Native and Mutant Bcr-Abl. Cancer Cell 2005, 7, 129-141.
19) Puttini, M.; Colucia, A. M. L.; Boschelli, F.; Cleris, L.; Marchesi, E.; Donella-Deana, A.; Ahmed, S.; Redaelli, S.; Piazza, R.; Magistroni, V.; Andreoni, F.; Scapozza, L.; Formelli, F.; Gambacorti-Passerini, C. In Vitro and In Vivo Activity of SKI-606, a Novel Src-Abl Inhibitor, Against Imatininb-Resistant Bcr-Abl+ Neoplastic Cells. Cancer Res. 2006, 66, 11314-11322.
20) Huang, W.-S.; Metcalf, C. A.; Sundaramoorthi, R.; Wang, Y.; Zou, D.; Thomas, R. M.; Zhu, X.; Cai, L.; Wen, D.; Liu, S.; Romero, J.; Qi, J.; Chen, I.; Banda, G.; Lentini, S. P.; Das, S.; Xu, Q.; Keats, J.; Wang, F.; Wardwell, S.; Ning, Y.; Snodgrass, J. T.; Broudy, M. I.; Russian, K.; Zhou, T.; Commodore, L.; Narasimhan, N. I.; Mohemmad, Q. K.; Luliucci, J.; Rivera, V. M.; Dalgarno, D. C.; Sawyer, T. K.; Clackson, T.; Shakespeare, W. C. Discovery of 3-[2-(Imidazo[1,2-b] Pyridazin-3-YI)Ethynyl]-4-Methyl-N-{4-[(4-Methylpiperazin-1-Yl)Methyl]-3-(Trifluoromethyl)Phenyl}benzamide (AP24534), a Potent, Orally Active Pan-Inhibitor of Breakpoint Cluster Region-Abelson (BCR-ABL) Kinase Including the T315I Gatekeeper Mutant. J. Med. Chem. 2010, 53, 4701-4719.
21) Schoepfer, J.; Jahnke, W.; Berellini, G.; Buonamici, S.; Cotesta, S.; Cowan-Jacob, S. W.; Dodd, S.; Drueckes, P.; Fabbro, D.; Gabriel, T.; Groell, J.-M.; Grotzfeld, R. M.; Hassan, A. Q.; Henry, C.; lyer, V.; Jones, D.; Lombardo, F.; Loo, A.; Manley, P. W.; Pellé, X.; Rummel, G.; Salem, B.; Warmuth, M.; Wylie, A. A.; Zoller, T.; Marzinzik, A. L.; Furet, P.; Discovery of Asciminib (ABL001), an Allosteric Inhibitor of Tyrosine Kinase Activity of BCR-ABL1. J. Med. Chem. 2018, 61, 8120-8135.
22) Lamore, S. D.; Kohnken, R. A.; Peters, M. F.; Kolaja, K. L. Cardiovascular Toxicity Induced by Kinase Inhibitors: Mechanisms and Preclinical Approaches. Chem. Res. Toxicol. 2020, 33, 125-136.
23) Doherty, K. R.; Wappel, R. L.; Talbert, D. R.; Trusk, P. B.; Moran, D. M.; Kramer, J. W.; Brown, A. M.; Shell, S. A.; Bacus, S. Multi-Parameter In Vitro Toxicity Testing of Crizotinib, Sunitinib, Erlotinib, and Nilotinib in Human Cardiomyocytes. Toxicol. Appli. Pharmacol. 2013, 272, 245-255.
24) Goldenberg, M. M. Pharmaceutical Approval Update. Pharmacy and Therapeutics 2008, 33, 54-57.
25) Rivera, V. M.; Pritchard, J. R.; Gonzalvez, F.; Baker, T.; Gozgit, J. M.; Hodgson, G. Comparitive TKI Profiling Analyses to Explore Potential Mechanisms of Ponatinib-Associated Arterial Thrombotic Events. Blood 2014, 124, 1783.
26) Latifi, Y.; Moccetti, F.; Wu, M.; Xie, A.; Packwood, W.; Qi, Y.; Ozawa, K.; Shentu, W.; Brown, E.; Shirai, T.; McCarty, O. J.; Ruggeri, Z.; Moslehi, J.; Chen, J.; Druker, B. J.; López, J. A.; Lindner, J. R. Thrombotic Microangiopathy as a Cause for Cardiovascular Toxicity from the BCR-ABL1 Tyrosine Kinase Inhibitor Ponatininb. Blood 2019, 133, 1597-1606.
27) Hiwase, D. K.; Saunders, V.; Hewett, D.; Frede, A.; Zrim, S.; Dang, P.; Eadie, L.; Bik To, L.; Melo, J.; Kumar, S.; Hughes, T. P.; White, D. L. Dasatinib Cellular Uptake and Efflux in Chronic Myeloid Leukemia Cells: Therapeutic Implications. Clin. Cancer Res. 2008, 14, 3881-3888.
28) Eadie, L. N.; Saunders, V. A.; Branford, S.; White, D. L.; Hughes, T. P. The New Allosteric Inhibitor Asciminib is Susceptible to Resistance Mediated by ABCB1 and ABCG2 Overexpression In Vitro. Oncotarget 2018, 9, 13423-13437.
29) Redaelli, S.; Perini, P.; Ceccon, M.; Piazza, R.; Rigolio, R.; Mauri, M.; Boschelli, F.; Giannoudis, A.; Gambacorti-Passerini, C. In Vitro and In Vivo Identification of ABCB1 as an Efflux Transporter of Bosutinib. J. Hematol. Oncol. 2015, 8, 81.
30) Sun, Y.-L.; Patel, A.; Kumar, P.; Chen, Z.-S. Role of ABC Transporters in Cancer Chemotherapy. Chin. J. Cancer. 2012, 31, 51-57.
31) Hill, J.; Jones, R. M.; Crich, D. Atypical N-Alkyl to N-Noralkoxy Switch in a Dual cSRC/BCR-AB11 Kinase Inhibitor Improves Drug Efflux and hERG Affinity. ACS Med. Chem. Lett. 2023, 14, 1869-1875.
32) Barlaam, B.; Fennell, M.; Germain, H.; Green, T.; Hennequin, L.; Morgentin, R.; Olivier, A.; Ple, P.; Vautier, M.; Costello, G. New Heterocyclic Analogues of 4-(2-Chloro-5-methoxyanilino)quinazolines as Potent and Selective c-Src Kinase Inhibitors. Bioorg. Med. Chem. Lett. 2005, 15, 5446-5449.
33) Still, W. C.; Kahn, M.; Mitra, A. Rapid Chromatographic Technique for Preparative Separations with Moderate Resolution. J. Org. Chem. 1978, 43, 2923-2935.
34) Hill, J.; Hettikankanamalage, A. A.; Crich, D. Diversity-Oriented Synthesis of N,N,O-Trisubstituted Hydroxylamines from Alcohols and Amines by N—O Bond Formation. J. Am. Chem. Soc. 2020, 142, 14820-14825.
35) Hill, J.; Crich, D. The N,N,O-Trisubstituted Hydroxylamine Isostere and Its Influence on Lipophilicity and Related Parameters. ACS Med. Chem. Lett. 2022, 13, 799-806.
36) Puttini, M.; Coluccia, A. M. L.; Boschelli, F.; Cleris, L.; Marchesi, E.; Donella-Deana, A.; Ahmed, S.; Redaelli, S.; Piazza, R.; Magistroni, V.; Andreoni, F.; Scapozza, L.; Formelli, F.; Cambacorti-Passerini, C. In Vitro and In Vivo Activity of SKI-606, a Novel Src-Abl Inhibitor against Imatinib-Resistant Bcr-Abl⁺ Neoplastic Cells. Cancer Res. 2006, 66, 11314-11322.
37) Nicolini, F. E.; Mauro, M. J.; Martinelli, G.; Kim, D.-W.; Soverini, S.; Muller, M. C.; Hochhaus, A.; Cortes, J.; Chuah, C.; Dufva, I. H.; Apperley, J. F.; Yagasaki, F.; Pearson, J. D.; Peter, S.; Sanz Rodriguez, C.; Preudhomme, C.; Giles, F.; Goldman, J. M.; Zhou, W. Epidemiologic Study on Survival of Chronic Myeloid Leukemia and Ph(+) Acute Lymphoblastic Leukemia Patients with BCR-ABL T315I Mutation. Blood 2009, 114, 5271-5278.
38) Yazdanian, M.; Glynn, S. L.; Wright, J. L.; Hawi, A. Correlating Partitioning and Caco-2 Cell Permeability of Structurally Diverse Small Molecular Weight Compounds. Pharm. Res. 1998, 15, 1490-1494.
39) Breeman, R. B. V.; Li, Y. Caco-2 Cell Permeability Assays to Measure Drug Absorption. Expert Opin. Drug Metab. Toxicol. 2005, 1, 175-185.
40) Ames, B. N.; McCann, J.; Yamasaki, E. Methods for Detecting Carcinogens and Mutagens with the Salmonella/Mammalian-Microsome Mutagenecity Test. Mutat. Res. 1975, 31, 347-364.
41) Corvi, R.; Albertini, S.; Hartung, T.; Hoffmann, S.; Maurici, D.; Pfuhler, S.; Benthem, J. V.; Vanparys, P. ECVAM Retrospective Validation of In Vitro Micronucleus Test (MNT). Mutagenesis 2008, 23, 271-283.
42) Bruns, B. F.; Watson, I. A. Rules for Identifying Potentially Reactive or Promiscuous Compounds. J. Med. Chem. 2012, 55, 9764-9772.
43) Rishton, G. M. Non-lead-likeness and Lead-likeness in Biochemical Screening. Drug Disc. Today 2003, 8, 86-96.
44) Yum, B.; Reynisson, J. Bond Stability of the “Undesirable” Heteroatom-heteroatom Molecular Moieties for High-Throughput Screening Libraries. Eur. J. Med. Chem. 2011, 46, 5822-5837.
45) Grimm, S. W.; Einolf, H. J.; Hall, S. D.; He, K.; Lim, H.-K.; Ling, K.-H. J.; Lu, C.; Nomeir, A. A.; Seibert, E.; Skordos, K. W.; Tonn, G. R.; Horn, R. V.; Wang, R. W.; Wong, Y. N.; Yang, T. J.; Obach, R. S. The Conduct of In Vitro Studies to Address Time-Dependent Inhibition of Drug-Metabolizing Enzymes: A Perspective of the Pharmaceutical Research and Manufacturers of America. Drug Metab. Dispos. 2009, 37, 1355-1370.
46) Wong, S.; Witte, O. N. The BCR-ABL Story: Bench to Bedside and Back. Annu. Rev. Immunol. 2004, 22, 247-306.
47) Quintás-Cardama, A.; Cortes, J. Molecular Biology of bcr-ab/1-Positive Chronic Myeloid Leukemia. Blood 2009, 113, 1619-1630.
48) Fors, B. P.; Buchwald, S. L. A Multiligand Based Pd Catalyst for C—N Cross-Coupling Reactions. J. Am. Chem. Soc. 2010, 132, 15914-15917.
49) Kanofsky, J. R.; Sima, P. D. Preferential cytotoxicity for multidrug-resistant K562 cells using the combination of a photosensitizer and a cyanine dye. J. Photochem. Photobiol. B: Biol. 2000, 54, 136-144.
50) Boschelli, D. H.; Wang, Y. D.; Johnson, S.; Wu, B.; Ye, F.; Sosa, A. C. B.; Golas, J. M.; Boschelli, F. 7-Alkoxy-4-phenylamino-4-quinolinecar-bonitriles as Dual Inhibitors of Src and AbN Kinases. J. Med. Chem. 2004, 47, 1599-1601.
51) Boschelli, D. H.; Wu, B.; Sosa, A. C. B.; Durutlic, H.; Chen, J. J.; Wang, Y.; Golas, J. M.; Lucas, J.; Boschelli, F. Synthesis and Src Kinase Inhibitory Activity of 2-Phenyl- and 2-Thienyl-7-phenylaminothieno[3,2-b]pyridine-6-carbonitriles. J. Med. Chem. 2005, 48, 3891-3902.
52) Mahon, F.-X.; Belloc, F.; Lagarde, V.; Chollet, C.; Moreau-Gaudry, F.; Reiffers, J.; Goldman, J. M.; Melo, J. V. MDR1 Gene Overexpression Confers Resistance to Imatinib Mesylate in Leukemia Cell Line Models. Blood 2003, 101, 2368-2373.
53) Eadie, L. N.; Hughes, T. P.; White, D. L. ABCB1 Overexpression is a Key Initiator of Resistance to Tyrosine Kinase Inhibitors in CML Cell Lines. PLOS One 2016, 11, e0161470.

1. A compound having structure I or the pharmaceutically acceptable salt thereof 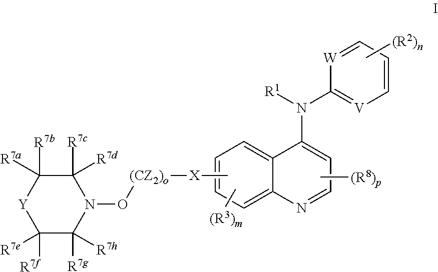
wherein
R¹ is hydrogen, a substituted or unsubstituted linear or branched alkyl group, a substituted or unsubstituted cycloalkyl group or heterocycloalkyl group, or a substituted or unsubstituted aryl group;
n is an integer from 1 to 5, where each R² is independently hydrogen, a substituted or unsubstituted linear or branched alkyl group, a substituted or unsubstituted cycloalkyl group or heterocycloalkyl group, a substituted or unsubstituted aryl group, a halide, or an alkoxy group;
m is an integer from 1 to 3, where each R³ is independently hydrogen, a substituted or unsubstituted linear or branched alkyl group, a substituted or unsubstituted cycloalkyl group or heterocycloalkyl group, a substituted or unsubstituted aryl group, a halide, or an alkoxy group;
o is an integer from 1 to 10;
p is 1 or 2;
V is N or CH;
W is N or CH;
X is O or NR⁴, wherein R⁴ is hydrogen, a substituted or unsubstituted linear or branched alkyl group, a substituted or unsubstituted cycloalkyl group or heterocycloalkyl group, or a substituted or unsubstituted aryl group;
Y is O, NR⁵, or CR^{6a}R^{6b}, wherein R⁵ is hydrogen, deuterium, a substituted or unsubstituted linear or branched alkyl group, a substituted or unsubstituted cycloalkyl group or heterocycloalkyl group, or a substituted or unsubstituted aryl group, and
R^{6a} and R^{6b} are independently hydrogen, deuterium, a substituted or unsubstituted linear or branched alkyl group, a substituted or unsubstituted cycloalkyl group or heterocycloalkyl group, a substituted or unsubstituted aryl group, or a substituted or unsubstituted amino group;
R^{7a}, R^{7b}, R^{7c}, R^{7d}, R^{7e}, R^{7f}, R^{7g}, and R^{7h} are independently hydrogen, deuterium, or a substituted or unsubstituted alkyl group;
R³ is cyano; and
each Z is independently hydrogen or deuterium.

2. (canceled)

3. (canceled)

4. The compound of claim 1, wherein X is O, Y is O or NR⁵, where R⁵ is a C1 to C5 alkyl group, or Y is CR^{6a}R^{6b}, where R^{6a} is hydrogen and R^{6b} is a substituted or unsubstituted amino group.

5. (canceled)

6. The compound of claim 1, wherein R¹ is hydrogen, R³ is an alkoxy group, m is 1, n is 2 or 3, o is an integer from 1 to 5.

7-13. (canceled)

14. The compound of claim 1, wherein p is 1.

15. The compound of claim 1, wherein (a) W is CH and V is CH or N (b) Wis N and V is N.

16. (canceled)

17. (canceled)

18. The compound of claim 1, wherein the compound has the structure II or the pharmaceutically acceptable salt thereof 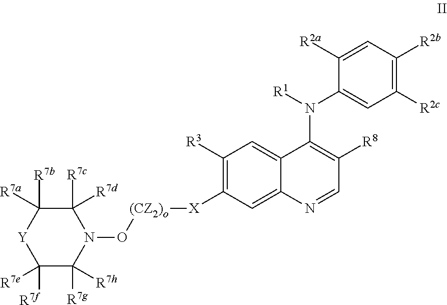
wherein
R¹ is hydrogen, a substituted or unsubstituted linear or branched alkyl group, a substituted or unsubstituted cycloalkyl group or heterocycloalkyl group, or a substituted or unsubstituted aryl group;
R^{2a} and R^{2b} are a halide;
R^{2c} is an alkoxy group;
R³ is an alkoxy group;
o is an integer from 1 to 5;
X is O or NR⁴, wherein R⁴ is hydrogen, a substituted or unsubstituted linear or branched alkyl group, a substituted or unsubstituted cycloalkyl group or heterocycloalkyl group, or a substituted or unsubstituted aryl group;
Y is O, NR⁵, or CR^{6a}R^{6b}, wherein R⁵ is hydrogen, deuterium, a substituted or unsubstituted linear or branched alkyl group, a substituted or unsubstituted cycloalkyl group or heterocycloalkyl group, or a substituted or unsubstituted aryl group, and
R^{6a} and R^{6b} are independently hydrogen, deuterium, a substituted or unsubstituted linear or branched alkyl group, a substituted or unsubstituted cycloalkyl group or heterocycloalkyl group, a substituted or unsubstituted aryl group, or a substituted or unsubstituted amino group;
R^{7a}, R^{7b}, R^{7c}, R^{7d}, R^{7e}, R^{7f}, R^{7g}, and R^{7h} are independently hydrogen, deuterium, or a substituted or unsubstituted alkyl group;
R³ is cyano; and
each Z is independently hydrogen or deuterium.

19. (canceled)

20. (canceled)

21. The compound of claim 18, wherein X is O, Y is O or NR⁵, where R⁵ is a C1 to C5 alkyl group, Y is CR^{6a}R^{6b}, where R^{6a} is hydrogen and R^{6b} is a substituted or unsubstituted amino group.

22. (canceled)

23. (canceled)

24. The compound of claim 18, wherein R¹ is hydrogen, R³ is a C1 to C10 substituted or unsubstituted linear or branched alkoxy group, o is an integer from 1 to 5.

25. (canceled)

26. (canceled)

27. The compound of claim 18, wherein R^{2a} and R^{2b} are chloride, or R^{2a} is chloride and R^{2b} is fluoride, or R^{2a} is fluoride and R^{2b} is chloride.

28. (canceled)

29. (canceled)

30. The compound of claim 18, wherein R^{2c} is a C1 to C10 substituted or unsubstituted linear or branched alkoxy group.

31. (canceled)

32. The compound of claim 1, wherein the compound has the structure III, IV, V, or VI or the pharmaceutically acceptable salt thereof 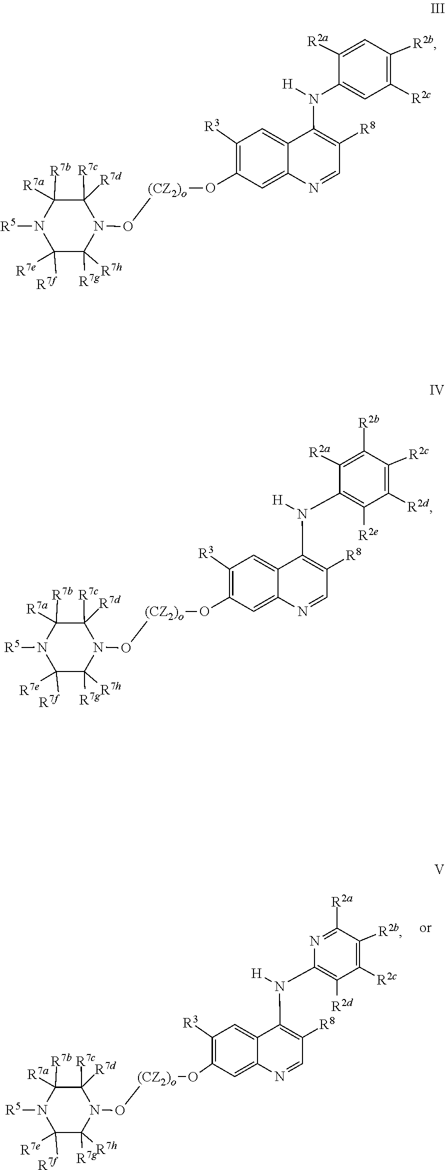 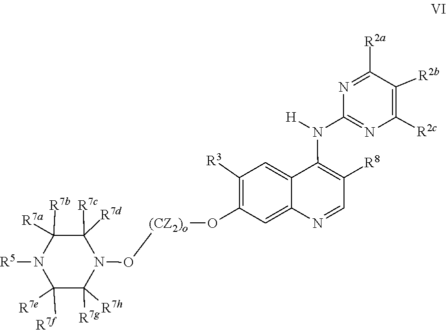
wherein
R^{2a} and R^{2b} are a halide;
R^{2c} is an alkoxy group;
R³ is an alkoxy group;
o is an integer from 1 to 5;
R⁵ is hydrogen, deuterium, a substituted or unsubstituted linear or branched alkyl group, a substituted or unsubstituted cycloalkyl group or heterocycloalkyl group, or a substituted or unsubstituted aryl group, and
R^{7a}, R^{7b}, R^{7c}, R^{7d}, R^{7e}, R^{7f}, R^{7g}, and R^{7h} are independently hydrogen, deuterium, or a substituted or unsubstituted alkyl group;
R³ is cyano; and
each Z is independently hydrogen or deuterium.

33. The compound of claim 32, wherein R⁵ is a C1 to C5 alkyl group.

34. (canceled)

35. The compound of claim 32, wherein R³ is a C1 to C10 substituted or unsubstituted linear or branched alkoxy group.

36. (canceled)

37. The compound of claim 32, wherein o is an integer from 1 to 5.

38. (canceled)

39. The compound of claim 32, wherein R^{2a} is chloride and R^{2b} is fluoride, or R^{2a} is fluoride and R^{2b} is chloride, or R^{2a} and R^{2b} are chloride.

40. (canceled)

41. (canceled)

42. The compound of claim 32, wherein R^{2c} is a C1 to C10 substituted or unsubstituted linear or branched alkoxy group.

43. (canceled)

44. The compound of claim 32, wherein R^{7a}, R^{7b}, R^{7c}, R^{7d}, R^{7e}, R^{7f}, R^{7g}, and R^{7h} are each hydrogen.

44-77. (canceled)

78. The compound of claim 1, wherein the compound is

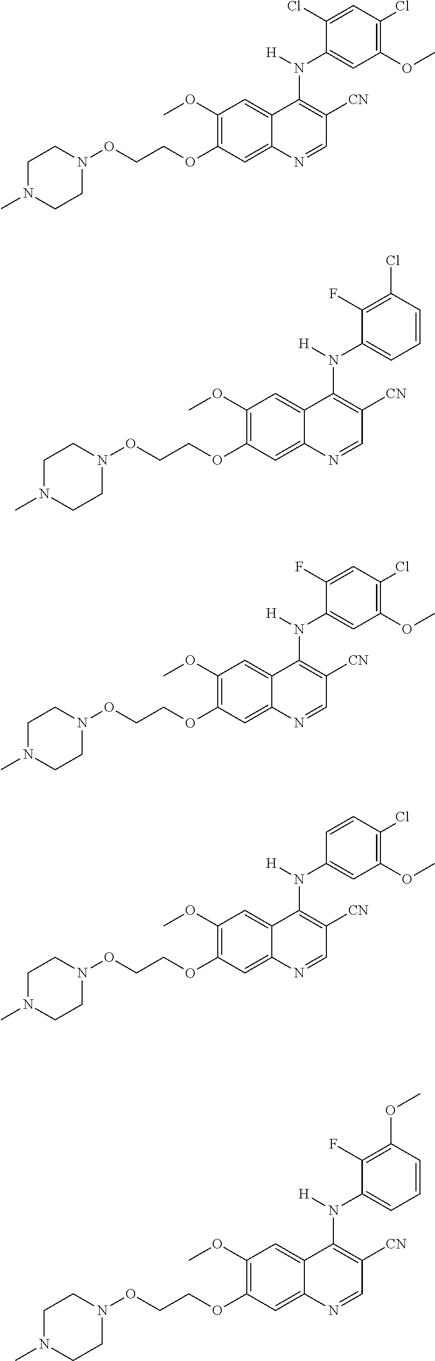

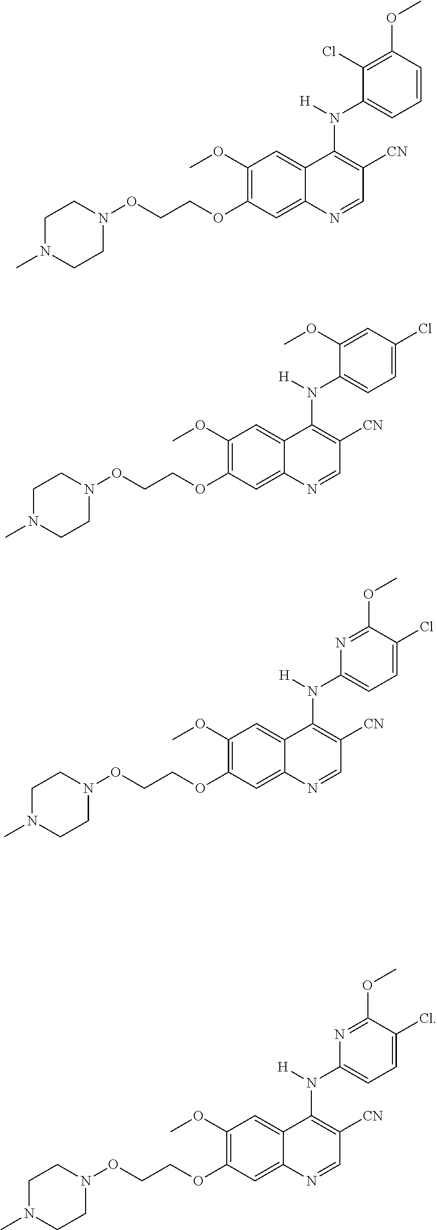

79. A pharmaceutical composition comprising the compound of claim 1 and a pharmaceutically-acceptable carrier.

80. A method for treating a subject having chronic myeloid leukemia, acute lymphoblastic leukemia, glioblastoma, non-small cell lung cancer, prostate cancer, or colorectal cancer, the method comprising administering to the subject an effective amount of the compound of claim 1.

81-85. (canceled)
